(19) United States
(12) Patent Application Publication
(10) Pub. No.: US 2014/0113934 A1
(43) Pub. Date: Apr. 24, 2014

(54) HETEROCYCLIC COMPOUNDS FOR TREATING HELMINTH INFECTIONS

(71) Applicants: George Philip Lahm, Wilmington, DE (US); Benjamin Kenneth Smith, Newark, DE (US)

(72) Inventors: George Philip Lahm, Wilmington, DE (US); Benjamin Kenneth Smith, Newark, DE (US)

(73) Assignee: E I DU PONT DE NEMOURS AND COMPANY, Wilmington, DE (US)

(21) Appl. No.: 14/124,730

(22) PCT Filed: Jun. 19, 2012
(86) PCT No.: PCT/US2012/043195

§ 371 Date: Dec. 9, 2013

Related U.S. Application Data
(60) Provisional application No. 61/498,888, filed on Jun. 20, 2011.

(51) Int. Cl. C07D 215/14; C07D 401/12; A61K 31/4709; C07D 409/12; A61K 31/47

(52) U.S. Cl. CPC C07D 215/14; C07D 409/12; A61K 31/47; A61K 31/4709; C07D 401/12
USPC 514/314; 546/172; 514/311

(57) ABSTRACT

Disclosed are compounds of Formula 1, N-oxides, and salts thereof,

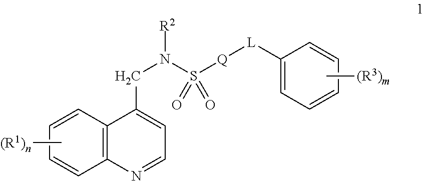

wherein
L is (CR^{13a}R^{13b})ₜ, CR¹⁴═CR¹⁴, C≡C, CR^{15a}R^{15b}X, XCR^{15a}R^{15b}, CO, O, S(O)ₚ, NR⁵, CONR⁵, NR⁵CO, NR⁵SO₂ or SO₂NR⁵;
X is O, S, SO, SO₂, or NR⁵;
and Q, R¹, R², R³, R⁵, R^{13a}, R^{13b}, R¹⁴, R^{15a}, R^{15b} t, p, n and m are as defined in the disclosure.
   Also disclosed are compositions containing the compounds of Formula 1 and methods for treating helminth infections comprising administration to an animal a parasiticidally effective amount of a compound or a composition of the invention.

### FIELD OF THE INVENTION

This invention relates to certain quinoline compounds, their N-oxides, salts and their compositions suitable for animal health uses and methods of their use for treating helminth infections in animals.

### BACKGROUND OF THE INVENTION

The control of animal parasites in animal health is essential, especially in the areas of food production and companion animals. Existing methods of treatment and parasite control are being compromised due to growing resistance to many current commercial parasiticides. The need continues for new compounds that are more effective, less costly, less toxic or have different sites of action to control animal parasites.

World Patent Application Publication WO 2006/097488 discloses pyridine compounds of Formula i for combating arthropodal pests.

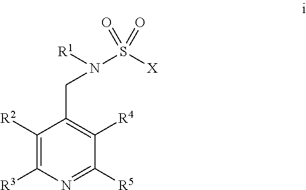

The quinoline compounds of the present invention are not disclosed in this publication.

### SUMMARY OF THE INVENTION

This invention is directed to compounds of Formula 1 (including all stereoisomers), N-oxides, and salts thereof, and compositions containing them and their use for treating helminth infections in animals:

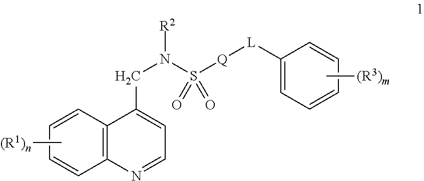

wherein
Q is phenyl or naphthalenyl each optionally substituted with up to 5 substituents independently selected from R^{4a}; or
Q is a 5- to 6-membered heteroaromatic ring or an 8- to 11-membered heteroaromatic bicyclic ring system, each ring or ring system containing ring members selected from carbon atoms and up to 4 heteroatoms independently selected from up to 2 O, up to 2 S and up to 4 N atoms, and optionally substituted with up to 5 substituents independently selected from R^{4a} on carbon atom ring members and R^{4b} on nitrogen atom ring members;
L is (CR^{13a}R^{13b})ₜ, CR¹⁴═CR¹⁴, C≡C, CR^{15a}R^{15b}X, XCR^{15a}R^{15b}, CO, O, S(O)ₚ, NR⁵, CONR⁵, NR⁵CO, NR⁵SO₂ or SO₂NR⁵;
X is O, S, SO, SO₂, or NR⁵;
each R¹ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
R² is hydrogen, cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₁-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
each R³ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
each R^{4a} is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
R^{4b} is cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
R⁵ is hydrogen, cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
each R⁶ is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, C₁-C₆ alkoxy and S(O)ₚR¹²;
each R^{7a} is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, C₁-C₆ alkoxy and S(O)ₚR¹²;
each R^{7b} is independently hydrogen; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹;
R⁸, R⁹, R¹⁰ and R¹² are each independently hydrogen; or C₁-C₆ alkyl, C₂-C₆ alkenyl, C₂-C₆ alkynyl, phenyl, benzyl, C₃-C₇ cycloalkyl, C₄-C₈ cycloalkylalkyl or C₅-C₇ cycloalkenyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₂-C₆ alkoxycarbonyl, C₂-C₆ alkylaminocarbonyl, C₂-C₈ dialkylaminocarbonyl, C₁-C₄ alkylsulfenyl, C₁-C₄ alkylsulfinyl, C₁-C₄ alkylsulfonyl, C₁-C₄ haloalkylsulfinyl, C₁-C₄ haloalkylsulfinyl and C₁-C₄ haloalkylsulfonyl;
each R¹¹ is independently hydrogen; or C₁-C₆ alkyl, C₁-C₆ alkenyl, C₂-C₆ alkynyl or benzyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl and C₁-C₄ haloalkylsulfonyl;
each R^{13a} is independently hydrogen, halogen, cyano, hydroxyl or amino; or C₂-C₆ alkoxycarbonyl, C₂-C₆ alkylaminocarbonyl or C₂-C₈ dialkylaminocarbonyl; or C₁-C₆ alkyl, C₁-C₆ alkenyl, C₂-C₆ alkynyl, C₃-C₇ cycloalkyl, C₄-C₈ cycloalkylalkyl, phenyl or benzyl each optionally substituted with substituents independently selected from the group consisting of halogen, cyano, nitro, C₁-C₄ alkyl, C₁-C₄ alkoxy, C₁-C₄ haloalkyl, C₁-C₄ haloalkoxy, C₂-C₆ alkoxycarbonyl, C₁-C₆ alkylaminocarbonyl and C₂-C₈ dialkylaminocarbonyl;
each R^{13b} is independently hydrogen, halogen, or C₁-C₆ alkyl;
each R¹⁴ is independently hydrogen, halogen, cyano, C₁-C₆ alkyl or C₁-C₆ haloalkyl;
R^{15a} is hydrogen, cyano, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₇ cycloalkyl, C₄-C₈ cycloalkylalkyl, C₅-C₇ cycloalkenyl, phenyl, benzyl, C₂-C₆ alkoxycarbonyl, C₂-C₆ alkylaminocarbonyl or C₂-C₈ dialkylaminocarbonyl;
R^{15b} is hydrogen or C₁-C₆ alkyl;
n is 0, 1, 2, 3, 4 or 5;
m is 0, 1, 2, 3, 4 or 5;
p is 0, 1 or 2; and
t is 1, 2 or 3;
provided that when
L is an oxygen atom, then one R³ group is other than hydrogen, halogen, C₁-C₄ alkyl, C₁-C₄ alkoxy, C₁-haloalkyl or C₁-haloalkoxy.

This invention is also directed to such compounds of Formula 1 (including all stereoisomers), N-oxides, and salts thereof, and compositions containing them and their use for treating, controlling, preventing or protecting animals against infection by helminths.

This invention also provides a composition comprising a parasiticidally effective amount of compounds of Formula 1, an N-oxide, or a salt thereof, and at least one pharmaceutically or veterinarily acceptable carrier or diluent. In one embodiment, this invention also provides a composition comprising a parasiticidally effective amount of a compound of Formula 1, an N-oxide, or a salt thereof, and at least one pharmaceutically or veterinarily acceptable carrier or diluent, said composition further comprising at least one additional biologically active compound or agent.

This invention provides a method for treating, controlling, preventing or protecting animals against infection by helminths which comprises orally, topically, parenterally or subcutaneously administering to the animals a parasiticdally effective amount of a compound of Formula 1, an N-oxide, or a pharmaceutically or veterinarily acceptable salt or a composition comprising it.

### DETAILS OF THE INVENTION

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “endoparasite” is a parasite that lives inside an animal and “ectoparasite” is a parasite that lives on the surface of an animal.

As referred to in this disclosure, the term “helminths” includes heartworms, roundworms (Nematoda), flukes (Tematoda), Acanthocephala and tapeworms (Cestoda).

In the context of this disclosure “controlling helminths” means inhibition or disruption of the life cycle of a helminth (including maturation, mortality, feeding reduction, and/or mating disruption), and related expressions are defined analogously. As referred to in the present disclosure the term “anthelmintic” refers to substances (drugs) that are useful in controlling helminths for example by facilitating the expulsion of parasitic worms (helminths) from the body of an animal by either stunning or killing them.

“Controlling an infestation” means both reducing the severity of the infestation as well as completely eliminating the infestation.

Animal health applications include treating an animal in need of such treatment with a compound of the invention to control a present infestion with a helminthic parasitic pest by administering a parasiticidally effective amount of a compound of the invention, typically in the form of a composition formulated for veterinary or pharmaceutical use, to the animal. Additionally the invention contemplates the prophalactic treatment of an animal in need of such treatment with a compound of the invention such that infection with a helminthic parasitic pest is prevented lessened in severity (in comparison to a similarly situated animal in an untreated state) by administering a parasiticidally effective amount of a compound of the invention, typically in the form of a composition formulated for veterinary or pharmaceutical use, to the animal to be protected. An animal can be either human (pharmaceutical use) or non-human (veterinary use).

The term “preventing infestation” means preventing infestation entirely and also reducing the severity of any infestation which may otherwise occur in the absence of treatment.

A “parasiticidally effective amount” is the amount of active ingredient needed to achieve an observable effect diminishing the occurrence or activity of the helminthic parasite. Parasiticidal effects typically relate to diminishing the occurrence or activity of the target helminth parasitic pest. Such effects on the pest include necrosis, death, retarded growth, diminished mobility or lessened ability to remain in the host animal, reduced feeding and inhibition of reproduction. These effects on helminth parasite pests provide control (including prevention, reduction or elimination) of parasitic infection of the animal. One skilled in the art will appreciate that the parasiticidally effective dose can vary for the various compounds and compositions of the present invention, the desired parasiticidal effect and duration, the target pest species, the animal to be protected, the mode of application and the like, and the amount needed to achieve a particular result can be determined through simple experimentation.

“Treating” or “Treatment” as it applies to infestation refers to both the prevention and control of infestation.

“Parenteral” as a mode of administration means taken into the body or administered in a manner other than through the digestive tract, for example by injection.

“Enteral” as a mode of administration means take into the body or administered through the digestive tract for example oral administration.

“Topical” as a mode of administration means application to the skin. It is understood that topical administration may have systemic effects dependent on the compound to be administered and the formulation in which it is contained.

In the above recitations, the term “alkyl”, used either alone or in compound words such as “alkylthio” or “haloalkyl” includes straight-chain or branched alkyl such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” also includes moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃), and the different butylene isomers. “Alkenylene” denotes a straight-chain or branched alkenediyl containing one olefinic bond. Examples of “alkenylene” include CH═CH, CH₂CH═CH, CH═C(CH₃) and the different butenylene isomers. “Alkynylene” denotes a straight-chain or branched alkynediyl containing one triple bond. Examples of “alkynylene” include C≡C, CH₂C≡C, C≡CCH₂, and the different butynylene isomers.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkenyl” includes groups such as cyclopentenyl and cyclohexenyl as well as groups with more than one double bond such as 1,3- and 1,4-cyclohexadienyl. The term “cycloalkoxy” denotes cycloalkyl attached to and linked through an oxygen atom such as cyclopentyloxy and cyclohexyloxy. “Alkylcycloalkylalkyl” denotes an alkyl group substituted with alkylcycloalkyl. Examples of “alkylcycloalkylalkyl” include 1-, 2-, 3- or 4-methyl or -ethyl cyclohexylmethyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include CF₃, CH₂Cl, CH₂CF₃ and CCl₂CF₃. The terms “haloalkenyl”, “haloalkynyl” “haloalkoxy”, “haloalkylthio”, “haloalkylamino”, “haloalkylsulfinyl”, “haloalkylsulfonyl”, “halocycloalkyl”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylamino” include CF₃(CH₃)CHNH, (CF₃)₂CHNH and CH₂ClCH₂NH. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include 2-chlorocyclopropyl, 2-fluorocyclobutyl, 3-bromocyclopentyl and 4-chlorocyclohexyl. The term “halodialkyl”, either alone or in compound words such as “halodialkylamino”, means at least one of the two alkyl groups is substituted with at least one halogen atom, and independently each halogenated alkyl group may be partially or fully substituted with halogen atoms which may be the same or different. Examples of “halodialkylamino” include (BrCH₂CH₂)₂N and BrCH₂CH₂(ClCH₂CH₂)N.

“Alkoxy” includes, for example, methoxy, ethoxy, n-propoxy, isopropoxy and the different butoxy, pentoxy and hexyloxy isomers. “Alkoxyalkyl” denotes alkoxy substitution on alkyl. Examples of “alkoxyalkyl” include CH₂OCH₃, CH₂CH₂OCH₃, CH₂OCH₂CH₃, CH₂OCH₂CH₂CH₂CH₃ and CH₂CH₂OCH₂CH₃. “Alkenyloxy” includes straight-chain or branched alkenyl attached to and linked through an oxygen atom. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC≡CCH₂O, CH₃C≡CCH₂O and CH₃C≡CCH₂CH₂O.

The term “alkylsulfenyl” or “alkylthio” includes straight-chain or branched alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O) and the different butylsulfinyl, pentylsulfinyl and hexylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl, pentylsulfonyl and hexylsulfonyl isomers. The chemical abbreviations S(O) and S(═O) as used herein represent a sulfinyl moiety. The chemical abbreviations SO₂, S(O)₂ and S(═O)₂ as used herein represent a sulfonyl moiety.

“Alkylamino” denotes an NH radical substituted with straight-chain or branched alkyl. Examples of “alkylamino” include NHCH₂CH₃, NHCH₂CH₂CH₃, and NHCH₂CH(CH₃)₂. “Dialkylamino” denotes an N radical substituted independently with two straight-chain or branched alkyl groups. Examples of “dialkylamino” include N(CH₃)₂, N(CH₃CH₂CH₂)₂ and N(CH₃)CH₂CH₃. “Halodialkylamino” denotes one straight-chain or branched alkyl moiety and one straight-chain or branched haloalkyl moiety bonded to an N radical, or two independent straight-chain or branched haloalkyl moieties bonded to an N radical, wherein “haloalkyl” is as defined above. Examples of “halodialkylamino” include N(CH₂CH₃)(CH₂CH₂Cl) and N(CF₂CF₃)₂.

“Alkylcarbonyl” denotes a straight-chain or branched alkyl moiety bonded to a C(O) moiety. The chemical abbreviations C(O) and C(═O) as used herein represent a carbonyl moiety. Examples of “alkylcarbonyl” include C(O)CH₃, C(O)CH₂CH₂CH₃ and C(O)CH(CH₃)₂. Examples of “haloalkylcarbonyl” include C(O)CF₃, C(O)CCl₃, C(O)CH₂CF₃ and C(O)CF₂CF₃.

“Alkoxycarbonyl” denotes a straight-chain or branched alkyl moiety bonded to a CO₂ moiety. The chemical abbreviations CO₂, C(O)O and C(═O)O as used herein represent an oxycarbonyl moiety. Examples of “alkoxycarbonyl” include C(O)OCH₃, C(O)OCH₂CH₃, C(O)OCH₂CH₂CH₃ and C(O)OCH(CH₃)₂.

“Alkylaminocarbonyl” denotes a straight-chain or branched alkyl moiety bonded to a C(O)NH moiety. The chemical abbreviations C(O)NH, and C(O)N as used herein represent an amide moiety (i.e. an aminocarbonyl group). Examples of “alkylaminocarbonyl” include C(O)NHCH₃, C(O)NHCH₂CH₂CH₃ and C(O)NHCH(CH₃)₂. “Dialkylaminocarbonyl” denotes two independent straight-chain or branched alkyl moieties bonded to a C(O)N moiety. Examples of “dialkylaminocarbonyl” include C(O)N(CH₃)₂ and C(O)N(CH₃)(CH₂CH₃).

“Trialkylsilyl” includes 3 branched and/or straight-chain alkyl radicals attached to and linked through a silicon atom, such as trimethylsilyl, triethylsilyl and tert-butyldimethylsilyl.

“CHO” means formyl, “OCN” means —O—C≡N, and “SCN” means —S—C≡N.

The total number of carbon atoms in a substituent group is indicated by the “Cᵢ-Cⱼ” prefix where i and j are numbers from 1 to 14. For example, C₁-C₄ alkyl designates methyl through butyl; C₂ alkoxyalkyl designates CH₂OCH₃; C₃ alkoxyalkyl designates, for example, CH₃CH(OCH₃), CH₂CH₂OCH₃ or CH₂OCH₂CH₃; and C₄ alkoxyalkyl designates the various isomers of an alkyl group substituted with an alkoxy group containing a total of four carbon atoms, examples including CH₂OCH₂CH₂CH₃ and CH₂CH₂OCH₂CH₃.

When a group contains a substituent which can be hydrogen, for example R², then when this substituent is taken as hydrogen, it is recognized that this is equivalent to said group being unsubstituted. When a variable group is shown to be optionally attached to a position, for example (R¹)ₙ in Formula 1 wherein n may be 0, then hydrogen can be at the position even if not recited in the variable group definition. When one or more positions on a group are said to be “not substituted” or “unsubstituted”, then hydrogen atoms are attached to take up any free valency.

The attachment point between (R¹)ₙ and the quinoline bicyclic ring system is illustrated as floating. This means that (R¹)ₙ can be attached to any available carbon atom ring member of the quinoline bicyclic ring system.

Unless otherwise indicated, a “ring” or “ring system” as a component of Formula 1 is carbocyclic or heterocyclic. The term “ring system” denotes two or more connected rings. The term “bicyclic ring system” denotes a ring system consisting of two rings sharing two or more common atoms.

The term “ring member” refers to an atom (e.g., C, O, N or S) forming the backbone of a ring or ring system. The term “aromatic” indicates that each of the ring atoms is essentially in the same plane and has a p-orbital perpendicular to the ring plane, and that (4n+2) π electrons, where n is a positive integer, are associated with the ring or ring system to comply with Hückel's rule.

“Partially saturated” and “partially unsaturated” with reference to a ring or ring system means that the ring or ring system contains at least one double bond but the ring or ring system is not aromatic. A ring system is aromatic if at least one component ring is aromatic.

The term “carbocyclic ring” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic ring” refers to a ring having a backbone consisting of carbon atoms linked to one another by single bonds; unless otherwise specified, the remaining carbon valences are occupied by hydrogen atoms.

The terms “heterocyclic ring” or “heterocycle” denotes a ring wherein at least one of the atoms forming the ring backbone is other than carbon. Unless otherwise indicated, a heterocyclic ring can be a saturated, partially unsaturated, or fully unsaturated ring. “Saturated heterocyclic ring” refers to a heterocyclic ring containing only single bonds between ring members. “Partially saturated heterocyclic ring” refers a heterocyclic ring containing at least one double bond but which is not aromatic. The term “heteroaromatic ring” denotes a fully unsaturated aromatic ring in which at least one atom forming the ring backbone is not carbon. Typically a heteroaromatic ring contains no more than 4 nitrogens, no more than 1 oxygen and no more than 1 sulfur. Unless otherwise indicated, heteroaromatic rings can be attached through any available carbon or nitrogen by replacement of a hydrogen on said carbon or nitrogen. The term “heteroaromatic bicyclic ring system” denotes a ring system consisting of two fused rings, in which at least one of the two rings is a heteroaromatic ring as defined above.

When a radical (e.g., a 5- to 6-membered heteroaromatic ring in the definition of Q) is optionally substituted with listed substituents with the number of substituents stated (e.g., “up to 5”), then the radical may be unsubstituted or substituted with a number of substituents ranging up to the high number stated (e.g., “5”), and the attached substituents are independently selected from the substituents listed.

When a substituent (e.g., when R¹ is cycloalkyl) is a ring or ring system, it can be attached to the remainder of Formula 1 through any available ring member, unless otherwise described.

As noted above, Q is, inter alia, a 5- to 6-membered heteroaromatic ring or an 8- to 11-membered heteroaromatic bicyclic ring system, containing ring members selected from carbon atoms and up to 4 heteroatoms independently selected from up to 2 O, up to 2 S, and up to 4 N atoms, and optionally substituted with up to 5 substituents independently selected from R^{4a} on carbon atom ring members and R^{4b} on nitrogen atom ring members. In this definition the ring members selected from up to 2 O, up to 2 S and up to 4 N are optional, because the number of heteroatom ring members may be zero. When no heteroatom ring members are present, the ring or ring system is carbocyclic. If at least one heteroatom ring member is present, the ring or ring system is heterocyclic. The nitrogen atom ring members may be oxidized as N-oxides, because compounds relating to Formula 1 also include N-oxide derivatives. As the R^{4a} and R^{4b} substituents are optional, 0 to 5 substituents may be present, limited only by the number of available points of attachment.

The term “unsubstituted” in connection with a group such as a ring or ring system means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 4.

The number of optional substituents may be restricted by an expressed limitation. For example, the phrase “optionally substituted with up to 5 substituents independently selected from R^{4a}” means that 0, 1, 2, 3, 4 or 5 substituents can be present (if the number of potential connection points allows). When a range specified for the number of substituents exceeds the number of positions available for substituents on a ring, the actual higher end of the range is recognized to be the number of available positions.

When the number of optional substituents is not restricted by an expressed limitation (e.g., the phrases “optionally substituted” or “unsubstituted or substituted with at least one substituent independently selected from”), it is understood to mean that the number of optional substituents can range from 0 up to the number of positions available. One skilled in the art will appreciate that while some substituents such as halogen can be present at every available position (for example, the C₂F₅ substituent is a C₂ alkyl group substituted with the maximum number of 5 fluorine atoms), practical factors such as cost and synthetic accessibility can limit the number of occurrences of other substituents. These limitations are part of the general synthetic knowledge known to those skilled in the art. Of note are embodiments wherein in the absence of expressed limitation of number of optional substituents, the number of optional substituents is up to 3 (i.e. 0, 1, 2 or 3) if accommodated by the number of available positions.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

Compounds selected from Formula 1 (including all stereoisomers, N-oxides, and salts thereof) typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of animal parasites (i.e. are suitable for animal health use). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides, and salts thereof.

Embodiments of the present invention as described in the Summary of the Invention include those described below. In the following Embodiments Formula 1 includes stereoisomers, N-oxides, and salts thereof, and reference to “a compound of Formula 1” includes the definitions of substituents specified in the Summary of the Invention unless further defined in the Embodiments.

In the Embodiments that follow, recitation of the word “independently” before more than one variable being defined means that the definition can be applied to each variable independently of the other variables.

### Embodiment 1

A compound of Formula 1 wherein Q is a ring selected from the group consisting of Q-1 through Q-42 in Exhibit 1

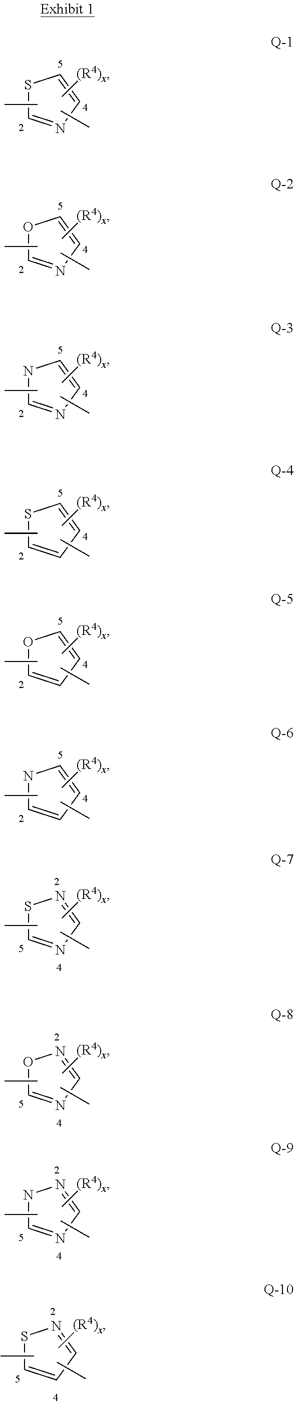

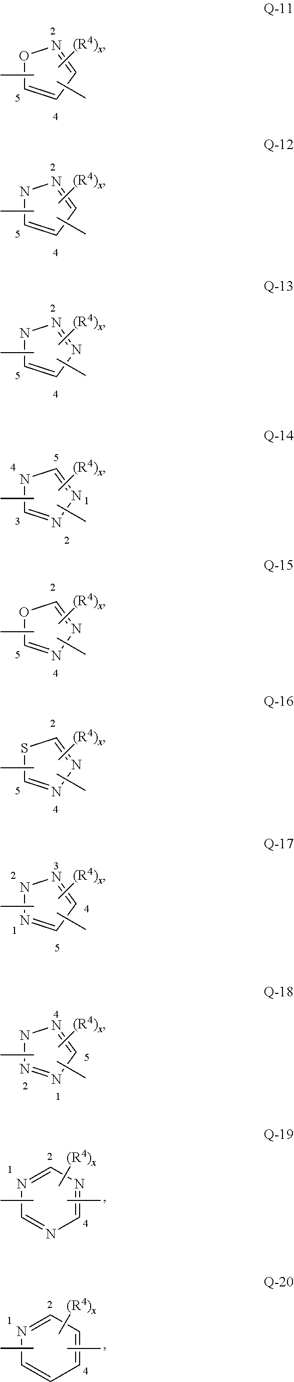

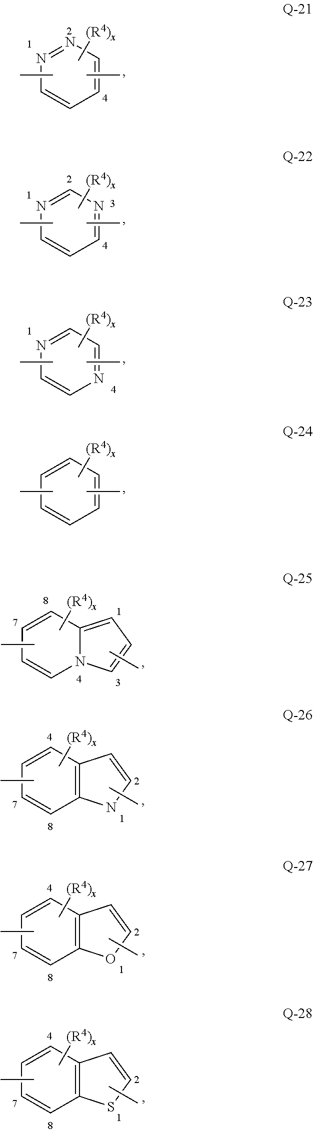

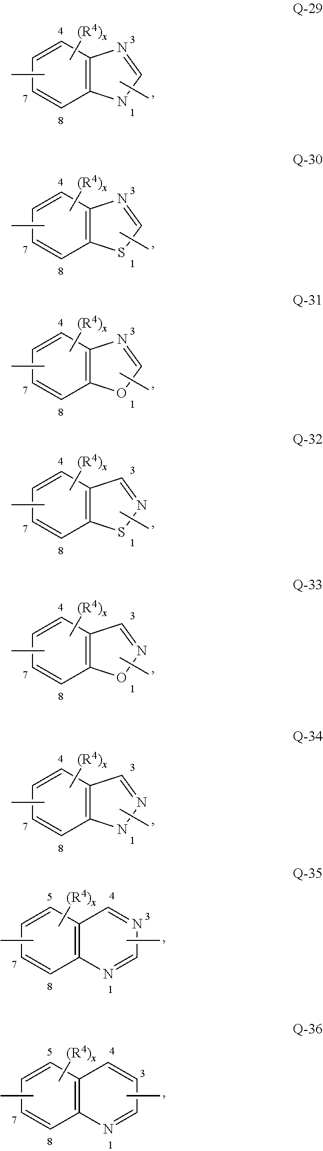

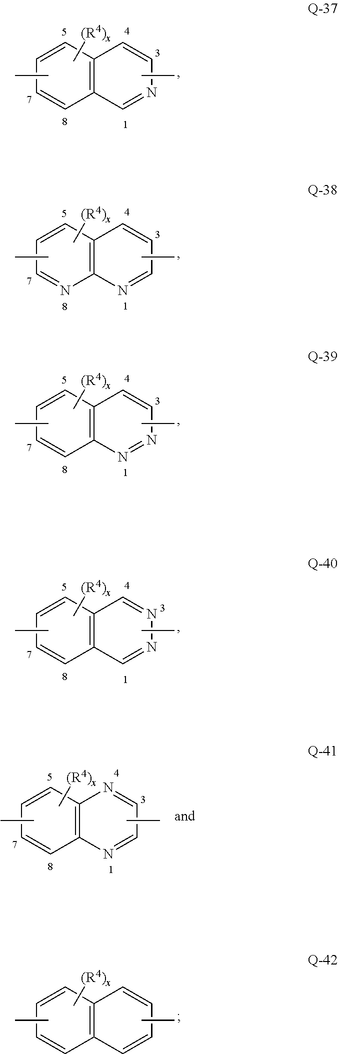

wherein one of the floating bonds is connected to SO₂ in Formula 1 through any available carbon or nitrogen atom of the depicted ring or ring system and the other floating bond is connected to L in Formula 1 through any available carbon or nitrogen atom of the depicted ring or ring system; when R⁴ is attached to a carbon ring member, said R⁴ is selected from R^{4a}, and when R⁴ is attached to a nitrogen ring member, said R⁴ is selected from R^{4b}; and x is an integer from 0 to 5.

### Embodiment 2

A compound of Embodiment 1 wherein Q is a ring selected from the group consisting of Q-4, Q-5, Q-12, Q-20, Q-22, and Q-24.

### Embodiment 3

A compound of Embodiment 2 wherein Q is selected from Q-4, Q-20 and Q-24.

### Embodiment 4

A compound of Embodiment 3 wherein Q is Q-4.

### Embodiment 5

A compound of Embodiment 3 wherein Q is Q-20.

### Embodiment 6

A compound of Embodiment 3 wherein Q is Q-24.

### Embodiment 6a

A compound of Embodiment 6 wherein the SO₂ and L groups connecting Q-24 to the remainder of Formula 1 are attached para relative to each other.

### Embodiment 6b

A compound of Embodiment 6 wherein the SO₂ and L groups connecting Q-24 to the remainder of Formula 1 are attached meta relative to each other.

### Embodiment 7

A compound of Formula 1 or any one of Embodiments 1 through 6b wherein x is 0, 1, 2 or 3.

### Embodiment 8

A compound of Embodiment 7 wherein x is 0 or 1.

### Embodiment 9

A compound of Embodiment 8 wherein x is 0.

### Embodiment 10

A compound of Embodiment 8 wherein x is 1.

### Embodiment 11

A compound of Formula 1 or any one of Embodiments 1 through 10 wherein L is O.

### Embodiment 12

A compound of Formula 1 or any one of Embodiments 1 through 10 wherein L is (CR^{13a}R^{13b})ₜ, CR¹⁴═CR¹⁴, C≡C, CR^{15a}R^{15b}X, XCR^{15a}R^{5b}, CO, S(O)ₚ, NR⁵, CONR⁵NR⁵CO, NR⁵SO₂ or SO₂NR⁵.

### Embodiment 12a

A compound of Embodiment 12 wherein L is (CR^{13a}R^{13b})ₜ, C≡C, CR^{15a}R^{15b}X or XCR^{15a}R^{15b}.

### Embodiment 13

A compound of Embodiments 12 or 12a wherein L is (CR^{13a}R^{13b})ₜ, C≡C or CR^{15a}R^{15b}X.

### Embodiment 13a

A compound of Embodiments 12a or 13 wherein L is (CR^{13a}R^{13b})ₜ.

### Embodiment 13b

A compound of Embodiments 12a or 13 wherein L is C≡C.

### Embodiment 13c

A compound of Embodiments 12a or 13 wherein L is CR^{15a}R^{15b}X.

### Embodiment 13d

A compound of Embodiments 12a or 13 wherein L is XCR^{15a}R^{15b}.

### Embodiment 14

A compound of Formula 1 or any one of Embodiments 1 through 10 and 12 through 13a wherein t is 1.

### Embodiment 15

A compound of Formula 1 or any one of Embodiments 1 through 10 and 12, 12a, 13, 13a and 14 wherein each R^{13a} is independently hydrogen, halogen or C₁-C₂ alkyl.

### Embodiment 16

A compound of Formula 1 or any one of Embodiments 1 through 10, 12, 12a, 13, 13c and 13d wherein X is O.

### Embodiment 17

A compound of Formula 1 or any one of Embodiments 1 through 16 wherein each R¹ is independently halogen, cyano, nitro, OR⁶, C₁-C₃ alkyl or C₁-C₃ haloalkyl.

### Embodiment 18

A compound of Embodiment 17 wherein each R¹ is independently fluorine, chlorine, CH₃, CF₃, OCF₃ or OCHF₂.

### Embodiment 19

A compound of Formula 1 or any one of Embodiments 1 through 18 wherein n is 0, 1 or 2.

### Embodiment 20

A compound of Embodiment 19 wherein n is 0.

### Embodiment 21

A compound of Formula 1 or any one of Embodiments 1 through 20 wherein R² is hydrogen, C₁-C₆ alkyl, C₁-C₆ haloalkyl, C₂-C₆ alkenyl, C₂-C₆ haloalkenyl or C₂-C₆ alkynyl.

### Embodiment 22

A compound of Embodiment 21 wherein R² is hydrogen or methyl.

### Embodiment 23

A compound of Embodiment 22 wherein R² is hydrogen.

### Embodiment 24

A compound of Formula 1 or any one of Embodiments 1 through 23 wherein each R³ is independently halogen, cyano, nitro, OR⁶, NR^{7a}R^{7b}, C(O)R⁸, C(O)OR⁹, C(O)NR¹⁰R¹¹, S(O)ₚR¹², S(O)₂NR¹⁰R¹¹, C₁-C₆ alkyl or C₁-C₆ haloalkyl.

### Embodiment 24a

A compound of Embodiment 24 wherein each R³ is independently cyano, nitro, OR⁶, NR^{7a}R^{7b}, C(O)R⁸, C(O)OR⁹, C(O)NR¹⁰R¹¹, S(O)ₚR¹², S(O)ₚNR¹⁰R¹¹, C₅-C₆ alkyl or C₁-C₆ haloalkyl.

### Embodiment 25

A compound of Embodiment 24 wherein each R³ is independently halogen, cyano, OR⁶, S(O)ₚR¹², C₁-C₄ alkyl or C₁-C₄ haloalkyl.

### Embodiment 26

A compound of Formula 1 or any one of Embodiments 1 through 25 wherein m is 0, 1 or 2.

### Embodiment 27

A compound of Embodiment 26 wherein m is 0.

### Embodiment 28

A compound of Formula 1 or any one of Embodiments 1 through 27 wherein each R^{4a} is independently halogen, cyano, nitro, OR⁶, C₁-C₆ alkyl or C₁-C₆ haloalkyl.

### Embodiment 29

A compound of Formula 1 or any one of Embodiments 1 through 27 wherein R^{4b} is methyl.

### Embodiment 30

A compound of Formula 1 or any one of Embodiments 1 through 29 wherein R⁵ is hydrogen or C₁-C₆ alkyl.

### Embodiment 31

A compound of Formula 1 or any one of Embodiments 1 through 30 wherein each R⁶ is independently hydrogen, C₁-C₆ alkyl or C₁-C₆ haloalkyl.

### Embodiment 31a

A compound of Embodiment 31 wherein each R⁶ is independently hydrogen, C₅-C₆ alkyl and C₂-C₆ haloalkyl.

### Embodiment 32

A compound of Embodiment 31 wherein each R⁶ is independently hydrogen, C₁-C₂ alkyl or C₁-C₂ haloalkyl.

### Embodiment 33

A compound of Formula 1 or any one of Embodiments 1 through 32 wherein each R^{7a} is independently hydrogen, C₁-C₆ alkyl or C₁-C₆ haloalkyl.

### Embodiment 34

A compound of Embodiment 33 wherein each R^{7a} is independently hydrogen, C₁-C₂ alkyl or C₁-C₂ haloalkyl.

### Embodiment 35

A compound of Formula 1 or any one of Embodiments 1 through 34 wherein each R^{7b} is independently hydrogen, C₁-C₂ alkyl or C1-C₂ haloalkyl.

Embodiments of this invention, including Embodiments 1-35 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-35 above as well as any other embodiments described herein, and any combination thereof, pertain to the compositions and methods of the present invention.

Combinations of Embodiments 1-35 are illustrated by:

### Embodiment AAA

A compound of Formula 1 wherein
   Q is phenyl or naphthalenyl each optionally substituted with up to 5 substituents independently selected from R^{4a}; or
   Q is a 5- to 6-membered heteroaromatic ring or an 8- to 11-membered heteroaromatic bicyclic ring system, each ring or ring system containing ring members selected from carbon atoms and up to 4 heteroatoms independently selected from up to 2 O, up to 2 S and up to 4 N atoms, and optionally substituted with up to 5 substituents independently selected from R^{4a} on carbon atom ring members and R^{4b} on nitrogen atom ring members;
   L is (CR^{13a}R^{13b})ₜ, CR¹⁴═CR¹⁴, C≡C, CR^{15a}R^{15b}X, XCR^{15a}R^{15b}, CO, O, S(O)ₚ, NR⁵, CONR⁵, NR⁵CO, NR⁵SO₂ or SO₂NR⁵;
   X is O, S, SO, SO₂, or NR⁵;
   each R¹ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   R² is hydrogen, cyano, OR⁶, NR^{7a}R^{7b}, C(O)R⁸, C(O)OR⁹, C(O)NR¹⁰R¹¹, S(O)ₚR¹²or S(O)₂NR¹⁰R¹¹; or C₁-C₆ alkyl, C₂-C₆ alkenyl, C₁-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   each R³ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   each R^{4a} is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   R^{4b} is cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR²;
   R⁵ is hydrogen, cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   each R⁶ is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl and S(O)ₚR¹²;
   each R^{7a} is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   each R^{7b} is independently hydrogen; or C₁-C₆ alkyl, C₂-C₆ alkenyl, C₁-C₆ alkynyl or benzyl each optionally substituted with substituents independently selected from the group consisting of halogen, cyano, nitro, OR⁶, C(O)R⁸, C(O)OR⁹, C(O)NR¹⁰R¹¹, S(O)ₚR¹² and S(O)₂NR¹⁰R¹¹;
   R⁸, R⁹, R¹⁰ and R¹² are each independently hydrogen; or C₁-C₆ alkyl, C₁-C₆ alkenyl, C₂-C₆ alkynyl, phenyl, benzyl, C₃-C₇ cycloalkyl, C₄-C₈ cycloalkylalkyl or C₅-C₇ cycloalkenyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₂-C₆ alkoxycarbonyl, C₂-C₆ alkylaminocarbonyl, C₂-C₈ dialkylaminocarbonyl, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl and C₁-C₄ haloalkylsulfonyl;
   each R¹¹ is independently hydrogen; or C₁-C₆ alkyl, C₂-C₆ alkenyl, C₁-C₆ alkynyl or benzyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl and C₁-C₄ haloalkylsulfonyl;
   each R^{13a} is independently hydrogen, halogen, cyano, hydroxyl or amino; or C₁-C₆ alkoxycarbonyl, C₂-C₆ alkylaminocarbonyl or C₂-C₈ dialkylaminocarbonyl; or C₁-C₆ alkyl, C₂-C₆ alkenyl, C₁-C₆ alkynyl, C₃-C₇ cycloalkyl, C₄-C₈ cycloalkylalkyl, phenyl or benzyl each optionally substituted with substituents independently selected from the group consisting of halogen, cyano, nitro, C₁-C₄ alkyl, C₁-C₄ alkoxy, C₁-C₄ haloalkyl, C₁-C₄ haloalkoxy, C₂-C₆ alkoxycarbonyl, C₂-C₆ alkylaminocarbonyl and C₂-C₈ dialkylaminocarbonyl;
   each R^{13b} is independently hydrogen, halogen, or C₁-C₆ alkyl;
   each R¹⁴ is independently hydrogen, halogen, cyano, C₁-C₆ alkyl or C₁-C₆ haloalkyl;
   R^{15a} is hydrogen, cyano, C₁-C₆ alkyl, C₁-C₆ haloalkyl, C₂-C₆ alkenyl, C₂-C₆ haloalkenyl, C₂-C₆ alkynyl, C₁-C₆ haloalkynyl, C₃-C₇ cycloalkyl, C₄-C₈ cycloalkylalkyl, C₅-C₇ cycloalkenyl, phenyl, benzyl, C₂-C₆ alkoxycarbonyl, C₂-C₆ alkylaminocarbonyl or C₂-C₈ dialkylaminocarbonyl;
   R^{15b} is hydrogen or C₁-C₆ alkyl;
   n is 0, 1, 2, 3, 4 or 5;
   m is 0, 1, 2, 3, 4 or 5;
   p is 0, 1 or 2; and
   t is 1, 2 or 3;
   provided that when
   L is an oxygen atom, then one R³ group is other than hydrogen, halogen, C₁-C₄ alkyl, C₁-C₄ alkoxy, C₁-haloalkyl or C₁-haloalkoxy.

### Embodiment AA

A compound of Embodiment AAA or a compound of Formula 1 as described in the summary of the invention wherein
   Q is phenyl or naphthalenyl each optionally substituted with up to 5 substituents independently selected from R^{4a}; or
   Q is a 5- to 6-membered heteroaromatic ring or an 8- to 11-membered heteroaromatic bicyclic ring system, each ring or ring system containing ring members selected from carbon atoms and up to 4 heteroatoms independently selected from up to 2 O, up to 2 S and up to 4 N atoms, and optionally substituted with up to 5 substituents independently selected from R^{4a} on carbon atom ring members and R^{4b} on nitrogen atom ring members;
   L is (CR^{13a}R^{13b})₁, CR¹⁴═CR¹⁴, C≡C, CR^{15a}R^{15b}X, XCR^{15a}R^{15b}, CO, O, S(O)ₚ, NR⁵, CONR⁵, NR⁵CO, NR⁵SO₂ or SO₂NR⁵;
   X is O, S, SO, SO₄, or NR⁵;
   each R¹ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   R² is hydrogen, cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   each R³ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   each R^{4a} is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   R^{4b} is cyano, OR⁶, NR^{7a}R^{7b}, C(O)R⁸, C(O)OR⁹, C(O)NR¹⁰R¹¹, S(O)ₚR² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)₂R¹² and S(O)ₚNR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR²;
   R⁵ is hydrogen, cyano, OR⁶, NR^{7a}R^{7b}, C(O)R⁸, C(O)OR⁹, C(O)NR¹⁰R¹¹, S(O)ₚR¹² or S(O)₂NR¹⁰R^{7b}; or C₁-C₆ alkyl, C₂-C₆ alkenyl, C₁-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)R¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
   each R⁶ is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, C₁-C₆ alkoxy and S(O)ₚR¹²;
   each R^{7a} is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, C₁-C₆ alkoxy and S(O)ₚR²;
   each R^{7b} is independently hydrogen; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹;
   R⁸, R⁹, R¹⁰ and R¹² are each independently hydrogen; or C₁-C₆ alkyl, C₁-C₆ alkenyl, C₂-C₆ alkynyl, phenyl, benzyl, C₃-C₇ cycloalkyl, C₄-C₈ cycloalkylalkyl or C₅-C₇ cycloalkenyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₁-C₆ alkoxycarbonyl, C₂-C₆ alkylaminocarbonyl, C₂-C₈ dialkylaminocarbonyl, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl and C₁-C₄ haloalkylsulfonyl;
   each R¹¹ is independently hydrogen; or C₁-C₆ alkyl, C₂-C₆ alkenyl, C₂-C₆ alkynyl or benzyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl and C₁-C₄ haloalkylsulfonyl;
   each R^{13a} is independently hydrogen, halogen, cyano, hydroxyl or amino; or C₂-C₆ alkoxycarbonyl, C₂-C₆ alkylaminocarbonyl or C₂-C₈ dialkylaminocarbonyl; or C₁-C₆ alkyl, C₂-C₆ alkenyl, C₂-C₆ alkynyl, C₃-C₇ cycloalkyl, C₄-C₈ cycloalkylalkyl, phenyl or benzyl each optionally substituted with substituents independently selected from the group consisting of halogen, cyano, nitro, C₁-C₄ alkyl, C₁-C₄ alkoxy, C₁-C₄ haloalkyl, C₁-C₄ haloalkoxy, C₂-C₆ alkoxycarbonyl, C₁-C₆ alkylaminocarbonyl and C₂-C₈ dialkylaminocarbonyl;
   each R^{13b} is independently hydrogen, halogen, or C₁-C₆ alkyl;
   each R¹⁴ is independently hydrogen, halogen, cyano, C₁-C₆ alkyl or C₁-C₆ haloalkyl;
   R^{15a} is hydrogen, cyano, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₇ cycloalkyl, C₄-C₈ cycloalkylalkyl, C₅-C₇ cycloalkenyl, phenyl, benzyl, C₂-C₆ alkoxycarbonyl, C₂-C₆ alkylaminocarbonyl or C₂-C₈ dialkylaminocarbonyl;
   R^{15b} is hydrogen or C₁-C₆ alkyl;
   n is 0, 1, 2, 3, 4 or 5;
   m is 0, 1, 2, 3, 4 or 5;
   p is 0, 1 or 2; and
   t is 1, 2 or 3;
   provided that when
   L is an oxygen atom, then one R³ group is other than hydrogen, halogen, C₁-C₄ alkyl, C₁-C₄ alkoxy, C₁-haloalkyl or C₁-haloalkoxy.

### Embodiment A

A compound of Embodiment AA wherein
   Q is a ring selected from the group consisting of Q-1 through Q-42 in Exhibit 1 wherein one of the floating bonds is connected to SO₂ in Formula 1 through any available carbon or nitrogen atom of the depicted ring or ring system and the other floating bond is connected to L in Formula 1 through any available carbon or nitrogen atom of the depicted ring or ring system; when R⁴ is attached to a carbon ring member, said R⁴ is selected from R^{4a}, and when R⁴ is attached to a nitrogen ring member, said R⁴ is selected from R^{4b}; and x is an integer from 0 to 5;
   L is (CR^{13a}R^{13b})ₜ, C≡C or CR^{15a}R^{15b}X;
   X is O;
   each R¹ is independently halogen, cyano, nitro, OR⁶, C₁-C₃ alkyl or C₁-C₃ haloalkyl;
   each R^{4a} is independently halogen, cyano, nitro, OR⁶, C₁-C₆ alkyl or C₁-C₆ haloalkyl;
   R^{4b} is methyl; and
   n is 0, 1 or 2.

### Embodiment B

A compound of Embodiment A wherein
   Q is Q-24;
   x is 0, 1, 2 or 3;
   L is (CR^{13a}R^{13b})ₜ;
   t is 1;
   each R^{13a} is independently hydrogen, halogen or C₁-C₂ alkyl;
   R² is hydrogen or methyl;
   each R³ is independently halogen, cyano, nitro, OR⁶, NR^{7a}R^{7b}, C(O)R⁸, C(O)OR⁹, C(O)NR¹⁰R¹¹, S(O)ₚR¹², S(O)₂NR¹⁰R¹¹, C₁-C₆ alkyl or C₁-C₆ haloalkyl;
   each R⁶ is independently hydrogen, C₁-C₆ alkyl or C₁-C₆ haloalkyl; and
   m is 0, 1 or 2.

### Embodiment C

A compound of Embodiment B wherein
   each R¹ is independently fluorine, chlorine, CH₃, CF₃, OCF₃ or OCHF₂;
   R² is hydrogen; and
   each R³ is independently halogen, cyano, OR⁶, S(O)ₚR¹², C₁-C₄ alkyl or C₁-C₄ haloalkyl.

### Embodiment D

A compound of Formula 1 wherein
   Q is Q-24;
   x is 0, 1, 2 or 3;
   L is O;
   each R¹ is independently halogen, cyano, nitro, OR⁶, C₁-C₃ alkyl or C₁-C₃ haloalkyl;
   R² is hydrogen or methyl;
   each R³ is independently cyano, nitro, OR⁶, NR^{7a}R^{7b}, C(O)R⁸, C(O)OR⁹, C(O)NR¹⁰R¹⁰R¹¹, S(O)ₚR¹², S(O)₂NR¹⁰R¹¹, C₅-C₆ alkyl or C₂-C₆ haloalkyl.
   each R^{4a} is independently halogen, cyano, nitro, OR⁶, C₁-C₆ alkyl or C₁-C₆ haloalkyl;
   each R⁶ is independently hydrogen, C₅-C6 alkyl and C₂-C₆ haloalkyl;
   n is 0, 1 or 2; and
   m is 0, 1 or 2.

Specific embodiments include compounds of Formula 1 selected from the group consisting of:
4-(3-chloro-2-cyanophenoxy)-N-(4-quinolinylmethyl)benzenesulfonamide;
4-(phenylmethyl)-N-(4-quinolinylmethyl)benzenesulfonamide;
4-(4-cyanophenoxy)-N-(4-quinolinylmethyl)benzenesulfonamide;
4-(2-chloro-4-cyanophenoxy)-N-(4-quinolinylmethyl)benzenesulfonamide;
4-(2-cyanophenoxy)-N-(4-quinolinylmethyl)benzenesulfonamide;
4-(phenoxymethyl)-N-(4-quinolinylmethyl)benzenesulfonamide;
4-[2-(4-chlorophenyl)ethynyl]-N-(4-quinolinylmethyl)benzenesulfonamide;
4-(3-cyanophenoxy)-N-(4-quinolinylmethyl)benzenesulfonamide;
4-[2-(2,4-dichlorophenyl)ethynyl]-N-(4-quinolinylmethyl)benzenesulfonamide; and
4-[(4-chlorophenoxy)methyl]-N-(4-quinolinylmethyl)benzenesulfonamide.

Another, specific embodiment includes the compound of Formula 1,
4-[(4-fluorophenyl)methyl]-N-(4-quinolinylmethyl)benzenesulfonamide.

Also noteworthy as embodiments of the present invention are compositions comprising a compound of any of the preceding Embodiments, as well as any other embodiments described herein, and their use for treating, controlling, preventing or protecting animals against infection by helminths.

Also noteworthy as embodiments of the present invention are compositions comprising a compound of any of the preceding Embodiments, as well as any other embodiments described herein, in a parasiticidally effective amount and at least one pharmaceutically or veterinarily acceptable carrier or diluent.

Further noteworthy as embodiments of the present invention are compositions comprising a compound of any of the preceding Embodiments, as well as any other embodiments described herein, and at least one pharmaceutically or veterinarily acceptable carrier or diluent, said composition further comprising at least one additional biologically active compound or agent.

Embodiments of the invention also include an anthelmintic composition comprising a mixture of a compound of Formula 1 (including all stereoisomers) or an N-oxide or salt thereof and at least one other anthelmintic (e.g., at least one other anthelmintic having a different site of action).

Embodiments of the invention also include a method for treating, controlling, preventing or protecting animals against infection by helminths which comprises administration enterally, for example orally, parenterally, for example by injection, (including subcutaneously, intramuscularly or intravenously) or topically, to the animal, of a parasiticdally effective amount of a compound of Formula 1 (including all stereoisomers) or an N-oxide, or a pharmaceutically or veterinarily acceptable salt or a composition comprising it.

Embodiments of the invention also include a method for treating, controlling, preventing or protecting an animal against infection by helminths wherein the animal is a human.

Embodiments of the invention also include a method for treating, controlling, preventing or protecting animals against infection by helminths wherein the animal is non-human.

Embodiments of the invention also include a method for treating, controlling, preventing or protecting animals against infection by helminths wherein the helminth is a nematode.

Embodiments of the invention also include a method for controlling parasitic worms comprising administration enterally for example orally, parenterally, for example by injection, (including subcutaneously, intramuscularly or intravenously or topically, of a parasiticidally effective amount of Formula 1 (including all stereoisomers) or an N-oxide or salt thereof (e.g., as a composition described herein) Embodiments of the invention also include methods for controlling helminths comprising contacting the helminth or its environment with a parasiticidally effective amount of a compound of Formula 1, an N-oxide, or a salt thereof, (e.g., as a composition described herein), provided that the methods are not methods of medical treatment of a human or animal body by therapy.

Embodiments of the invention also include a compound of Formula 1 (including all stereoisomers) or an N-oxide or salt thereof, or any of the preceding Embodiments for use as an animal medicament, or more particularly a parasiticidal animal medicament. The medicament may be in any art recognized dosage forms including oral, topical, parenteral or subcutaneous dosage forms.

Embodiments of the invention also include a compound of Formula 1 (including all stereoisomers) or an N-oxide or salt thereof, or any of the preceding Embodiments for the manufacture of a medicament for the protection of an animal from a helminth. The medicament may be in any art recognized dosage forms including oral, topical, parenteral or subcutaneous dosage forms.

Embodiments of the invention also include a compound of Formula 1 (including all stereoisomers) or an N-oxide or salt thereof, or any of the preceding Embodiments, packaged and presented for the protection of an animal from a helminth. The compounds of the invention may be packaged and presented as in any dosage form suitable for the mode of intended administration.

Embodiments of the invention also include a process for manufacturing a composition for protecting an animal from a helminth characterized as a compound of Formula 1 (including all stereoisomers) or an N-oxide or salt thereof, or any of the preceding Embodiments, admixed with at least one carrier or diluent. The compounds of the invention may be packaged and presented in any art recognized dosage forms including oral, topical, parenteral or subcutaneous dosage forms.

One or more of the following methods and variations as described in Schemes 1-5 and the reactions of entries 1-14 in Table A can be used to prepare the compounds of Formulae 1. The definitions of Q, L, R¹, R² and R³ in the compounds of Formulae 1-46 and Formulae 1a-1b are as defined above in the Summary of the Invention unless otherwise noted. Formulae 1a-1b are subsets of Formula 1, and all substituents for Formulae 1a-1b are as defined above for Formula 1 unless otherwise noted. Ambient or room temperature is defined as about 20-25° C.

Compounds of Formula 1 can be prepared by the reaction of 4-quinolinemethanamines of Formula 2 with aryl or heteroaryl sulfonylchlorides of Formula 3, typically in the presence of base, as shown in Scheme 1. The reaction can be carried out at temperatures ranging from 0° C. to the reflux temperature of the solvent, preferably in the range of room temperature to 100° C. Typical solvents include aliphatic and aromatic hydrocarbons such as hexane or toluene; ethers such as diethyl and diisopropyl ether, tetrahydrofuran or dioxane; esters such as ethyl acetate; nitriles such as acetonitrile; ketones such as acetone or methyl ethyl ketone; amides such as dimethylformamide and dimethylacetamide; and halogenated hydrocarbons such as methylene chloride and chloroform. Typical bases for the reaction include pyridine and substituted pyridines such as the picoline isomers, trialkylamines such as triethyl, tributyl or diisopropylethylamine, and metal carbonates such sodium or potassium carbonate.

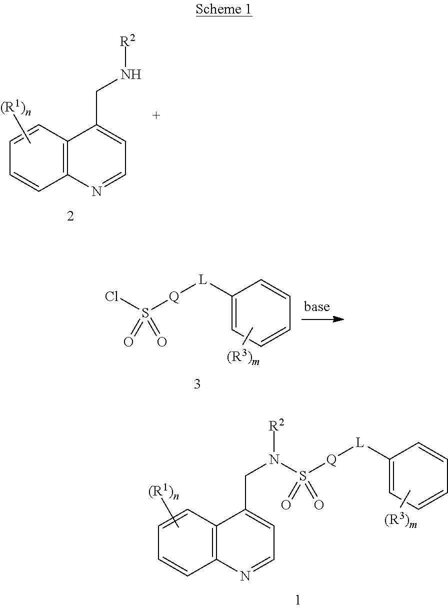

Compounds of Formula 1, where R² is e.g. alkyl, substituted alkyl, acyl, sulfonyl and the like, may also be prepared by the reaction of quinoline sulfonamides of Formula 4 with various alkylating, acylating or sulfonylating reagents, such as R²—X, in the presence of a base, as shown in Scheme 2. Typical bases include pyridine and substituted pyridines such as the picoline isomers; trialkylamines such as triethyl, tributyl or diisopropylethylamine; hydrides such as sodium hydride; and carbonates such potassium or cesium carbonate. Typical solvents include acetonitrile, tetrahydrofuran, dimethylformamide, dimethylacetamide, ethyl acetate, and toluene. The reaction is typically run at room temperature but may be carried out at temperatures ranging from room temperature to the reflux temperature of the solvent.

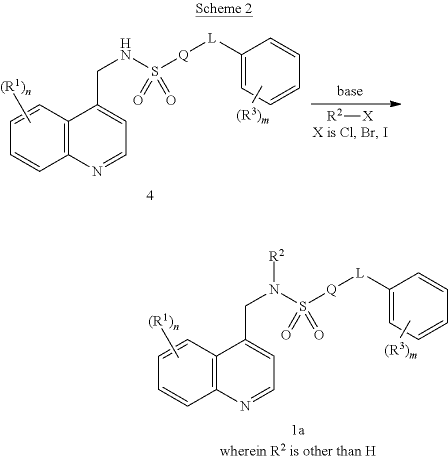

A variety of the intermediate sulfonyl chlorides of Formula 3 are known or are available from commercial sources. Intermediate sulfonyl chlorides of Formula 3 may also be prepared by a wide variety of well known methods. One particularly useful method is by the diazotization and chlorosulfonation of aromatic and heteroaromatic amines of Formula 6. These methods and procedures are extensively documented in the chemical literature. A typical set of conditions includes sodium nitrite, copper chloride, and sulfur dioxide in a mixture of acetic and hydrochloric acid. Details can be found in Example 1 Step C. Experimental details using thionyl chloride as the sulfonyl chloride source can be found in Examples 2 and 3, Step C. The amines of Formula 6 are readily available from a variety of sources with the reduction of aromatic and heteroaromatic nitro compounds of Formula 5 being very typical.

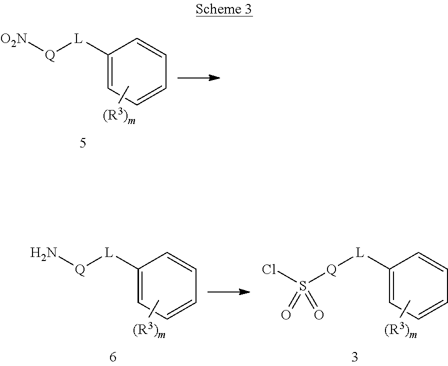

An alternative useful procedure for the preparation of the intermediate sulfonyl chlorides of Formula 3 is by the oxidative chlorination of sulfides to the corresponding sulfonyl chlorides as shown in Scheme 4. Treatment of sulfides of Formula 8 with chlorinating reagents including chlorine, N-chlorosuccinimide, and sodium hypochlorite provides the corresponding sulfonyl chlorides of Formula 3 under a wide range of conditions (see e.g. World Patent Publication WO2007/147762, Tetrahedron Lett. 2010, 51 418-421). The intermediate sulfides 8 are available from aryl or heterocyclic halides of Formula 7 by displacement with benzyl mercaptan by a variety of known literature procedures.

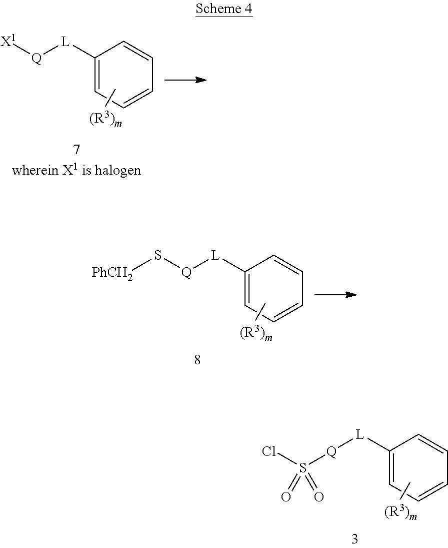

A variety of intermediates of Formula 5, 7 and 8 represented by the various L groups of Formula 1, as well as analogs of these intermediates, are known or are available commercially. Further, intermediates of Formula 5, 7, and 8 are available by many conventional synthetic methods. The L groups found within compounds of Formula 1 and within intermediates of Formula 5, 7, and 8 can be introduced by known general synthetic reactions useful for the preparation of the various L groups. Typical examples of these reactions can be found in Table A. In the entries of Table A the group J represents a substituent of a functionalized aromatic Q group such as those of Formulae 5, 7 and 8 (e.g. halogen, nitro, benzylsulfide) or derivatives of these compounds that are readily transformed to these groups by procedures well known in the art.

TABLE A
TABLE A Reaction Summary

| | | | |
|---|---|---|---|
| 1 L is O | 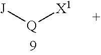 | 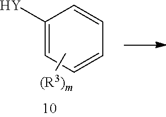 | 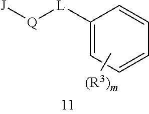 |
| | wherein X¹ is halogen | Y is O or S | L is O or S |
| 2 L is SO, SO₂ | 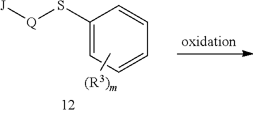 | 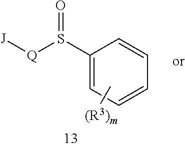 | 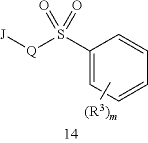 |
| | | wherein L is SO | L is SO₂ |
| 3 L is CH₂ | 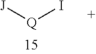 | 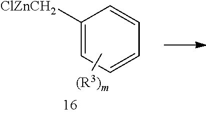 | 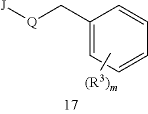 |
| | | | wherein L is CH₂ |
| 4 L is CH₂Y | 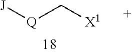 | 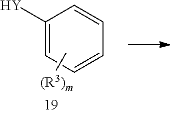 | 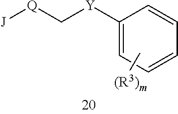 |
| | wherein X¹ is halogen | Y is O or S | L is CH₂Y and Y is O or S |
| 5 L is YCH₂ | 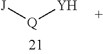 | 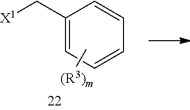 | 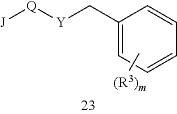 |
| | wherein Y is O or S | X¹ is halogen | L is YCH₂ and Y is O or S |
| 6 L is C≡C | 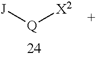 | 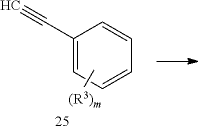 | 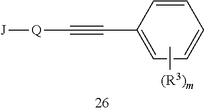 |
| | wherein X² is Br or I | | L is C≡C |
| 7 L is CH═CH | 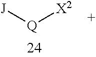 | 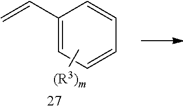 | 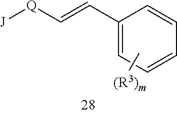 |
| | wherein X² is Br or I | X¹ is halogen | L is CH═CH |
| 8 L is CH₂CH₂ | 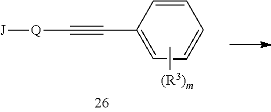 | 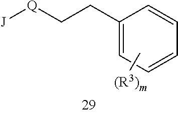 | |
| | | wherein L is CH₂CH₂ | |
| 9 L is C(O), CH₂OH | 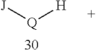 | 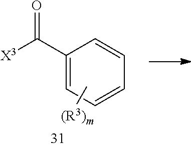 | 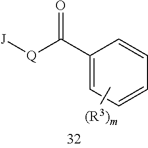 |
| | | wherein X³ is Cl or OH | L is C(O) |
| | 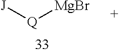 | 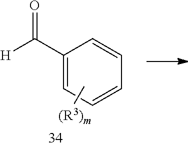 | 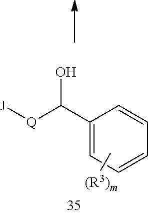 |
| | | | wherein L is CH(OH) |
| 10 L is NR⁵C(O) | 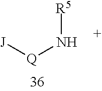 | 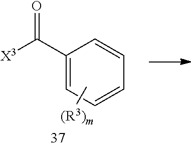 | 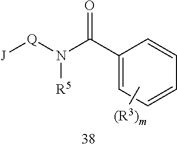 |
| | | wherein X³ is Cl or OH | L is NR⁵C(O) |
| 11 L is C(O)NR⁵ | 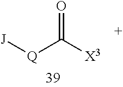 | 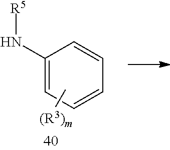 | 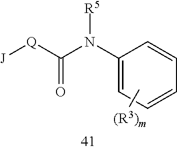 |
| | wherein X³ is Cl or OH | | L is C(O)NR⁵ |
| 12 L is NR⁵SO₂ | 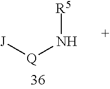 | 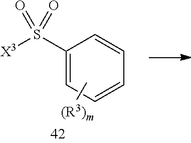 | 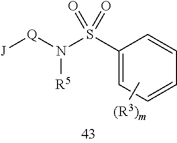 |
| | | wherein X⁴ is Cl | L is NR⁵SO₂ |
| 13 L is SO₂NR⁵ | 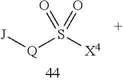 | 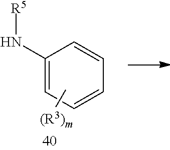 | 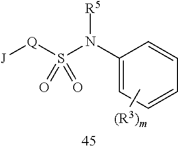 |
| | wherein X⁴ is Cl | | L is SO₂NR⁵ |

1. Many aryl ethers and thioethers of Formula 11 may be prepared by the reaction of phenols and thiophenols of Formula 10 with aromatic halides of Formula 9 in the presence of a base such as potassium carbonate or sodium hydride and in solvents such as DMF, DMA or THF (World Patent Publication WO 2005/023242, World Patent Publication WO 2005/074375 and World Patent Publication WO 2007/064045).
2. Aryl sulfinyl ethers of Formula 13 are readily prepared by oxidation of sulfides of Formula 12 with o-Me-IBX (Tetrahedron Letters 2007, 49, 80-84). Sulfonyl derivatives of Formula 14 can be prepared by the oxidation of aryl sulfides of Formula 12 with a wide variety of reagents including potassium permanganate (World Patent Publication WO 2006/100519) and Oxone® (Organic Process Research & Development 2009 13, 456-462).
3. Synthesis of Formula 17 intermediates where L is a methylene group can be prepared by the palladium catalyzed coupling of benzylzine derivatives of Formula 16 with aromatic halides of Formula 15 (J. Med. Chem. 2009 52, 4869-4882; World Patent Publication WO 2005/113509).
4. The preparation of alkyl ether and alkylthio ether intermediates of Formula 20 may be prepared by a wide variety of methods typical for the Williamson ether synthesis, generally employing the alkylation of a benzylic halide of Formula 18 with an aromatic ether or thioether of Formula 19.
5. The preparation of alkyl ether and alkylthio ether intermediates of Formula 23 may also be prepared by a wide variety of methods typical for the Williamson ether synthesis generally employing the alkylation of a benzylic halide of Formula 22 with an aromatic ether or thioether of Formula 21.
6. Acetylene derivatives of Formula 26 may be prepared by the palladium catalyzed coupling of an aryl acetylene of Formula 25 with an arylhalide of Formula 24 under a variety of known conditions for the Sonogoshira reaction (J. Am. Chem. Soc. 2010, 132, 9585-9587; U.S. Pat. No. 7,642,391, J. Org. Chem. 2006, 71, 9499-9502 and J. Org. Chem. 2005, 70, 4393-4396).
7. Styrene intermediates of Formula 28 can be prepared by a Heck or modified Heck reaction involving the palladium catalyzed cross coupling of styrene derivatives of Formula 27 with aromatic and heteroaromatic halides of Formula 24 (Bioorganic & Medicinal Chemistry 2010, 18, 5352-5366; Journal of Oganometallic Chemistry 2010, 695, 2284-2295; Tetrahedron Letters 2004, 45, 7689-7691).
8. Alkylene derivatives of Formula 29 are available by catalytic hydrogenation of the arylacetylenes or arylstyrenes of Formulae 26 and 28 respectively.
9. Ketone derivatives of Formula 32 can be prepared by a variety of methods including Friedel Crafts acylation of the aromatic derivatives of Formula 30 with acids or acid halides of Formula 31. Carbinols of Formula 35 can be prepared by the addition of an aryl Grignard of Formula 33 to an aryl aldehyde of Formula 34. The Formula 35 carbinols offer an alternative synthesis of the ketones of Formula 32 by oxidation.
10-13. Amide and sulfonamide intermediates of Formulae 38, 41, 43 and 45 found in Entries 10-13 are available by well known methods involving the coupling of anilines of Formulae 36 and 40 with acyl chlorides of Formulae 37 and 39 and sulfonyl chlorides of Formulae 42 and 44 respectively.

The quinolines of Formula 2 are known in the literature or can be prepared by a variety of methods from the intermediates of Formula 46a-46d (World Patent Publication WO 2007/052262) shown in Scheme 5. Oximes of Formula 46a can be readily reduced to the amines of Formula 2 (R² is H). A specific procedure with palladium and ammonium formate in methanol is described in Example 1. Other methods for this reduction can be found in the following references: J. Org. Chem. 1989, 54, 1731-5 and European Patent Publication EP 1571150. The R² groups of Formula 2 may be introduced by reductive amination, or alkylation reactions. The oximes of Formula 46a are available from the corresponding aldehydes of Formula 46b by treatment with hydroxylamine. Aldehydes of Formula 46b can be prepared from the corresponding bromo derivatives 46d by a variety of methods including metal halogen exchange and treatment with dimethylformamide. See for example J. Med. Chem. 2009, 52, 6966-6978; Bioorganic & Medicinal Chemistry Letters 2010, 20, 1347-1351 and J. Med. Chem. 2009, 52, 6966-6978.

The quinolines of Formula 2 can also be prepared from nitriles of Formula 46c by catalytic hydrogenation. References applicable to this transformation include the following: World Patent Publication WO 2008/007211, World Patent Publication WO 2008/090434, World Patent Publication WO 2007/104726, and World Patent Publication WO 2008/079292. The nitriles 46c can be prepared from the corresponding bromo derivatives 46d by reaction with a cyanide source. See for example Organic Letters 2007, 9, 5525-5528; J. Med. Chem. 1992, 35, 2761-8; Bioorganic & Medicinal Chemistry Letters 2005, 15, 4520-4525.

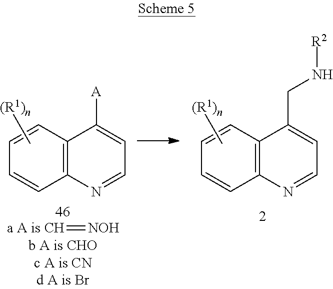

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Ambient or room temperature is defined as about 20-25° C. Percentages are by weight except for chromatographic solvent mixtures or where otherwise indicated. Parts and percentages for chromatographic solvent mixtures are by volume unless otherwise indicated. MPLC refers to medium pressure liquid chromatography on silica gel. ¹H NMR spectra are reported in ppm downfield from tetramethylsilane; “s” means singlet, “d” means doublet, “dd” means doublet of doublets, “ddd” means doublet of doublet of doublets, “t” means triplet, “min” means multiplet, and “br s” means broad singlet. For mass spectral data, the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺).

### SYNTHESIS EXAMPLE 1

### Preparation of 4-(phenylmethyl)-N-(4-quinolinylmethyl)benzenesulfonamide (Compound Number 5)

### Step A: Preparation of 4-quinolinecarboxaldehyde oxime

To 4-quinolinecarboxaldehyde (10.0 g, 62.5 mmol) in 65 mL ethanol was added hydroxylamine HCl (4.81 g, 68.75 mmol) and 3.1 mL water and then pyridine (11.2 mL, 137 mmol) was added dropwise. The reaction mixture was stirred overnight at room temperature. Water (30 mL) was added and the reaction mixture was cooled in an ice bath to precipitate a solid. This solid was filtered and washed with ethanol and water and dried under nitrogen to obtain 11.0 g of the title compound.

¹H NMR (DMSO) δ 12.02 (s, 1H) 8.94 (d, 1H), 8.85 (s, 1H), 8.65 (d, 1H), 8.08 (d, 1H), 7.83 (t, 1H), 7.75 (d, 1H), 7.68 (t, 1H).

### Step B: Preparation of 4-quinolinemethanamine

To a 500 mL round bottom flask under nitrogen was added 10% Pd/C (0.85 g) followed by 4-quinolinecarboxaldehyde oxime (11.0 g, 63 mmol) (i.e. the product of Example 1, Step A) and ammonium formate (16.8 g, 257 mmol). Methanol (200 mL) was carefully added and the reaction mixture was heated to 40-45° C. for 8 hours and then stirred overnight at room temperature. The reaction mixture was then filtered through celite and washed with methanol. The filtrate was then concentrated under reduced pressure to approximately 20 mL and then diluted with 300 mL of methylene chloride and washed with a saturated aqueous sodium carbonate solution (200 mL). The methylene chloride phase was dried over magnesium sulfate and concentrated under reduced pressure to obtain an oil. The oil was chromatographed on silica gel using a gradient of ethyl acetate:methanol (9:1) to pure methanol to provide 6.0 g of the title compound.

¹H NMR (CDCl₃) δ 8.89 (d, 1H), 8.15 (d, 1H), 8.01 (d, 1H), 7.72 (t, 1H), 7.58 (t, 1H), 7.48 (d, 1H), 4.38 (s, 2H).

### Step C: Preparation of 4-(phenylmethyl)benzenesulfonyl chloride

To 4-benzylaniline (2.0 g, 10.9 mmol) in 20 mL of acetic acid and 20 mL of concentrated hydrochloric acid was added sodium nitrite (802 mg, 11.6 mmol) in 5 mL of water dropwise while cooling in an ice bath. After stirring for 45 min this solution was added dropwise to a flask containing copper (1) chloride (0.4 g) and 20 mL acetic acid saturated with sulfur dioxide (3.0 mL). The reaction mixture was then allowed to stir at ambient temperature overnight. The reaction mixture was diluted with 200 mL of ethyl acetate and washed with saturated aqueous sodium bicarbonate solution (3×50 mL). The ethyl acetate phase was separated and dried over magnesium sulfate and then concentrated under reduced pressure to obtain an oil. The crude product was chromatograped on silica gel using a gradient of hexane to hexane:ethyl acetate (95:5) to provide 0.84 g of the title compound as an oil.

¹H NMR (CDCl₃) δ 7.95 (d, 2H), 7.41 (d, 2H), 7.33 (d, 2H), 7.28 (d, 1H), 7.18 (d, 2H), 4.09 (s, 2H).

### Step D: Preparation of 4-(phenylmethyl)-N-(4-quinolinylmethyl)benzenesulfonamide

To 4-quinolinemethanamine (180 mgs, 1.14 mmol) (i.e. the product of Example 1, Step B) in 20 mL tetrahydrofuran and diisopropylamine (440 μl, 2.5 mmol) was added 4-(phenylmethyl)benzenesulfonyl chloride (332 mg, 1.25 mmol) (i.e. the product of Example 1, Step C) and the reaction mixture was stirred at room temperature overnight. The reaction was then concentrated under reduced pressure to obtain an oil which was chromatograped on silica gel using a gradient of hexane:ethyl acetate (6:4) to pure ethyl acetate to provide a residue which was precipitated in diethyl ether and hexane and filtered to obtain 253 mg of the title compound, a compound of the invention, as a white solid, m.p. 106-107° C.

¹H NMR (CDCl₃) 8.77 (d, 1H), 8.10 (d, 1H), 7.87 (d, 1H), 7.79 (d, 2H), 7.70 (t, 1H), 7.53 (t, 1H), 7.33 (m, 4H), 7.27 (m, 2H), 7.18 (d, 1H), 4.95 (t, 1H), 4.62 (d, 2H), 4.05 (s, 2H).

### SYNTHESIS EXAMPLE 2

### Preparation of 4-(2-chloro-4-cyanophenoxy)-N-(quinolinylmethyl)benzenesulfonamide (Compound Number 15)

### Step A: Preparation of 3-chloro 4-(4-nitrophenoxy)benzonitrile

To a solution of 4-fluoronitrobenzene (2.5 g, 17.73 mmol) in dimethylformamide (30 mL) was added 2-chloro-4-cyanophenol (2.7 g, 17.73 mmol) and potassium carbonate (7.34 g, 53.19 mmol). The reaction mixture was stirred at 120° C. for 16 hours in a sealed tube. The reaction mixture was then poured into water and extracted with ethyl acetate. The organic phase was washed with water and saturated aqueous NaCl solution. The ethyl acetate extracts were combined and concentrated under reduced pressure and purified by column chromatography on silica gel (5% EtOAc/hexanes as eluent) to give 3 g of the title compound as a solid.

¹H NMR (CDCl₃) δ 7.12 (d, 2H), 7.2 (d, 1H), 7.6 (d, 1H), 7.82 (s, 1H), 8.26 (d, 2H).

### Step B: Preparation of 4-(4-aminophenoxy)-3-chlorobenzonitrile

To a solution of 3-chloro 4-(4-nitrophenoxy)benzonitrile (3 g, 10.94 mmol) (i.e. the product of Example 2, Step A) in ethanol (30 mL) and water (6 mL) was added iron powder (3.0 g, 54.74 mmol) and ammonium chloride (292 mg, 5.47 mmol). The mixture was heated at 80° C. for 4 hours. The reaction mixture was filtered through celite and concentrated under reduced pressure to give 2.5 g of the title compound as a solid.

¹H NMR (CDCl₃) δ3.7 (bs, 2H), 6.7-6.9 (m, 5H), 7.4 (d, 1H), 7.7 (s, 1H).

### Step C: Preparation of 4-(2-chloro-4-cyanophenoxy)benzenesulfonyl chloride

To a solution of 4-(4-aminophenoxy)-3-chlorobenzonitrile (2.0 g, 8.19 mmol) (i.e. the product of Example 2, Step B) in concentrated hydrochloric acid (12 mL) at −10° C. was added dropwise a solution of sodium nitrite (0.588 g, 8.52 mmol) in water (7 mL). The reaction mixture was stirred for 1 hr. The mixture was then added to a solution of thionyl chloride (3.89 g, 32.78 mmol) and copper (I) chloride (0.040 g, 0.409 mmol) in water (22 mL) and the mixture was stirred at room temperature for 16 hr. The mixture was treated with water and extracted with ethyl acetate. The organic phase was washed with water and saturated aqueous NaCl solution and then dried over sodium sulfate. The solvent was concentrated under reduced pressure at 40° C. and chromatographed on silica gel (10% EtOAc/hexane as eluent) to provide 500 mg of the title compound as a solid.

¹H NMR (CDCl₃) δ 7.10 (d, 2H), 7.2 (d, 1H), 7.6 (m, 1H), 7.82 (s, 1H) 8.1 (d, 2H).

### Step D: Preparation of 4-(2-chloro-4-cyanophenoxy)-N-(quinolinylmethyl)-benzenesulfonamide

To a solution of 4-quinolinemethanamine (0.142 g, 0.733 mmol) (i.e. the product of Example 1, Step B) in dichloromethane (10 mL) was added triethylamine (0.14 ml, 1.834 mmol), followed by 4-(2-chloro-4-cyanophenoxy)benzenesulfonyl chloride (0.3 g, 0.917 mmol) (i.e. the product of Example 2, Step C). The reaction mixture was stirred at room temperature for 16 hr. The solvent was concentrated and crude product was chromatographed on silica gel with 75% EtOAc/hexanes as eluent to afford the title compound, a compound of the invention, as solid (100 mg), m.p. 83-86° C.

¹H NMR (CDCl₃) δ 4.7 (d, 2H), 4.9 (m, 1H), 7.0 (m, 3H), 7.3 (d, 1H), 7.6 (m, 2H), 7.7 (m, 1H), 7.9 (m, 4H), 8.1 (d, 1H), 8.8 (d, 1H).

### SYNTHESIS EXAMPLE 3

### Preparation of methyl 2-[4-[[(4-quinolinylmethyl)amino]sulfonyl]phenoxy]benzoate (Compound Number 16)

### Step A: Preparation of methyl 2-(4-nitrophenoxy)benzoate

To a solution of 2-hydroxy methylbenzoate (2.1 g, 14.1 mmol) in acetone (40 mL) was added 4-fluoronitrobenzene (2 g, 14.1 mmol) and cesium carbonate (14 g, 42.5 mmol). The reaction mixture was stirred at room temperature for 48 hr. The mixture was then filtered, the filtrate was concentrated under reduced pressure and the resultant residue chromatographed on silica gel (10% EtOAc/hexanes as eluent) to afford 1.5 g of title compound as solid.

¹H NMR (CDCl₃) δ 3.80 (s, 3H), 6.96 (dd, 2H), 7.16 (d, 1H), 7.40 (m, 1H), 7.60 (m, 1H), 8.0 (d, 1H) 8.2 (d, 2H).

### Step B: Preparation of methyl 2-(4-aminophenoxy)benzoate

To a solution of methyl 2-(4-nitrophenoxy)benzoate (1.7 g, 6.2 mmol) (i.e. the product of Example 3, Step A) in ethanol (20 mL) and water (6 mL) was added iron powder (1.65 g, 31.1 mmol) and ammonium chloride (174 mg, 3.1 mmol). The reaction mixture was heated at 80° C. for 4 hr. The reaction mixture was then filtered through celite and concentrated under reduced pressure to give 1.2 g of the title compound as solid.

¹H NMR (CDCl₃) δ 3.6 (b, 2H), 3.90 (s, 3H), 6.70 (d, 2H), 6.86 (m, 3H), 7.1 (m, 1H), 7.4 m, 1H), 7.82 (dd, 1H).

### Step C: Preparation of methyl 2-[4-(chlorosulfonyl)phenoxy]benzoate

To a solution of methyl 2-(4-aminophenoxy)benzoate (1.2 g, 4.9 mmol) (i.e. the product of Example 3, Step B) in concentrated hydrochloric acid (9 mL) at −10° C., was added dropwise a solution of sodium nitrite (0.354 g, 5.13 mmol) in water (5 mL). The reaction mixture was stirred for 1 hr at −10° C. The reaction mixture was then added to a solution of thionyl chloride (2.3 g, 19.7 mmol) and copper (1) chloride (0.024 g, 0.24 mmol) in water (16 mL). The mixture was stirred at room temperature for 16 hr. The reaction was treated with water and extracted with ethyl acetate. The organic phase was separated, washed with water, saturated aqueous NaCl solution and then dried over sodium sulfate. The solvent was concentrated under reduced pressure at 40° C. and the residue chromatographed on silica gel with 10% EtOAc/hexanes as eluent to afford 170 mg of the title compound as a solid.

¹H NMR (CDCl₃) δ3.8 (s, 3H), 7.00 (d, 2H), 7.2 (d, 1H), 7.4 (m, 1H) 7.6 (m, 1H), 8.00 (d, 2H) 8.1 (d, 1H).

### Step D: Preparation of methyl 2-[4-[[(4-quinolinylmethyl)amino]sulfonyl]phenoxy]-benzoate

To a solution of 4-quinolinemethanamine (0.100 g, 0.51 mmol) (i.e. the product of Example 1, Step B) in dichloromethane (8 mL) was added triethylamine (0.1 ml, 0.77 mmol) and methyl 2-[4-(chlorosulfonyl)phenoxy]benzoate (0.167 g, 0.51 mmol) (i.e. the product of Example 3, Step C). The reaction mixture was stirred at room temperature for 16 hr. The solvent was concentrated and the residue chromatographed on silica gel with 75% EtOAc/hexanes as the eluent to afford the title compound, a compound of the invention, as a solid (130 mg); m.p. 163-166° C.

¹H NMR (CDCl₃) δ 3.80 (s, 3H), 4.60 (d, 2H), 4.80 (m, 1H), 6.90 (d, 2H), 7.12 (d, 1H), 7.36 (m, 2H), 7.60 (m, 2H), 7.74 (m, 1H), 7.82 (d, 2H), 7.92 (d, 1H), 8.00 (d, 1H) 8.14 (d, 1H), 8.84 (d, 1H).

### SYNTHESIS EXAMPLE 4

### Preparation of 4-(3-chloro-2-cyanophenoxy)-N-(4-quinolinylmethyl)benzenesulfonamide (Compound Number 2)

To 4-quinolinemethanamine (80 mgs, 0.51 mmol) (i.e. the product of Example 1, Step B) and diisopropylamine (195 μL, 1.11 mmol) in 20 mL tetrahydrofuran was added 4-(3-chloro-2-cyanophenoxy)benzenesulfonyl chloride (332 mg, 1.25 mmol). The reaction mixture was stirred at room temperature overnight. To this mixture 1N hydrochloric acid (2 mL) was added and the reaction was stirred to precipitate a solid. Excess water was then added and the slurry stirred for 5 min. The mixture was filtered and washed with water and diethyl ether to afford the title compound, a compound of the invention, as a solid (76 mg).

¹H NMR (DMSO) δ 9.13 (d, 1H), 8.70 (t, 1H), 8.35 (d, 1H), 8.25 (d, 1H), 8.02 (t, 1H), 7.91 (d, 1H), 7.84 (m, 1H), 7.77 (d, 1H), 7.60 (d, 1H), 7.34 (m, 2H), 7.13 (d, 1H), 4.75 (d, 2H).

By the procedures described herein together with methods known in the art, the following compounds of Tables 1 to 20 can be prepared. The following abbreviations are used in Tables 1 to 20 which follow: Me means methyl, Et means ethyl, Pr means propyl, Bu means butyl, n means normal, i means iso, OMe means methoxy, SMe means methylthio, —CN means cyano, NO₂ means nitro and Ph means phenyl.

Fragments Q¹-1 through Q¹-14 shown below are referred to in Tables 1 through 17.

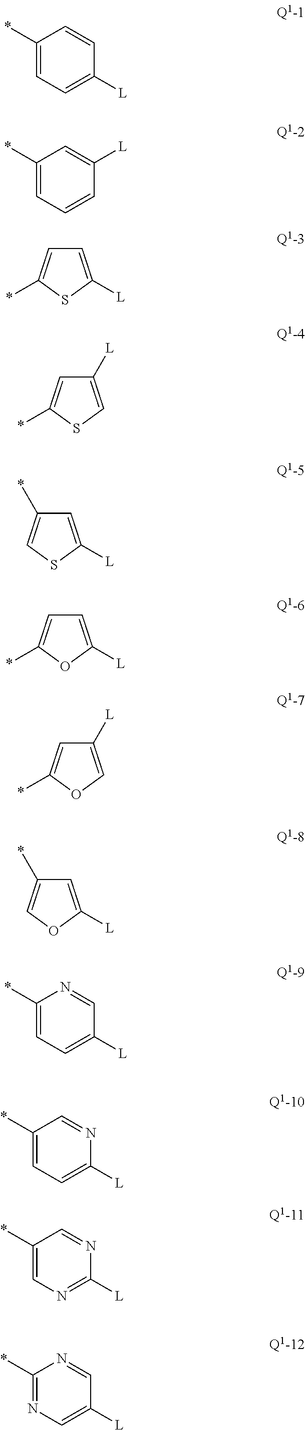

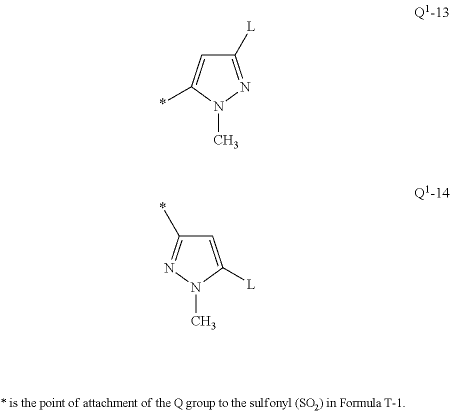

Tables 1-17 pertain to the structure of Formula T-1 shown below. (R³)ₘ represents one or a combination of substituents and no (R³)ₘ substituents is represented by a dash “-”.

TABLE 1
T-1

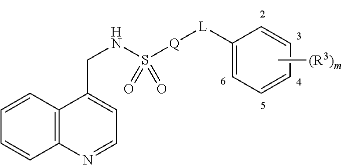

L is CH₂

| (R³)ₘ | Q |
|---|---|
| — | Q¹-1 |
| 2-F | Q¹-1 |
| 3-F | Q¹-1 |
| 4-F | Q¹-1 |
| 2-Cl | Q¹-1 |
| 3-Cl | Q¹-1 |
| 4-Cl | Q¹-1 |
| 2-Br | Q¹-1 |
| 3-Br | Q¹-1 |
| 4-Br | Q¹-1 |
| 2-I | Q¹-1 |
| 3-I | Q¹-1 |
| 4-I | Q¹-1 |
| 2-Me | Q¹-1 |
| 3-Me | Q¹-1 |
| 4-Me | Q¹-1 |
| 2-OMe | Q¹-1 |
| 3-OMe | Q¹-1 |
| 4-OMe | Q¹-1 |
| 2-CF₃ | Q¹-1 |
| 3-CF₃ | Q¹-1 |
| 4-CF₃ | Q¹-1 |
| 2-OCF₃ | Q¹-1 |
| 3-OCF₃ | Q¹-1 |
| 4-OCF₃ | Q¹-1 |
| 2-NO₂ | Q¹-1 |
| 3-NO₂ | Q¹-1 |
| 4-NO₂ | Q¹-1 |
| 2-CO₂Me | Q¹-1 |
| 3-CO₂Me | Q¹-1 |
| 4-CO₂Me | Q¹-1 |
| 2-SMe | Q¹-1 |
| 3-SMe | Q¹-1 |
| 4-SMe | Q¹-1 |
| 2-SO₂Me | Q¹-1 |
| 3-SO₂Me | Q¹-1 |
| 4-SO₂Me | Q¹-1 |
| 2-Ph | Q¹-1 |
| 3-Ph | Q¹-1 |
| 4-Ph | Q¹-1 |
| 2-OPh | Q¹-1 |
| 3-OPh | Q¹-1 |
| 4-OPh | Q¹-1 |
| — | Q¹-4 |
| 2-F | Q¹-4 |
| 3-F | Q¹-4 |
| 4-F | Q¹-4 |
| 2-Cl | Q¹-4 |
| 3-Cl | Q¹-4 |
| 4-Cl | Q¹-4 |
| 2-Br | Q¹-4 |
| 3-Br | Q¹-4 |
| 4-Br | Q¹-4 |
| 2-I | Q¹-4 |
| 3-I | Q¹-4 |
| 4-I | Q¹-4 |
| 2-Me | Q¹-4 |
| 3-Me | Q¹-4 |
| 4-Me | Q¹-4 |
| 2-OMe | Q¹-4 |
| 3-OMe | Q¹-4 |
| 4-OMe | Q¹-4 |
| 2-CF₃ | Q¹-4 |
| 3-CF₃ | Q¹-4 |
| 4-CF₃ | Q¹-4 |
| 2-OCF₃ | Q¹-4 |
| 3-OCF₃ | Q¹-4 |
| 4-OCF₃ | Q¹-4 |
| 2-NO₂ | Q¹-4 |
| 3-NO₂ | Q¹-4 |
| 4-NO₂ | Q¹-4 |
| 2-CO₂Me | Q¹-4 |
| 3-CO₂Me | Q¹-4 |
| 4-CO₂Me | Q¹-4 |
| 2-SMe | Q¹-4 |
| 3-SMe | Q¹-4 |
| 4-SMe | Q¹-4 |
| 2-SO₂Me | Q¹-4 |
| 3-SO₂Me | Q¹-4 |
| 4-SO₂Me | Q¹-4 |
| 2-Ph | Q¹-4 |
| 3-Ph | Q¹-4 |
| 4-Ph | Q¹-4 |
| 2-OPh | Q¹-4 |
| 3-OPh | Q¹-4 |
| 4-OPh | Q¹-4 |
| — | Q¹-7 |
| 2-F | Q¹-7 |
| 3-F | Q¹-7 |
| 4-F | Q¹-7 |
| 2-Cl | Q¹-7 |
| 3-Cl | Q¹-7 |
| 4-Cl | Q¹-7 |
| 2-Br | Q¹-7 |
| 3-Br | Q¹-7 |
| 4-Br | Q¹-7 |
| 2-I | Q¹-7 |
| 3-I | Q¹-7 |
| 4-I | Q¹-7 |
| 2-Me | Q¹-7 |
| 3-Me | Q¹-7 |
| 4-Me | Q¹-7 |
| 2-OMe | Q¹-7 |
| 3-OMe | Q¹-7 |
| 4-OMe | Q¹-7 |
| 2-CF₃ | Q¹-7 |
| 3-CF₃ | Q¹-7 |
| 4-CF₃ | Q¹-7 |
| 2-OCF₃ | Q¹-7 |
| 3-OCF₃ | Q¹-7 |
| 4-OCF₃ | Q¹-7 |
| 2-NO₂ | Q¹-7 |
| 3-NO₂ | Q¹-7 |
| 4-NO₂ | Q¹-7 |
| 2-CO₂Me | Q¹-7 |
| 3-CO₂Me | Q¹-7 |
| 4-CO₂Me | Q¹-7 |
| 2-SMe | Q¹-7 |
| 3-SMe | Q¹-7 |
| 4-SMe | Q¹-7 |
| 2-SO₂Me | Q¹-7 |
| 3-SO₂Me | Q¹-7 |
| 4-SO₂Me | Q¹-7 |
| 2-Ph | Q¹-7 |
| 3-Ph | Q¹-7 |
| 4-Ph | Q¹-7 |
| 2-OPh | Q¹-7 |
| 3-OPh | Q¹-7 |
| 4-OPh | Q¹-7 |
| — | Q¹-10 |
| 2-F | Q¹-10 |
| 3-F | Q¹-10 |
| 4-F | Q¹-10 |
| 2-Cl | Q¹-10 |
| 3-Cl | Q¹-10 |
| 4-Cl | Q¹-10 |
| 2-Br | Q¹-10 |
| 3-Br | Q¹-10 |
| 4-Br | Q¹-10 |
| 2-I | Q¹-10 |
| 3-I | Q¹-10 |
| 4-I | Q¹-10 |
| 2-Me | Q¹-10 |
| 3-Me | Q¹-10 |
| 4-Me | Q¹-10 |
| 2-OMe | Q¹-10 |
| 3-OMe | Q¹-10 |
| 4-OMe | Q¹-10 |
| 2-CF₃ | Q¹-10 |
| 3-CF₃ | Q¹-10 |
| 4-CF₃ | Q¹-10 |
| 2-OCF₃ | Q¹-10 |
| 3-OCF₃ | Q¹-10 |
| 4-OCF₃ | Q¹-10 |
| 2-NO₂ | Q¹-10 |
| 3-NO₂ | Q¹-10 |
| 4-NO₂ | Q¹-10 |
| 2-CO₂Me | Q¹-10 |
| 3-CO₂Me | Q¹-10 |
| 4-CO₂Me | Q¹-10 |
| 2-SMe | Q¹-10 |
| 3-SMe | Q¹-10 |
| 4-SMe | Q¹-10 |
| 2-SO₂Me | Q¹-10 |
| 3-SO₂Me | Q¹-10 |
| 4-SO₂Me | Q¹-10 |
| 2-Ph | Q¹-10 |
| 3-Ph | Q¹-10 |
| 4-Ph | Q¹-10 |
| 2-OPh | Q¹-10 |
| 3-OPh | Q¹-10 |
| 4-OPh | Q¹-10 |
| — | Q¹-13 |
| 2-F | Q¹-13 |
| 3-F | Q¹-13 |
| 4-F | Q¹-13 |
| 2-Cl | Q¹-13 |
| 3-Cl | Q¹-13 |
| 4-Cl | Q¹-13 |
| 2-Br | Q¹-13 |
| 3-Br | Q¹-13 |
| 4-Br | Q¹-13 |
| 2-I | Q¹-13 |
| 3-I | Q¹-13 |
| 4-I | Q¹-13 |
| 2-Me | Q¹-13 |
| 3-Me | Q¹-13 |
| 4-Me | Q¹-13 |
| 2-OMe | Q¹-13 |
| 3-OMe | Q¹-13 |
| 4-OMe | Q¹-13 |
| 2-CF₃ | Q¹-13 |
| 3-CF₃ | Q¹-13 |
| 4-CF₃ | Q¹-13 |
| 2-OCF₃ | Q¹-13 |
| 3-OCF₃ | Q¹-13 |
| 4-OCF₃ | Q¹-13 |
| 2-NO₂ | Q¹-13 |
| 3-NO₂ | Q¹-13 |
| 4-NO₂ | Q¹-13 |
| 2-CO₂Me | Q¹-13 |
| — | Q¹-2 |
| 2-F | Q¹-2 |
| 3-F | Q¹-2 |
| 4-F | Q¹-2 |
| 2-Cl | Q¹-2 |
| 3-Cl | Q¹-2 |
| 4-Cl | Q¹-2 |
| 2-Br | Q¹-2 |
| 3-Br | Q¹-2 |
| 4-Br | Q¹-2 |
| 2-I | Q¹-2 |
| 3-I | Q¹-2 |
| 4-I | Q¹-2 |
| 2-Me | Q¹-2 |
| 3-Me | Q¹-2 |
| 4-Me | Q¹-2 |
| 2-OMe | Q¹-2 |
| 3-OMe | Q¹-2 |
| 4-OMe | Q¹-2 |
| 2-CF₃ | Q¹-2 |
| 3-CF₃ | Q¹-2 |
| 4-CF₃ | Q¹-2 |
| 2-OCF₃ | Q¹-2 |
| 3-OCF₃ | Q¹-2 |
| 4-OCF₃ | Q¹-2 |
| 2-NO₂ | Q¹-2 |
| 3-NO₂ | Q¹-2 |
| 4-NO₂ | Q¹-2 |
| 2-CO₂Me | Q¹-2 |
| 3-CO₂Me | Q¹-2 |
| 4-CO₂Me | Q¹-2 |
| 2-SMe | Q¹-2 |
| 3-SMe | Q¹-2 |
| 4-SMe | Q¹-2 |
| 2-SO₂Me | Q¹-2 |
| 3-SO₂Me | Q¹-2 |
| 4-SO₂Me | Q¹-2 |
| 2-Ph | Q¹-2 |
| 3-Ph | Q¹-2 |
| 4-Ph | Q¹-2 |
| 2-OPh | Q¹-2 |
| 3-OPh | Q¹-2 |
| 4-OPh | Q¹-2 |
| — | Q¹-5 |
| 2-F | Q¹-5 |
| 3-F | Q¹-5 |
| 4-F | Q¹-5 |
| 2-Cl | Q¹-5 |
| 3-Cl | Q¹-5 |
| 4-Cl | Q¹-5 |
| 2-Br | Q¹-5 |
| 3-Br | Q¹-5 |
| 4-Br | Q¹-5 |
| 2-I | Q¹-5 |
| 3-I | Q¹-5 |
| 4-I | Q¹-5 |
| 2-Me | Q¹-5 |
| 3-Me | Q¹-5 |
| 4-Me | Q¹-5 |
| 2-OMe | Q¹-5 |
| 3-OMe | Q¹-5 |
| 4-OMe | Q¹-5 |
| 2-CF₃ | Q¹-5 |
| 3-CF₃ | Q¹-5 |
| 4-CF₃ | Q¹-5 |
| 2-OCF₃ | Q¹-5 |
| 3-OCF₃ | Q¹-5 |
| 4-OCF₃ | Q¹-5 |
| 2-NO₂ | Q¹-5 |
| 3-NO₂ | Q¹-5 |
| 4-NO₂ | Q¹-5 |
| 2-CO₂Me | Q¹-5 |
| 3-CO₂Me | Q¹-5 |
| 4-CO₂Me | Q¹-5 |
| 2-SMe | Q¹-5 |
| 3-SMe | Q¹-5 |
| 4-SMe | Q¹-5 |
| 2-SO₂Me | Q¹-5 |
| 3-SO₂Me | Q¹-5 |
| 4-SO₂Me | Q¹-5 |
| 2-Ph | Q¹-5 |
| 3-Ph | Q¹-5 |
| 4-Ph | Q¹-5 |
| 2-OPh | Q¹-5 |
| 3-OPh | Q¹-5 |
| 4-OPh | Q¹-5 |
| — | Q¹-8 |
| 2-F | Q¹-8 |
| 3-F | Q¹-8 |
| 4-F | Q¹-8 |
| 2-Cl | Q¹-8 |
| 3-Cl | Q¹-8 |
| 4-Cl | Q¹-8 |
| 2-Br | Q¹-8 |
| 3-Br | Q¹-8 |
| 4-Br | Q¹-8 |
| 2-I | Q¹-8 |
| 3-I | Q¹-8 |
| 4-I | Q¹-8 |
| 2-Me | Q¹-8 |
| 3-Me | Q¹-8 |
| 4-Me | Q¹-8 |
| 2-OMe | Q¹-8 |
| 3-OMe | Q¹-8 |
| 4-OMe | Q¹-8 |
| 2-CF₃ | Q¹-8 |
| 3-CF₃ | Q¹-8 |
| 4-CF₃ | Q¹-8 |
| 2-OCF₃ | Q¹-8 |
| 3-OCF₃ | Q¹-8 |
| 4-OCF₃ | Q¹-8 |
| 2-NO₂ | Q¹-8 |
| 3-NO₂ | Q¹-8 |
| 4-NO₂ | Q¹-8 |
| 2-CO₂Me | Q¹-8 |
| 3-CO₂Me | Q¹-8 |
| 4-CO₂Me | Q¹-8 |
| 2-SMe | Q¹-8 |
| 3-SMe | Q¹-8 |
| 4-SMe | Q¹-8 |
| 2-SO₂Me | Q¹-8 |
| 3-SO₂Me | Q¹-8 |
| 4-SO₂Me | Q¹-8 |
| 2-Ph | Q¹-8 |
| 3-Ph | Q¹-8 |
| 4-Ph | Q¹-8 |
| 2-OPh | Q¹-8 |
| 3-OPh | Q¹-8 |
| 4-OPh | Q¹-8 |
| — | Q¹-11 |
| 2-F | Q¹-11 |
| 3-F | Q¹-11 |
| 4-F | Q¹-11 |
| 2-Cl | Q¹-11 |
| 3-Cl | Q¹-11 |
| 4-Cl | Q¹-11 |
| 2-Br | Q¹-11 |
| 3-Br | Q¹-11 |
| 4-Br | Q¹-11 |
| 2-I | Q¹-11 |
| 3-I | Q¹-11 |
| 4-I | Q¹-11 |
| 2-Me | Q¹-11 |
| 3-Me | Q¹-11 |
| 4-Me | Q¹-11 |
| 2-OMe | Q¹-11 |
| 3-OMe | Q¹-11 |
| 4-OMe | Q¹-11 |
| 2-CF₃ | Q¹-11 |
| 3-CF₃ | Q¹-11 |
| 4-CF₃ | Q¹-11 |
| 2-OCF₃ | Q¹-11 |
| 3-OCF₃ | Q¹-11 |
| 4-OCF₃ | Q¹-11 |
| 2-NO₂ | Q¹-11 |
| 3-NO₂ | Q¹-11 |
| 4-NO₂ | Q¹-11 |
| 2-CO₂Me | Q¹-11 |
| 3-CO₂Me | Q¹-11 |
| 4-CO₂Me | Q¹-11 |
| 2-SMe | Q¹-11 |
| 3-SMe | Q¹-11 |
| 4-SMe | Q¹-11 |
| 2-SO₂Me | Q¹-11 |
| 3-SO₂Me | Q¹-11 |
| 4-SO₂Me | Q¹-11 |
| 2-Ph | Q¹-11 |
| 3-Ph | Q¹-11 |
| 4-Ph | Q¹-11 |
| 2-OPh | Q¹-11 |
| 3-OPh | Q¹-11 |
| 4-OPh | Q¹-11 |
| 3-CO₂Me | Q¹-13 |
| 4-CO₂Me | Q¹-13 |
| 2-SMe | Q¹-13 |
| 3-SMe | Q¹-13 |
| 4-SMe | Q¹-13 |
| 2-SO₂Me | Q¹-13 |
| 3-SO₂Me | Q¹-13 |
| 4-SO₂Me | Q¹-13 |
| 2-Ph | Q¹-13 |
| 3-Ph | Q¹-13 |
| 4-Ph | Q¹-13 |
| 2-OPh | Q¹-13 |
| 3-OPh | Q¹-13 |
| 4-OPh | Q¹-13 |
| — | Q¹-14 |
| 2-F | Q¹-14 |
| 3-F | Q¹-14 |
| 4-F | Q¹-14 |
| 2-Cl | Q¹-14 |
| 3-Cl | Q¹-14 |
| 4-Cl | Q¹-14 |
| 2-Br | Q¹-14 |
| 3-Br | Q¹-14 |
| 4-Br | Q¹-14 |
| 2-I | Q¹-14 |
| 3-I | Q¹-14 |
| 4-I | Q¹-14 |
| 2-Me | Q¹-14 |
| 3-Me | Q¹-14 |
| — | Q¹-3 |
| 2-F | Q¹-3 |
| 3-F | Q¹-3 |
| 4-F | Q¹-3 |
| 2-Cl | Q¹-3 |
| 3-Cl | Q¹-3 |
| 4-Cl | Q¹-3 |
| 2-Br | Q¹-3 |
| 3-Br | Q¹-3 |
| 4-Br | Q¹-3 |
| 2-I | Q¹-3 |
| 3-I | Q¹-3 |
| 4-I | Q¹-3 |
| 2-Me | Q¹-3 |
| 3-Me | Q¹-3 |
| 4-Me | Q¹-3 |
| 2-OMe | Q¹-3 |
| 3-OMe | Q¹-3 |
| 4-OMe | Q¹-3 |
| 2-CF₃ | Q¹-3 |
| 3-CF₃ | Q¹-3 |
| 4-CF₃ | Q¹-3 |
| 2-OCF₃ | Q¹-3 |
| 3-OCF₃ | Q¹-3 |
| 4-OCF₃ | Q¹-3 |
| 2-NO₂ | Q¹-3 |
| 3-NO₂ | Q¹-3 |
| 4-NO₂ | Q¹-3 |
| 2-CO₂Me | Q¹-3 |
| 3-CO₂Me | Q¹-3 |
| 4-CO₂Me | Q¹-3 |
| 2-SMe | Q¹-3 |
| 3-SMe | Q¹-3 |
| 4-SMe | Q¹-3 |
| 2-SO₂Me | Q¹-3 |
| 3-SO₂Me | Q¹-3 |
| 4-SO₂Me | Q¹-3 |
| 2-Ph | Q¹-3 |
| 3-Ph | Q¹-3 |
| 4-Ph | Q¹-3 |
| 2-OPh | Q¹-3 |
| 3-OPh | Q¹-3 |
| 4-OPh | Q¹-3 |
| — | Q¹-6 |
| 2-F | Q¹-6 |
| 3-F | Q¹-6 |
| 4-F | Q¹-6 |
| 2-Cl | Q¹-6 |
| 3-Cl | Q¹-6 |
| 4-Cl | Q¹-6 |
| 2-Br | Q¹-6 |
| 3-Br | Q¹-6 |
| 4-Br | Q¹-6 |
| 2-I | Q¹-6 |
| 3-I | Q¹-6 |
| 4-I | Q¹-6 |
| 2-Me | Q¹-6 |
| 3-Me | Q¹-6 |
| 4-Me | Q¹-6 |
| 2-OMe | Q¹-6 |
| 3-OMe | Q¹-6 |
| 4-OMe | Q¹-6 |
| 2-CF₃ | Q¹-6 |
| 3-CF₃ | Q¹-6 |
| 4-CF₃ | Q¹-6 |
| 2-OCF₃ | Q¹-6 |
| 3-OCF₃ | Q¹-6 |
| 4-OCF₃ | Q¹-6 |
| 2-NO₂ | Q¹-6 |
| 3-NO₂ | Q¹-6 |
| 4-NO₂ | Q¹-6 |
| 2-CO₂Me | Q¹-6 |
| 3-CO₂Me | Q¹-6 |
| 4-CO₂Me | Q¹-6 |
| 2-SMe | Q¹-6 |
| 3-SMe | Q¹-6 |
| 4-SMe | Q¹-6 |
| 2-SO₂Me | Q¹-6 |
| 3-SO₂Me | Q¹-6 |
| 4-SO₂Me | Q¹-6 |
| 2-Ph | Q¹-6 |
| 3-Ph | Q¹-6 |
| 4-Ph | Q¹-6 |
| 2-OPh | Q¹-6 |
| 3-OPh | Q¹-6 |
| 4-OPh | Q¹-6 |
| — | Q¹-9 |
| 2-F | Q¹-9 |
| 3-F | Q¹-9 |
| 4-F | Q¹-9 |
| 2-Cl | Q¹-9 |
| 3-Cl | Q¹-9 |
| 4-Cl | Q¹-9 |
| 2-Br | Q¹-9 |
| 3-Br | Q¹-9 |
| 4-Br | Q¹-9 |
| 2-I | Q¹-9 |
| 3-I | Q¹-9 |
| 4-I | Q¹-9 |
| 2-Me | Q¹-9 |
| 3-Me | Q¹-9 |
| 4-Me | Q¹-9 |
| 2-OMe | Q¹-9 |
| 3-OMe | Q¹-9 |
| 4-OMe | Q¹-9 |
| 2-CF₃ | Q¹-9 |
| 3-CF₃ | Q¹-9 |
| 4-CF₃ | Q¹-9 |
| 2-OCF₃ | Q¹-9 |
| 3-OCF₃ | Q¹-9 |
| 4-OCF₃ | Q¹-9 |
| 2-NO₂ | Q¹-9 |
| 3-NO₂ | Q¹-9 |
| 4-NO₂ | Q¹-9 |
| 2-CO₂Me | Q¹-9 |
| 3-CO₂Me | Q¹-9 |
| 4-CO₂Me | Q¹-9 |
| 2-SMe | Q¹-9 |
| 3-SMe | Q¹-9 |
| 4-SMe | Q¹-9 |
| 2-SO₂Me | Q¹-9 |
| 3-SO₂Me | Q¹-9 |
| 4-SO₂Me | Q¹-9 |
| 2-Ph | Q¹-9 |
| 3-Ph | Q¹-9 |
| 4-Ph | Q¹-9 |
| 2-OPh | Q¹-9 |
| 3-OPh | Q¹-9 |
| 4-OPh | Q¹-9 |
| — | Q¹-12 |
| 2-F | Q¹-12 |
| 3-F | Q¹-12 |
| 4-F | Q¹-12 |
| 2-Cl | Q¹-12 |
| 3-Cl | Q¹-12 |
| 4-Cl | Q¹-12 |
| 2-Br | Q¹-12 |
| 3-Br | Q¹-12 |
| 4-Br | Q¹-12 |
| 2-I | Q¹-12 |
| 3-I | Q¹-12 |
| 4-I | Q¹-12 |
| 2-Me | Q¹-12 |
| 3-Me | Q¹-12 |
| 4-Me | Q¹-12 |
| 2-OMe | Q¹-12 |
| 3-OMe | Q¹-12 |
| 4-OMe | Q¹-12 |
| 2-CF₃ | Q¹-12 |
| 3-CF₃ | Q¹-12 |
| 4-CF₃ | Q¹-12 |
| 2-OCF₃ | Q¹-12 |
| 3-OCF₃ | Q¹-12 |
| 4-OCF₃ | Q¹-12 |
| 2-NO₂ | Q¹-12 |
| 3-NO₂ | Q¹-12 |
| 4-NO₂ | Q¹-12 |
| 2-CO₂Me | Q¹-12 |
| 3-CO₂Me | Q¹-12 |
| 4-CO₂Me | Q¹-12 |
| 2-SMe | Q¹-12 |
| 3-SMe | Q¹-12 |
| 4-SMe | Q¹-12 |
| 2-SO₂Me | Q¹-12 |
| 3-SO₂Me | Q¹-12 |
| 4-SO₂Me | Q¹-12 |
| 2-Ph | Q¹-12 |
| 3-Ph | Q¹-12 |
| 4-Ph | Q¹-12 |
| 2-OPh | Q¹-12 |
| 3-OPh | Q¹-12 |
| 4-OPh | Q¹-12 |
| 4-Me | Q¹-14 |
| 2-OMe | Q¹-14 |
| 3-OMe | Q¹-14 |
| 4-OMe | Q¹-14 |
| 2-CF₃ | Q¹-14 |
| 3-CF₃ | Q¹-14 |
| 4-CF₃ | Q¹-14 |
| 2-OCF₃ | Q¹-14 |
| 3-OCF₃ | Q¹-14 |
| 4-OCF₃ | Q¹-14 |
| 2-NO₂ | Q¹-14 |
| 3-NO₂ | Q¹-14 |
| 4-NO₂ | Q¹-14 |
| 2-CO₂Me | Q¹-14 |
| 3-CO₂Me | Q¹-14 |
| 4-CO₂Me | Q¹-14 |
| 2-SMe | Q¹-14 |
| 3-SMe | Q¹-14 |
| 4-SMe | Q¹-14 |
| 2-SO₂Me | Q¹-14 |
| 3-SO₂Me | Q¹-14 |
| 4-SO₂Me | Q¹-14 |
| 2-Ph | Q¹-14 |
| 3-Ph | Q¹-14 |
| 4-Ph | Q¹-14 |
| 2-OPh | Q¹-14 |
| 3-OPh | Q¹-14 |
| 4-OPh | Q¹-14 |

The present disclosure also includes Table 2 through 17, each of which is constructed the same as Table 1 above except that the table heading in Table 1 (i.e. “L is CH₂” is replaced with the respective table headings shown below. For example, in Table 2 the table heading is “L is CHF” and R³ and Q are as defined in Table 1 above. Thus, the first entry in Table 2 specifically discloses a compound of Formula 1 wherein L is CHF, Q is Q-1 and there is no (R³)ₘ substituent.

| Table | Table Headings |
|---|---|
| 2 | L is CHF |
| 3 | L is CF₂ |
| 4 | L is CH₂CH₂ |
| 5 | L is CH═CH |
| 6 | L is CH≡CH |
| 7 | L is OCH₂ |
| 8 | L is CO |
| 9 | L is S |
| 10 | L is SO |
| 11 | L is SO₂ |
| 12 | L is NH |
| 13 | L is NCH₃ |
| 14 | L is CONH |
| 15 | L is NHCO |
| 16 | L is SO₂NH |
| 17 | L is NHSO₂ |

TABLE 18

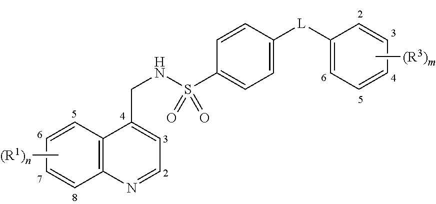

(R¹)ₙ or (R³)ₘ represent one or a combination of substituents and no
(R¹)ₙ or (R³)ₘ substituents is represented by a dash “—”.

| (R¹)ₙ | (R³)ₘ | L |
|---|---|---|
| 2-F | — | CH₂ |
| 3-F | — | CH₂ |
| 5-F | — | CH₂ |
| 6-F | — | CH₂ |
| 7-F | — | CH₂ |
| 8-F | — | CH₂ |
| 2-Cl | — | CH₂ |
| 3-Cl | — | CH₂ |
| 5-Cl | — | CH₂ |
| 6-Cl | — | CH₂ |
| 7-Cl | — | CH₂ |
| 8-Cl | — | CH₂ |
| 2-CF₃ | — | CH₂ |
| 3-CF₃ | — | CH₂ |
| 5-CF₃ | — | CH₂ |
| 6-CF₃ | — | CH₂ |
| 7-CF₃ | — | CH₂ |
| 8-CF₃ | — | CH₂ |
| 2-Me | — | CH₂ |
| 3-Me | — | CH₂ |
| 5-Me | — | CH₂ |
| 6-Me | — | CH₂ |
| 7-Me | — | CH₂ |
| 8-Me | — | CH₂ |
| 2-OMe | — | CH₂ |
| 2-OEt | — | CH₂ |
| 2-O-iPr | — | CH₂ |
| 2-O-iBu | — | CH₂ |
| 2-O-nBu | — | CH₂ |
| 5,7-diCl | — | CH₂ |
| 6,7-diCl | — | CH₂ |
| 5,8-diCl | — | CH₂ |
| 7,8-diCl | — | CH₂ |
| 5,7-diCl | — | CH₂ |
| 6,8-diF | — | CH₂ |
| 7,8-diF | — | CH₂ |
| 2-F | — | CH₂ |
| 3-F | 4-Cl | CH₂ |
| 5-F | 4-Cl | CH₂ |
| 6-F | 4-Cl | CH₂ |
| 7-F | 4-Cl | CH₂ |
| 8-F | 4-Cl | CH₂ |
| 2-Cl | 4-Cl | CH₂ |
| 3-Cl | 4-Cl | CH₂ |
| 5-Cl | 4-Cl | CH₂ |
| 6-Cl | 4-Cl | CH₂ |
| 7-Cl | 4-Cl | CH₂ |
| 8-Cl | 4-Cl | CH₂ |
| 2-CF₃ | 4-Cl | CH₂ |
| 3-CF₃ | 4-Cl | CH₂ |
| 5-CF₃ | 4-Cl | CH₂ |
| 6-CF₃ | 4-Cl | CH₂ |
| 7-CF₃ | 4-Cl | CH₂ |
| 8-CF₃ | 4-Cl | CH₂ |
| 2-Me | 4-Cl | CH₂ |
| 3-Me | 4-Cl | CH₂ |
| 5-Me | 4-Cl | CH₂ |
| 6-Me | 4-Cl | CH₂ |
| 7-Me | 4-Cl | CH₂ |
| 8-Me | 4-Cl | CH₂ |
| 2-OMe | 4-Cl | CH₂ |
| 2-OEt | 4-Cl | CH₂ |
| 2-O-iPr | 4-Cl | CH₂ |
| 2-O-iBu | 4-Cl | CH₂ |
| 2-O-nBu | 4-Cl | CH₂ |
| 5,7-diCl | 4-Cl | CH₂ |
| 6,7-diCl | 4-Cl | CH₂ |
| 5,8-diCl | 4-Cl | CH₂ |
| 7,8-diCl | 4-Cl | CH₂ |
| 5,7-diCl | 4-Cl | CH₂ |
| 6,8-diF | 4-Cl | CH₂ |
| 7,8-diF | 4-Cl | CH₂ |
| 2-F | 4-F | OCH₂ |
| 3-F | 4-F | OCH₂ |
| 5-F | 4-F | OCH₂ |
| 6-F | 4-F | OCH₂ |
| 7-F | 4-F | OCH₂ |
| 8-F | 4-F | OCH₂ |
| 2-Cl | 4-F | OCH₂ |
| 3-Cl | 4-F | OCH₂ |
| 5-Cl | 4-F | OCH₂ |
| 6-Cl | 4-F | OCH₂ |
| 7-Cl | 4-F | OCH₂ |
| 8-Cl | 4-F | OCH₂ |
| 2-CF₃ | 4-F | OCH₂ |
| 3-CF₃ | 4-F | OCH₂ |
| 5-CF₃ | 4-F | OCH₂ |
| 6-CF₃ | 4-F | OCH₂ |
| 7-CF₃ | 4-F | OCH₂ |
| 8-CF₃ | 4-F | OCH₂ |
| 2-Me | 4-F | OCH₂ |
| 3-Me | 4-F | OCH₂ |
| 5-Me | 4-F | OCH₂ |
| 6-Me | 4-F | OCH₂ |
| 7-Me | 4-F | OCH₂ |
| 8-Me | 4-F | OCH₂ |
| 2-OMe | 4-F | OCH₂ |
| 2-OEt | 4-F | OCH₂ |
| 2-O-iPr | 4-F | OCH₂ |
| 2-O-iBu | 4-F | OCH₂ |
| 2-O-nBu | 4-F | OCH₂ |
| 5,7-diCl | 4-F | OCH₂ |
| 6,7-diCl | 4-F | OCH₂ |
| 5,8-diCl | 4-F | OCH₂ |
| 7,8-diCl | 4-F | OCH₂ |
| 5,7-diCl | 4-F | OCH₂ |
| 6,8-diF | 4-F | OCH₂ |
| 7,8-diF | 4-F | OCH₂ |
| 2-F | — | C≡C |
| 3-F | — | C≡C |
| 5-F | — | C≡C |
| 6-F | — | C≡C |
| 7-F | — | C≡C |
| 8-F | — | C≡C |
| 2-Cl | — | C≡C |
| 3-Cl | — | C≡C |
| 5-Cl | — | C≡C |
| 6-Cl | — | C≡C |
| 7-Cl | — | C≡C |
| 8-Cl | — | C≡C |
| 2-CF₃ | — | C≡C |
| 3-CF₃ | — | C≡C |
| 5-CF₃ | — | C≡C |
| 6-CF₃ | — | C≡C |
| 7-CF₃ | — | C≡C |
| 8-CF₃ | — | C≡C |
| 2-Me | — | C≡C |
| 3-Me | — | C≡C |
| 5-Me | — | C≡C |
| 6-Me | — | C≡C |
| 7-Me | — | C≡C |
| 8-Me | — | C≡C |
| 2-OMe | — | C≡C |
| 2-OEt | — | C≡C |
| 2-O-iPr | — | C≡C |
| 2-O-iBu | — | C≡C |
| 2-O-nBu | — | C≡C |
| 5,7-diCl | — | C≡C |
| 6,7-diCl | — | C≡C |
| 5,8-diCl | — | C≡C |
| 7,8-diCl | — | C≡C |
| 5,7-diCl | — | C≡C |
| 6,8-diF | — | C≡C |
| 7,8-diF | — | C≡C |
| 2-F | 4-Cl | C≡C |
| 3-F | 4-Cl | C≡C |
| 5-F | 4-Cl | C≡C |
| 6-F | 4-Cl | C≡C |
| 7-F | 4-Cl | C≡C |
| 8-F | 4-Cl | C≡C |
| 2-Cl | 4-Cl | C≡C |
| 3-Cl | 4-Cl | C≡C |
| 5-Cl | 4-Cl | C≡C |
| 6-Cl | 4-Cl | C≡C |
| 7-Cl | 4-Cl | C≡C |
| 8-Cl | 4-Cl | C≡C |
| 2-CF₃ | 4-Cl | C≡C |
| 3-CF₃ | 4-Cl | C≡C |
| 5-CF₃ | 4-Cl | C≡C |
| 6-CF₃ | 4-Cl | C≡C |
| 7-CF₃ | 4-Cl | C≡C |
| 8-CF₃ | 4-Cl | C≡C |
| 2-F | 4-F | CH₂ |
| 3-F | 4-F | CH₂ |
| 5-F | 4-F | CH₂ |
| 6-F | 4-F | CH₂ |
| 7-F | 4-F | CH₂ |
| 8-F | 4-F | CH₂ |
| 2-Cl | 4-F | CH₂ |
| 3-Cl | 4-F | CH₂ |
| 5-Cl | 4-F | CH₂ |
| 6-Cl | 4-F | CH₂ |
| 7-Cl | 4-F | CH₂ |
| 8-Cl | 4-F | CH₂ |
| 2-CF₃ | 4-F | CH₂ |
| 3-CF₃ | 4-F | CH₂ |
| 5-CF₃ | 4-F | CH₂ |
| 6-CF₃ | 4-F | CH₂ |
| 7-CF₃ | 4-F | CH₂ |
| 8-CF₃ | 4-F | CH₂ |
| 2-Me | 4-F | CH₂ |
| 3-Me | 4-F | CH₂ |
| 5-Me | 4-F | CH₂ |
| 6-Me | 4-F | CH₂ |
| 7-Me | 4-F | CH₂ |
| 8-Me | 4-F | CH₂ |
| 2-OMe | 4-F | CH₂ |
| 2-OEt | 4-F | CH₂ |
| 2-O-iPr | 4-F | CH₂ |
| 2-O-iBu | 4-F | CH₂ |
| 2-O-nBu | 4-F | CH₂ |
| 5,7-diCl | 4-F | CH₂ |
| 6,7-diCl | 4-F | CH₂ |
| 5,8-diCl | 4-F | CH₂ |
| 7,8-diCl | 4-F | CH₂ |
| 5,7-diCl | 4-F | CH₂ |
| 6,8-diF | 4-F | CH₂ |
| 7,8-diF | 4-F | CH₂ |
| 2-F | — | OCH₂ |
| 3-F | — | OCH₂ |
| 5-F | — | OCH₂ |
| 6-F | — | OCH₂ |
| 7-F | — | OCH₂ |
| 8-F | — | OCH₂ |
| 2-Cl | — | OCH₂ |
| 3-Cl | — | OCH₂ |
| 5-Cl | — | OCH₂ |
| 6-Cl | — | OCH₂ |
| 7-Cl | — | OCH₂ |
| 8-Cl | — | OCH₂ |
| 2-CF₃ | — | OCH₂ |
| 3-CF₃ | — | OCH₂ |
| 5-CF₃ | — | OCH₂ |
| 6-CF₃ | — | OCH₂ |
| 7-CF₃ | — | OCH₂ |
| 8-CF₃ | — | OCH₂ |
| 2-Me | — | OCH₂ |
| 3-Me | — | OCH₂ |
| 5-Me | — | OCH₂ |
| 6-Me | — | OCH₂ |
| 7-Me | — | OCH₂ |
| 8-Me | — | OCH₂ |
| 2-OMe | — | OCH₂ |
| 2-OEt | — | OCH₂ |
| 2-O-iPr | — | OCH₂ |
| 2-O-iBu | — | OCH₂ |
| 2-O-nBu | — | OCH₂ |
| 5,7-diCl | — | OCH₂ |
| 6,7-diCl | — | OCH₂ |
| 5,8-diCl | — | OCH₂ |
| 7,8-diCl | — | OCH₂ |
| 5,7-diCl | — | OCH₂ |
| 6,8-diF | — | OCH₂ |
| 7,8-diF | — | OCH₂ |
| 2-F | 4-Cl | OCH₂ |
| 3-F | 4-Cl | OCH₂ |
| 5-F | 4-Cl | OCH₂ |
| 6-F | 4-Cl | OCH₂ |
| 7-F | 4-Cl | OCH₂ |
| 8-F | 4-Cl | OCH₂ |
| 2-Cl | 4-Cl | OCH₂ |
| 3-Cl | 4-Cl | OCH₂ |
| 5-Cl | 4-Cl | OCH₂ |
| 6-Cl | 4-Cl | OCH₂ |
| 7-Cl | 4-Cl | OCH₂ |
| 8-Cl | 4-Cl | OCH₂ |
| 2-CF₃ | 4-Cl | OCH₂ |
| 3-CF₃ | 4-Cl | OCH₂ |
| 5-CF₃ | 4-Cl | OCH₂ |
| 6-CF₃ | 4-Cl | OCH₂ |
| 7-CF₃ | 4-Cl | OCH₂ |
| 8-CF₃ | 4-Cl | OCH₂ |
| 2-Me | 4-Cl | OCH₂ |
| 3-Me | 4-Cl | OCH₂ |
| 5-Me | 4-Cl | OCH₂ |
| 6-Me | 4-Cl | OCH₂ |
| 7-Me | 4-Cl | OCH₂ |
| 8-Me | 4-Cl | OCH₂ |
| 2-OMe | 4-Cl | OCH₂ |
| 2-OEt | 4-Cl | OCH₂ |
| 2-O-iPr | 4-Cl | OCH₂ |
| 2-O-iBu | 4-Cl | OCH₂ |
| 2-O-nBu | 4-Cl | OCH₂ |
| 5,7-diCl | 4-Cl | OCH₂ |
| 6,7-diCl | 4-Cl | OCH₂ |
| 5,8-diCl | 4-Cl | OCH₂ |
| 7,8-diCl | 4-Cl | OCH₂ |
| 5,7-diCl | 4-Cl | OCH₂ |
| 6,8-diF | 4-Cl | OCH₂ |
| 7,8-diF | 4-Cl | OCH₂ |
| 2-F | 4-F | C≡C |
| 3-F | 4-F | C≡C |
| 5-F | 4-F | C≡C |
| 6-F | 4-F | C≡C |
| 7-F | 4-F | C≡C |
| 8-F | 4-F | C≡C |
| 2-Cl | 4-F | C≡C |
| 3-Cl | 4-F | C≡C |
| 5-Cl | 4-F | C≡C |
| 6-Cl | 4-F | C≡C |
| 7-Cl | 4-F | C≡C |
| 8-Cl | 4-F | C≡C |
| 2-CF₃ | 4-F | C≡C |
| 3-CF₃ | 4-F | C≡C |
| 5-CF₃ | 4-F | C≡C |
| 6-CF₃ | 4-F | C≡C |
| 7-CF₃ | 4-F | C≡C |
| 8-CF₃ | 4-F | C≡C |
| 2-Me | 4-F | C≡C |
| 3-Me | 4-F | C≡C |
| 5-Me | 4-F | C≡C |
| 6-Me | 4-F | C≡C |
| 7-Me | 4-F | C≡C |
| 8-Me | 4-F | C≡C |
| 2-OMe | 4-F | C≡C |
| 2-OEt | 4-F | C≡C |
| 2-O-iPr | 4-F | C≡C |
| 2-O-iBu | 4-F | C≡C |
| 2-O-nBu | 4-F | C≡C |
| 5,7-diCl | 4-F | C≡C |
| 6,7-diCl | 4-F | C≡C |
| 5,8-diCl | 4-F | C≡C |
| 7,8-diCl | 4-F | C≡C |
| 5,7-diCl | 4-F | C≡C |
| 6,8-diF | 4-F | C≡C |
| 7,8-diF | 4-F | C≡C |
| 2-Me | 4-Cl | C≡C |
| 3-Me | 4-Cl | C≡C |
| 5-Me | 4-Cl | C≡C |
| 6-Me | 4-Cl | C≡C |
| 7-Me | 4-Cl | C≡C |
| 8-Me | 4-Cl | C≡C |
| 2-OMe | 4-Cl | C≡C |
| 2-OEt | 4-Cl | C≡C |
| 2-O-iPr | 4-Cl | C≡C |
| 2-O-iBu | 4-Cl | C≡C |
| 2-O-nBu | 4-Cl | C≡C |
| 5,7-diCl | 4-Cl | C≡C |
| 6,7-diCl | 4-Cl | C≡C |
| 5,8-diCl | 4-Cl | C≡C |
| 7,8-diCl | 4-Cl | C≡C |
| 5,7-diCl | 4-Cl | C≡C |
| 6,8-diF | 4-Cl | C≡C |
| 7,8-diF | 4-Cl | C≡C |

TABLE 19

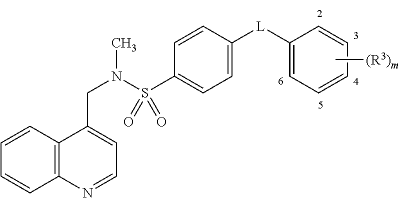

(R³)ₘ represent one or a combination of substituents and no (R³)ₘ
substituents is represented by a dash “—”.

| (R³)ₘ | L |
|---|---|
| — | CH₂ |
| 2-F | CH₂ |
| 3-F | CH₂ |
| 4-F | CH₂ |
| 2-Cl | CH₂ |
| 3-Cl | CH₂ |
| 4-Cl | CH₂ |
| 2-Br | CH₂ |
| 3-Br | CH₂ |
| 4-Br | CH₂ |
| 2-I | CH₂ |
| 3-I | CH₂ |
| 4-I | CH₂ |
| 2-Me | CH₂ |
| 3-Me | CH₂ |
| 4-Me | CH₂ |
| 2-OMe | CH₂ |
| 3-OMe | CH₂ |
| 4-OMe | CH₂ |
| 2-CF₃ | CH₂ |
| 3-CF₃ | CH₂ |
| 4-CF₃ | CH₂ |
| 2-OCF₃ | CH₂ |
| 3-OCF₃ | CH₂ |
| 4-OCF₃ | CH₂ |
| 2-NO₂ | CH₂ |
| 3-NO₂ | CH₂ |
| 4-NO₂ | CH₂ |
| 2-CO₂Me | CH₂ |
| 3-CO₂Me | CH₂ |
| 4-CO₂Me | CH₂ |
| 2-SMe | CH₂ |
| 3-SMe | CH₂ |
| 4-SMe | CH₂ |
| 2-SO₂Me | CH₂ |
| 3-SO₂Me | CH₂ |
| 4-SO₂Me | CH₂ |
| 2-Ph | CH₂ |
| 3-Ph | CH₂ |
| 4-Ph | CH₂ |
| 2-OPh | CH₂ |
| 3-OPh | CH₂ |
| 4-OPh | CH₂ |
| — | OCH₂ |
| 2-F | OCH₂ |
| 3-F | OCH₂ |
| 4-F | OCH₂ |
| 2-Cl | OCH₂ |
| 3-Cl | OCH₂ |
| 4-Cl | OCH₂ |
| 2-Br | OCH₂ |
| 3-Br | OCH₂ |
| 4-Br | OCH₂ |
| 2-I | OCH₂ |
| 3-I | OCH₂ |
| 4-I | OCH₂ |
| 2-Me | OCH₂ |
| 3-Me | OCH₂ |
| 4-Me | OCH₂ |
| 2-OMe | OCH₂ |
| 3-OMe | OCH₂ |
| 4-OMe | OCH₂ |
| 2-CF₃ | OCH₂ |
| 3-CF₃ | OCH₂ |
| 4-CF₃ | OCH₂ |
| 2-OCF₃ | OCH₂ |
| 3-OCF₃ | OCH₂ |
| 4-OCF₃ | OCH₂ |
| 2-NO₂ | OCH₂ |
| 3-NO₂ | OCH₂ |
| 4-NO₂ | OCH₂ |
| 2-CO₂Me | OCH₂ |
| 3-CO₂Me | OCH₂ |
| 4-CO₂Me | OCH₂ |
| 2-SMe | OCH₂ |
| 3-SMe | OCH₂ |
| 4-SMe | OCH₂ |
| 2-SO₂Me | OCH₂ |
| 3-SO₂Me | OCH₂ |
| 4-SO₂Me | OCH₂ |
| 2-Ph | OCH₂ |
| 3-Ph | OCH₂ |
| 4-Ph | OCH₂ |
| 2-OPh | OCH₂ |
| 3-OPh | OCH₂ |
| 4-OPh | OCH₂ |
| — | C≡C |
| 2-F | C≡C |
| 3-F | C≡C |
| 4-F | C≡C |
| 2-Cl | C≡C |
| 3-Cl | C≡C |
| 4-Cl | C≡C |
| 2-Br | C≡C |
| 3-Br | C≡C |
| 4-Br | C≡C |
| 2-I | C≡C |
| 3-I | C≡C |
| 4-I | C≡C |
| 2-Me | C≡C |
| 3-Me | C≡C |
| 4-Me | C≡C |
| 2-OMe | C≡C |
| 3-OMe | C≡C |
| 4-OMe | C≡C |
| 2-CF₃ | C≡C |
| 3-CF₃ | C≡C |
| 4-CF₃ | C≡C |
| 2-OCF₃ | C≡C |
| 3-OCF₃ | C≡C |
| 4-OCF₃ | C≡C |
| 2-NO₂ | C≡C |
| 3-NO₂ | C≡C |
| 4-NO₂ | C≡C |
| 2-CO₂Me | C≡C |
| 3-CO₂Me | C≡C |
| 4-CO₂Me | C≡C |
| 2-SMe | C≡C |
| 3-SMe | C≡C |
| 4-SMe | C≡C |
| 2-SO₂Me | C≡C |
| 3-SO₂Me | C≡C |
| 4-SO₂Me | C≡C |
| 2-Ph | C≡C |
| 3-Ph | C≡C |
| 4-Ph | C≡C |
| 2-OPh | C≡C |
| 3-OPh | C≡C |
| 4-OPh | C≡C |

TABLE 20

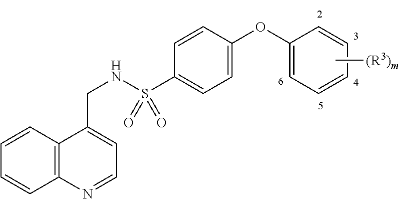

(R³)ₘ represents one or a combination of substituents.
(R³)ₘ
2-CN
3-CN
4-CN
2-CO₂Me
3-CO₂Me
4-CO₂Me
2-CONHMe
3-CONHMe
4-CONHMe
2-CONMe₂
3-CONMe₂
4-CONMe₂
2-NO₂
3-NO₂
4-NO₂
2-SMe
3-SMe
4-SMe
2-SOMe
3-SOMe
4-SOMe
2-SO₂Me
3-SO₂Me
4-SO₂Me
2-SCF₂H
3-SCF₂H
4-SCF₂H
2-SCF₃
3-SCF₃
4-SCF₃
NMe₂
NMe₂
NMe₂
SO₂NMe₂
SO₂NMe₂
SO₂NMe₂
2-C≡CH
3-C≡CH
4-C≡CH
2-C≡CPh
3-C≡CPh
4-C≡CPh
2-CN, 3-Cl
2-CN, 4-Cl
2-CN, 5-Cl
2-CN, 6-Cl
2-Cl, 3-CN
2-Cl, 4-CN
2-Cl, 5-CN
2-Cl, 6-CN
3-CN, 2-Cl
3-CN, 4-Cl
3-CN, 5-Cl
3-CN, 6-Cl
4-CN, 2-Cl
4-CN, 3-Cl
2-CN, 3-F
2-CN, F—Cl
2-CN, F—Cl
2-CN, F—Cl
2-F, 3-CN
2-F, 4-CN
2-F, 5-CN
2-F, 6-CN
3-CN, 2-F
3-CN, 4-F
3-CN, 5-F
3-CN, 6-F
4-CN, 2-F
4-CN, 3-F
2-CO₂Me, 3-Cl
2-CO₂Me, 4-Cl
2-CO₂Me, 5-Cl
2-CO₂Me, 6-Cl
2-Cl, 3-CO₂Me
2-Cl, 4-CO₂Me
2-Cl, 5-CO₂Me
2-Cl, 6-CO₂Me
3-CO₂Me, 2-Cl
3-CO₂Me, 4-Cl
3-CO₂Me, 5-Cl
3-CO₂Me, 6-Cl
4-CO₂Me, 2-Cl
4-CO₂Me, 3-Cl

A compound of this invention will generally be used as a helminth control active ingredient in a composition, i.e. formulation, with at least one additional component selected from the pharmaceutically or veterinarily acceptable carriers or diluents. The formulation or composition ingredients are selected to be consistent with the physical properties of the active ingredient, mode of administration and factors such as the type of animal to be treated.

The compounds of Formula 1 are preferably employed in unmodified form or preferably together with the adjuvants conventionally used in the art of pharmaceutical or veterinary formulation and may therefore be processed in a known manner to give, for example, emulsifiable concentrates, directly dilutable solutions, dilute emulsions, soluble powders, granules or microencapsulations in polymeric substances. As with the compositions, the methods of application are selected in accordance with the intended objectives and the prevailing circumstances.

Applications in the veterinary sector are by conventional means such as by enteral administration in the form of, for example, tablets including effervescent tablets, capsules, micro-capsules, drinks, drenching preparations (solutions, emulsions, suspensions), granulates, pastes, powders, boli, food additives or suppositories; or by parenteral administration, such as by injection (including intramuscular, subcutaneous, intravenous, intraperitoneal) or implants; by nasal administration; by topical administration, for example, in the form of immersion or dipping, spraying, washing, coating with powder, or application to a small area of the animal via a pour-on formulations, and through articles such as neck collars, ear tags, tail bands, limb bands or halters which comprise compounds or compositions of the present invention.

The compounds of the present invention may be administered in a controlled release form, e.g., in a subcutaneous slow release formulation.

The formulation, i.e. the agents, preparations or compositions containing the active ingredient of Formula 1, or combinations of these active ingredients with other active ingredients, and optionally a solid or liquid adjuvant, are produced in a manner known in the art, for example by intimately mixing and/or grinding the active ingredients with spreading compositions, for example with solvents, solid carriers, and optionally surface-active compounds (surfactants).

The solvents in question may be: alcohols, such as ethanol, propanol or butanol, and glycols and their ethers and esters, such as propylene glycol, dipropylene glycol ether, ethylene glycol, ethylene glycol monomethyl or -ethyl ether, ketones, such as cyclohexanone, isophorone or diacetanol alcohol, strong polar solvents, such as N-methyl-2-pyrrolidone, dimethyl sulfoxide or dimethylformamide, or water, vegetable oils, such as rape, castor, coconut, or soybean oil, and also, if appropriate, silicone oils.

For parenteral administration including intravenous, intramuscular and subcutaneous injection, a compound of the present invention can be formulated in suspension, solution or emulsion in oily or aqueous vehicles, and may contain adjuncts such as suspending, stabilizing and/or dispersing agents. The compounds of the present invention may also be formulated for bolus injection or continuous infusion. Pharmaceutical and veterinary compositions for injection include aqueous solutions of water-soluble forms of active ingredients (e.g., a salt of an active compound), preferably in physiologically compatible buffers containing other excipients or auxiliaries as are known in the art of pharmaceutical and veterinary formulation. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Formulations for injection may be presented in unit dosage form, e.g., in ampoules or in multi-dose containers. Alternatively, the active ingredient may be in powder form for constitution with a suitable vehicle, e.g., sterile, pyrogen-free water, before use.

In addition to the formulations described above, the compounds of the present invention may also be formulated as a depot preparation. Such long acting formulations may be administered by implantation (for example, subcutaneously or intramuscularly) or by intramuscular or subcutaneous injection. The compounds of the present invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

For administration by inhalation, the compounds of the present invention can be delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

Compounds of the present invention have been discovered to have favorable pharmacokinetic and pharmacodynamic properties providing systemic availability from oral administration and ingestion. Therefore after ingestion by the animal to be protected, parasiticidally effective concentrations of compounds of the invention in the bloodstream protect the treated animal from blood-sucking pests. Therefore of note is a composition for protecting an animal from an invertebrate parasite pest in a form for oral administration (i.e. comprising, in addition to a parasiticidally effective amount of a compound of the invention, one or more carriers selected from binders and fillers suitable for oral administration and feed concentrate carriers).

For oral administration in the form of solutions (the most readily available form for absorption), emulsions, suspensions, pastes, gels, capsules, tablets, boluses, powders, granules, rumen-retention and feed/water/lick blocks, a compound of the present invention can be formulated with binders/fillers known in the art to be suitable for oral administration compositions, such as sugars and sugar derivatives (e.g., lactose, sucrose, mannitol, sorbitol), starch (e.g., maize starch, wheat starch, rice starch, potato starch), cellulose and derivatives (e.g., methylcellulose, carboxymethylcellulose, ethylhydroxycellulose), protein derivatives (e.g., zein, gelatin), and synthetic polymers (e.g., polyvinyl alcohol, polyvinylpyrrolidone). If desired, lubricants (e.g., magnesium stearate), disintegrating agents (e.g., cross-linked polyvinylpyrrolidinone, agar, alginic acid) and dyes or pigments can be added. Pastes and gels often also contain adhesives (e.g., acacia, alginic acid, bentonite, cellulose, xanthan gum, colloidal magnesium aluminum silicate) to aid in keeping the composition in contact with the oral cavity and not being easily ejected.

If the anthelmintics are present in the form of feed concentrates, then the carriers used are e.g. performance feeds, feed grain or protein concentrates. Such feed concentrates or compositions may contain, apart from the active ingredients, also additives, vitamins, antibiotics, chemotherapeutics or other pesticides, primarily bacteriostats, fungistats, coccidiostats, or even hormone preparations, substances having anabolic action or substances which promote growth, which affect the quality of meat of animals for slaughter or which are beneficial to the organism in another way. If the compositions or the active ingredients of Formula 1 contained therein are added directly to feed or to the drinking troughs, then the formulated feed or drink contains the active ingredients preferably in a concentration of ca. 0.0005 to 0.02% by weight (5-200 ppm).

The compounds of Formula 1 may also be formulated in rectal compositions such as suppositories or retention enemas, using, e.g., conventional suppository bases such as cocoa butter or other glycerides.

Formulations for topical administration are typically in the form of a powder, cream, suspension, spray, emulsion, foam, paste, aerosol, ointment, salve or gel. More typically a topical formulation is a water-soluble solution, which can be in the form of a concentrate that is diluted before use. Parasiticidal compositions suitable for topical administration typically comprise a compound of the present invention and one or more topically suitable carriers. In applications of a parasiticidal composition topically to the exterior of an animal as a line or spot (i.e. “spot-on” treatment), the active ingredient migrates over the surface of the animal to cover most or all of its external surface area. Therefore formulations for topical localized administration often comprise at least one organic solvent to facilitate transport of the active ingredient over the skin and/or penetration into the epidermis of the animal. Carriers in such formulations include propylene glycol, paraffins, aromatics, esters such as isopropyl myristate, glycol ethers, alcohols such as ethanol, n-propanol, 2-octyl dodecanol or oleyl alcohol; solutions in esters of monocarboxylic acids, such as isopropyl myristate, isopropyl palmitate, lauric acid oxalic ester, oleic acid oleyl ester, oleic acid decyl ester, hexyl laurate, oleyl oleate, decyl oleate, caproic acid esters of saturated fatty alcohols of chain length C₁₂-C₁₈; solutions of esters of dicarboxylic acids, such as dibutyl phthalate, diisopropyl isophthalate, adipic acid diisopropyl ester, di-n-butyl adipate or solutions of esters of aliphatic acids, e.g., glycols. It may be advantageous for a crystallization inhibitor or a dispersant known from the pharmaceutical or cosmetic industry also to be present.

The pour-on or spot-on method consists in applying the parasiticidal composition to a specific location of the skin or coat, advantageously to the neck or backbone of the animal. This takes place by applying a swab or spray of the pour-on or spot-on formulation to a relatively small area of the coat, from where the active substance is dispersed almost automatically over wide areas of the fur owing to the spreading nature of the components in the formulation and assisted by the animal's movements. The pour-on formulation is typically applied by pouring in one or several lines or in a spot-on the dorsal midline (back) or shoulder of an animal. More typically, the formulation is applied by pouring it along the back of the animal, following the spine. The formulation can also be applied to the animal by other conventional methods, including wiping an impregnated material over at least a small area of the animal, or applying it using a commercially available applicator, by means of a syringe, by spraying or by using a spray race. Pour-on or spot-on formulations suitably contain carriers, which promote rapid dispersement over the skin surface or in the coat of the host animal, and are generally regarded as spreading oils. Suitable carriers are, for example, oily solutions; alcoholic and isopropanolic solutions such as solutions of 2-octyldodecanol or oleyl alcohol; solutions in esters of monocarboxylic acids, such as isopropyl myristate, isopropyl palmitate, lauric acid oxalate, oleic acid oleyl ester, oleic acid decyl ester, hexyllaurate, oleyl oleate, decyl oleate, capric acid esters of saturated fat alcohols of chain length C₁₂-C₁₈; solutions of esters of dicarboxylic acids, such as dibutyl phthalate, diisopropyl isophthalate, adipic acid diisopropyl ester, di-n-butyl adipate or also solutions of esters of aliphatic acids, for example glycols. It may be advantageous for a dispersing agent to be additionally present, such as one known from the pharmaceutical or cosmetic industry. Examples are 2-pyrrolidone, 2-(N-alkyl)pyrrolidone, acetone, polyethylene glycol and the ethers and esters thereof, propylene glycol or synthetic triglycerides.

The oily solutions include, for example, vegetable oils such as olive oil, groundnut oil, sesame oil, pine oil, linseed oil or castor oil. The vegetable oils may also be present in epoxidised form. Paraffins and silicone oils may also be used.

A pour-on or spot-on formulation generally contains 1 to 20% by weight of a compound of Formula 1, 0.1 to 50% by weight of dispersing agent and 45 to 98.9% by weight of solvent.

The pour-on or spot-on method is especially advantageous for use on herd animals such as cattle, horses, sheep or pigs, in which it is difficult or time-consuming to treat all the animals orally or by injection. Because of its simplicity, this method can of course also be used for all other animals, including individual domestic animals or pets, and is greatly favoured by the keepers of the animals, as it can often be carried out without the specialist presence of the veterinarian.

The formulations of this invention typically include an antioxidant, such as BHT (butylated hydroxytoluene). The antioxidant is generally present in amounts of at 0.1-5% (wt/vol).

The compositions may also contain further additives, such as stabilisers, e.g. where appropriate epoxidised vegetable oils (epoxidised coconut oil, rapeseed oil, or soybean oil); antifoams, e.g. silicone oil, preservatives (e.g. methylparaben and propylparaben), viscosity regulators, thickeners (e.g. carbomers, corn starch, polyethylene, polyvinylpyrrolidones, edible clay or xanthan gum) binders and tackifiers or other active ingredients to achieve special effects.

Further biologically active substances or additives, which are neutral towards the compounds of Formula 1 and do not have a harmful effect on the host animal to be treated, as well as mineral salts or vitamins, may also be added to the described compositions. As a rule, the anthelmintic compositions according to the invention contain 0.1 to 99% by weight, especially 0.1 to 95% by weight of active ingredient of Formula 1, 99.9 to 1% by weight, especially 99.8 to 5% by weight of a solid or liquid admixture, including 0 to 25% by weight, especially 0.1 to 25% by weight of a surfactant.

Whereas it is preferred to formulate commercial products as concentrates, the end user will normally use dilute formulations.

In each of the methods according to the invention for pest control or in each of the pest control compositions according to the invention, the active ingredients of Formula 1 can be used in all of their steric configurations or in mixtures thereof.

The invention also includes a method of prophylactically protecting warm-blooded animals, especially productive-livestock, domestic animals and pets, against parasitic helminths, which is characterised in that the active ingredients of the formula or the active ingredient formulations prepared therefrom are administered to the animals as an additive to the feed, or to the drinks or also in solid or liquid form, orally or by injection or parenterally. The invention also includes the compounds of Formula 1 according to the invention for usage in one of the said methods.

In the following Examples, all formulations are prepared in conventional ways. Compound numbers refer to compounds in Index Tables A and B. Without further elaboration, it is believed that one skilled in the art using the preceding description can utilize the present invention to its fullest extent. The following Examples are, therefore, to be construed as merely illustrative, and not limiting of the disclosure in any way whatsoever.

Percentages are by weight except where otherwise indicated.

### Example A

| Granulate | a) | b) |
|---|---|---|
| compound 2 | 5% | 10% |
| kaolin | 94% | — |
| highly dispersed silicic acid | 1% | — |
| attapulgite | — | 90% |

The active ingredient is dissolved in methylene chloride, sprayed onto the carrier and the solvent subsequently concentrated by evaporation under vacuum. Granulates of this kind can be mixed with the animal feed.

### Example B

Dust Free Granulate

| | |
|---|---|
| compound 5 | 3% |
| polyethylene glycol (molecular weight 200) | 3% |
| kaolin | 94% |

The finely ground active ingredient is evenly applied in a mixer to the kaolin which has been moistened with polyethylene glycol. In this way, dust-free coated granules are obtained.

### Example C

Tablets or Boli

| | |
|---|---|
| 1) compound 9 | 33.00% |
| 1) methyl cellulose | 0.80% |
| 1) highly dispersed silicic acid | 0.80% |
| 1) corn starch | 8.40% |
| 2) crystalline lactose | 22.50% |
| 2) corn starch | 17.00% |
| 2) microcrystalline celluose | 16.50% |
| 2) magnesium stearate | 1.00% |

1) Methyl cellulose is stirred into water. After the material has swollen, silicic acid is stirred in and the mixture homogeneously suspended. The active ingredient and the corn starch are mixed. The aqueous suspension is worked into this mixture and kneaded to a dough. The resulting mass is granulated through a 12 M sieve and dried.

2) All 4 excipients are mixed thoroughly.

3) The preliminary mixes obtained according to 1 and 2 are mixed and pressed into tablets or boli.

Injectable: Oily Vehicle (slow release)

| | | |
|---|---|---|
| 1) compound 15 | 0.1-1.0 | g |
| 1) groundnut oil | ad 100 | mL |
| 2) compound 18 | 0.1-1.0 | g |
| 2) sesame oil | ad 100 | mL |

The active ingredient is dissolved in part of the oil while stirring and, if required, with gentle heating, then after cooling made up to the desired volume and sterile-filtered through a suitable membrane filter with a pore size of 0.22 μm.

“ad” means enough of this component is added to a mixture of the other components to make a specified total volume (100 mL in this case) for the formulation.

### Example E

Injectable: Water-Miscible Solvent (average rate of release)

| | | |
|---|---|---|
| 1) compound 19 | 0.1-1.0 | g |
| 1) 4-hydroxymethyl-1,3-dioxolane (glycerol | 40 | g |
| formal) | | |
| 1) 1,2-propanediol | ad 100 | mL |
| 2) compound 20 | 0.1-1.0 | g |
| 2) glycerol dimethyl ketal | 40 | g |
| 2) 1,2-propanediol | ad 100 | mL |

The active ingredient is dissolved in part of the solvent while stirring, made up to the desired volume and sterile-filtered through a suitable membrane filter with a pore size of 0.22 μm.

### Example F

Injectable: Aqueous Solubilisate (rapid release)

| | | |
|---|---|---|
| 1) compound 23 | 0.1-1.0 | g |
| 1) polyethoxylated castor oil (40 ethylene oxide units) | 10 | g |
| 1) 1,2-propanediol | 20 | g |
| 1) benzyl alcohol | 1 | g |
| 1) water for injection | ad 100 | mL |
| 2) compound 32 | 0.1-1.0 | g |
| 2) polyethoxylated sorbitan monooleate (20 ethylene oxide | 8 | g |
| units) | | |
| 2) 4-hydroxymethyl-1,3-dioxolane (glycerol formal) | 20 | g |
| 2) benzyl alcohol | 1 | g |
| 2) water for injection | ad 100 | mL |

The active ingredient is dissolved in the solvents and the surfactant, and made up with water to the desired volume. The solution is then sterile-filtered through a suitable membrane filter with a pore size of 0.22 μm.

### Example G

Pour-On

| | | |
|---|---|---|
| 1) compound 34 | 5 | g |
| 1) isopropyl myristate | 10 | g |
| 1) isopropanol | ad 100 | mL |
| 2) compound 2 | 2 | g |
| 2) hexyl laurate | 5 | g |
| 2) medium-chained triglyceride | 15 | g |
| 2) ethanol | ad 100 | mL |
| 3) compound 5 | 2 | g |
| 3) oleyl oleate | 5 | g |
| 3) N-methyl-pyrrolidone | 40 | g |
| 3) isopropanol | ad 100 | mL |

The aqueous systems may also preferably be used for oral and/or intraruminal application.

In general for veterinary use, a compound of Formula 1, an N-oxide, or salt thereof, is administered in a parasiticidally effective amount to an animal to be protected from helminth parasite pests. A parasiticidally effective amount is the amount of active ingredient needed to achieve an observable effect diminishing the occurrence or activity of the target helminth parasite pest. One skilled in the art will appreciate that the parasitically effective dose can vary for the various compounds and compositions of the present invention, the desired parasitical effect and duration, the target helminth pest species, the animal to be protected, the mode of application and the like, and the amount needed to achieve a particular result can be determined through simple experimentation.

For oral administration to homeothermic animals, the daily dosage of a compound of the present invention typically ranges from about 0.01 mg/kg to about 100 mg/kg, more typically from about 0.5 mg/kg to about 100 mg/kg, of animal body weight. For topical (e.g., dermal) administration, dips and sprays typically contain from about 0.5 ppm to about 5000 ppm, more typically from about 1 ppm to about 3000 ppm, of a compound of the present invention.

Compounds of the present invention have activity on members of the classes Nematoda (roundworms), Tematoda (flukes), Acanthocephala and Cestoda (tapeworms). Important helminths are those that cause serious diseases of mammals and poultry, e.g. sheep, pigs, goats, cattle, horses, donkeys, dogs, cats, guinea-pigs and birds. Typical nematodes of this indication are: Haemonchus, Trichostrongylus, Teladorsagia, Dirofilaria, Ostertagia, Nematodirus. Cooperia, Ascaris, Bunostonum, Oesophagostonum, Charbertia, Trichuris, Strongylus, Trichonema, Dictyocaulus, Capillaria, Heterakis, Toxocara, Ascaridia, Oaxuris, Ancylostoma, Uncinaria, Toxascaris and Parascaris. The trematodes include the family of Fasciolideae, especially Fasciola hepatica. Certain pests of the species Nematodirus, Cooperia and Oesophagostonum infest the intestinal tract of the host animal, while others of the species Haemonchus and Ostertagia are parasitic in the stomach and those of the species Dictyocaulus are parasitic in the lung tissue. Parasites of the families Filariidae and Setariidae may be found in the internal cell tissue and in the organs, e.g. the heart, the blood vessels, the lymph vessels and the subcutaneous tissue. A notable parasite is the heartworm of the dog, Dirofilaria immitis. Important pests of the class Cestoda (tapeworms) include, the families Mesocestoidae, especially of the genus Mesocestoides, in particular M. lineatus; Dilepidide, especially Dipylidium caninum, Joyeuxiella spp., in particular Jovetxiella pasquali, and Diplopylidium spp., and Taeniidae, especially Taenia pisiformis, Taenia cervi, Taenia ovis, Taneia hydatigena, Taenia multiceps, Taenia taeniaeformis, Taenia serialis, and Echinocuccus spp., most preferably Taneia hydatigena, Taenia ovis, Taenia multiceps, Taenia serialis; Echinocuccus granulosus and Echinococcus multilocularis, as well as Multiceps multiceps. Another notable parasite is Anoplocephala perfoliata in horses.

The compounds of the present invention may be suitable for the control of human pathogenic parasites. Of these, typical representatives that appear in the digestive tract are those of the species Ancylostoma, Necator, Ascaris, Strongvloides, Trichinella, Capillaria, Trichuris and Enterobius. The compounds of the present invention may also be effective against parasites of the species Wuchereria, Brugia, Onchocerca and Loa from the family of Filariidae, which appear in the blood, in the tissue and in various organs, and also against Dracunculus and parasites of the species Strongvloides and Trichinella, which infect the gastrointestinal tract in particular.

Numerous other Helminth genera and species are known to the art, and are also contemplated to be treated by the compounds of the invention. These are enumerated in great detail in Textbook of Veterinaty Clinical Parasitology, Volume 1, Helminths, E. J. L. Soulsby, F. A. Davis Co., Philadelphia, Pa.; Helminths, Arthropods and Protozoa, (6^{th} Edition of Monnig's Veterinary Helminthology and Entomology), E. J. L. Soulsby, The Williams and Wilkins Co., Baltimore, Md.

Compounds and compositions of the present invention are suitable for combating parasites that infest animal subjects including those in the wild, livestock and agricultural working animals such as cattle, sheep, goats, horses, pigs, donkeys, camels, bison, buffalos, rabbits, hens, turkeys, ducks and geese (e.g., raised for meat, milk, butter, eggs, fur, leather, feathers and/or wool). By combating parasites, fatalities and performance reduction (in terms of meat, milk, wool, skins, eggs etc.) are reduced, so that applying a composition comprising a compound of the present invention allows more economic and simple husbandry of animals.

Compounds and compositions of the present invention are especially suitable for combating parasites that infest companion animals and pets (e.g., dogs, cats and pet birds), research and experimental animals (e.g., hamsters, guinea pigs, rats and mice), as well as animals raised for/in zoos, wild habitats and/or circuses.

In an embodiment of this invention, the animal is preferably a vertebrate, and more preferably a mammal or avian. In a particular embodiment, the animal subject is a mammal (including great apes, such as humans). Other mammalian subjects include primates (e.g., monkeys), bovine (e.g., cattle or dairy cows), porcine (e.g., hogs or pigs), ovine (e.g., goats or sheep), equine (e.g., horses), canine (e.g., dogs), feline (e.g., house cats), camels, deer, donkeys, bison, buffalos, antelopes, rabbits, and rodents (e.g., guinea pigs, squirrels, rats, mice, gerbils, and hamsters). Avians include Anatidae (swans, ducks and geese), Columbidae (e.g., doves and pigeons), Phasianidae (e.g., partridges, grouse and turkeys), Thesienidae (e.g., domestic chickens), Psittacines (e.g., parakeets, macaws, and parrots), game birds, and ratites (e.g., ostriches).

Birds treated or protected by the inventive compounds can be associated with either commercial or noncommercial aviculture. These include Anatidae, such as swans, geese, and ducks, Columbidae, such as doves and domestic pigeons, Phasianidae, such as partridge, grouse and turkeys, Thesienidae, such as domestic chickens, and Psittacines, such as parakeets, macaws, and parrots raised for the pet or collector market, among others.

As a consequence of the above details, a further essential aspect of the present invention relates to combination preparations for the control of parasites on warm-blooded animals, characterised in that they contain, in addition to a compound of Formula 1, at least one further active ingredient having the same or different sphere of activity and at least one physiologically acceptable carrier. The present invention is not restricted to two-fold combinations.

The compounds of Formula 1 according to the invention may be used alone or in combination with other biocides. They may be combined with pesticides having the same sphere of activity e.g. to increase activity, or with substances having another sphere of activity e.g. to broaden the range of activity. It can also be sensible to add so-called repellents if the formulation is applied externally. They can also be used in combination with antibacterial compositions. Compounds which attack the juvenile stages of parasites may be very advantageous to add to those that function primarily as adulticides. In this way, the greatest range of those parasites that produce great economic damage will be covered. Moreover, this action will contribute substantially to avoiding the formation of resistance. Many combinations may also lead to synergistic effects, i.e. the total amount of active ingredient can be reduced, which is desirable from an ecological point of view. Preferred groups of combination partners and especially preferred combination partners are named in the following, whereby combinations may contain one or more of these partners in addition to a compound of Formula 1.

Of note are additional biologically active compounds or agents selected from art-known anthelmintics, such as, for example, macrocyclic lactones including but not limited to avermectins and derivatives thereof (e.g., ivermectin, moxidectin, milbemycin), benzimidazoles (e.g., albendazole, triclabendazole, cambendazole, fenbendazole, flubendazole, mebendazole, oxfendazole, oxibendazole, parbendazole), salicylanilides (e.g., closantel, oxyclozanide), substituted phenols (e.g., nitroxynil), tetrahydropyrimidines (e.g., pyrantel pamoate, oxantel, morantel), imidazothiazoles (e.g., levamisole, tetramizole) and praziquantel. Additonal art-known anthelmintics include analogs and derivatives of the paraherquamide/marcfortine class, nitroscanate, and cyclic depsipeptides, e.g., emodepside.

Of particular note are biologically active compounds or agents useful in the compositions of the present invention selected from the antiparasitic class of avermectin compounds mentioned above. The avermectin family of compounds is a series of very potent antiparasitic agents known to be useful against a broad spectrum of endoparasites and ectoparasites in mammals. A notable compound in this class for use within the scope of the present invention is ivermectin. Ivermectin is a semi-synthetic derivative of avermectin and is generally produced as a mixture of at least 80% 22,23-dihydroavermectin B₁ₐ and less than 20% 22,23-dihydroavermectin B_{1b}.

Other notable avermectins are abamectin, doramectin, dimadectin, latidectin, lepimectin, selamectin, milbemycin and derivatives thereof including but not limited to milbemectin, moxidectin, nemadectin and milbemycin D, emamectin, and eprinomectin. Eprinomectin is chemically known as 4″-epi-acetylamino-4″-deoxy-avermectin B₁. Eprinomectin was specifically developed to be used in all cattle classes and age groups. It was the first avermectin to show broad-spectrum activity against both endo- and ecto-parasites while also leaving minimal residues in meat and milk. It has the additional advantage of being highly potent when delivered topically.

Also of note are nodulisporic acids and their derivatives, known in the art as a class of compounds that are potent endo- and ectopantiparasitic agents. The isolation and purification of three naturally occurring nodulisporic acids are disclosed in U.S. Pat. No. 5,399,582. Derivatives of these compounds are described in WO 96/29073 and U.S. Pat. Nos. 5,945,317, 5,962,499, 5,834,260, 6,399,796, 6,221,894, 6,136,838, 5,595,991, 5,299,582, and 5,614,546.

The composition of the present invention optionally comprises combinations of one or more of the following antiparasite compounds: imidazo[1,2-b]pyridazine compounds as described by U.S. application Ser. No. 11/019,597, filed on Dec. 22, 2004, and published on Aug. 18, 2005 as U.S. 2005-0182059A1; trifluoromethanesulfonanilide oxime ether derivatives, as described by U.S. application Ser. No. 11/231,423, filed on Sep. 21, 2005, now U.S. Pat. No. 7,312,248; and N-[(phenyloxy)phenyl]-1,1,1-trifluoromethanesulfonamide and N-[(phenylsulfanyl)phenyl]-1,1,1-trifluoromethanesulfonamide derivatives, as described by U.S. Provisional Application Ser. No. 60/688,898, filed on Jun. 9, 2005, and published as US 2006-0281695A1 on Dec. 14, 2006.

The compositions of the present invention can also further comprise a flukicide. Suitable flukicides include, for example, triclabendazole, fenbendazole, albendazole, clorsulon and oxibendazole. It will be appreciated that the above combinations can further include combinations of antibiotic, antiparasitic and anti-fluke active compounds.

In addition to the above combinations, it is also contemplated to provide combinations of the inventive methods and compounds, as described herein, with other animal health remedies such as trace elements, anti-inflammatories, anti-infectives, hormones, dermatological preparations, including antiseptics and disinfectants, and immunobiologicals such as vaccines and antisera for the prevention of disease.

For example, such anti-infectives include one or more antibiotics that are optionally co-administered during treatment using the inventive compounds or methods, e.g., in a combined composition and/or in separate dosage forms. Art-known antibiotics suitable for this purpose include, for example, those listed herein below.

Useful antibiotics are chloramphenicol analogs such as florfenicol, also known as D-(threo)-1-(4-methylsulfonylphenyl)-2-dichloroacetamido-3-fluoro-1-propanol. Other notable chloramphenicol analogs include thiamphenicol and D-(threo)-1-(4-methylsulfonyphenyl)-2-difluoroacetamido-3-fluoro-1-propanol. Other florfenicol analogs and/or prodrugs have been disclosed and such analogs also can be used in the compositions and methods of the present invention (e.g., U.S. Patent Application Publication No. 2004/0082553, now U.S. Pat. No. 7,041,670, U.S. patent application Ser. No. 11/016,794, now U.S. Pat. No. 7,153,842, and U.S. application Ser. No. 11/018,156, filed on Dec. 21, 2004, now U.S. Pat. No. 7,361,689).

Other useful antibiotics for use in the present invention are macrolide antibiotics such as tilmicosin and tulathromycin.

Other useful macrolide antibiotics include compounds from the class of ketolides, or, more specifically, the azalides. Such compounds are described in, for example, U.S. Pat. No. 6,514,945, U.S. Pat. No. 6,472,371, U.S. Pat. No. 6,270,768, U.S. Pat. No. 6,437,151, U.S. Pat. No. 6,271,255, U.S. Pat. No. 6,239,112, U.S. Pat. No. 5,958,888, U.S. Pat. No. 6,339,063 and U.S. Pat. No. 6,054,434.

Other antibiotics may include β-lactams such as cephalosporins, e.g., ceftiofur, cefquinome, etc., and penicillins, e.g., penicillin, ampicillin, amoxicillin, or a combination of amoxicillin with clavulanic acid or other beta lactamase inhibitors.

Another useful antibiotic class includes the fluoroquinolones, such as, for example, enrofloxacin, danofloxacin, difloxacin, orbifloxacin and marbofloxacin.

Other useful antibiotics include the tetracyclines, particularly chlortetracycline and oxytetracycline.

Representative compounds of this invention prepared by the methods described herein are shown in Index Tables A-B. See Index Table C for ¹H NMR data. For mass spectral data (AP⁺ (M+1)), the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺). The alternate molecular ion peaks (e.g., M+2 or M+4) that occur with compounds containing multiple halogens are not reported. The reported M+1 peaks were observed by mass spectrometry using atmospheric pressure chemical ionization (AP⁺) or electrospray ionization (ESI).

The following abbreviations are used in the Index Tables which follow: Cmpd means Compound, Me means methyl, Et means ethyl, OMe means methoxy, CN means cyano, NO₂ means nitro and C(O) means carbonyl and SO₂ means sulfonyl.

INDEX TABLE A

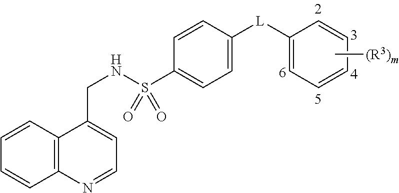

| | | | | AP+ |
|---|---|---|---|---|
| Cmpd | L | R³ | m.p. (° C.) | (M + 1) |
| 1 | O | 2-Cl-6-NO₂ | 116-117 | |
| 2 | O | 3-Cl-2-CN | 238-239 | |
| 4 | C(O) | H | 115-116 | |
| 5 | CH₂ | H | 106-107 | |
| 6 | OCH₂ | H | 105-106 | |
| 7 | OCH₂ | 2,4-diCl | 145-146 | |
| 8 | C(O)NH | 2,4-diCl | 189-190 | |
| 9 | O | 4-CN | 159-160 | |
| 10 | S | 2,4-diCl | 158-159 | |
| 11 | SO₂ | 4-Cl | 185-186 | |
| 12 | C(O)NH | H | 228-229 | |
| 13 | O | 3-Cl-4-CN | 196-197 | |
| 14 | O | 4-CN-2-OMe | 153-154 | |
| 15 | O | 2-Cl-4-CN | 85-88 | |
| 16 | O | 2-CO₂Me | 163-166 | |
| 17 | O | 4-CO₂Me | 73-76 | |
| 18 | O | 2-CN | 82-84 | |
| 19 | CH₂O | H | 140-143 | |
| 20 | C≡C | 4-Cl | 197-199 | |
| 21 | C≡C | 2-Cl | 142-145 | |
| 22 | CH₂CH₂ | 2-Cl | 102-104 | |
| 23 | O | 3-CN | 82-84 | |
| 24 | CH₂CH₂ | 4-Cl | * | 437 |
| 25 | O | 3-CO₂Me | 162-165 | |
| 26 | O | 4-Cl-2-CO₂Me | 156-157 | |
| 27 | O | 2-Cl-4-CO₂Me | 141-143 | |
| 28 | C≡C | H | 125-129 | |
| 29 | O | 2-Br-4-CN | 161-164 | |
| 31 | O | 4-Br-2-CN | 157-159 | |
| 32 | C≡C | 2,4-diCl | 209-212 | |
| 34 | CH₂O | 4-Cl | * | 439 |
| 35 | O | 4-Cl-2-CN | 85-88 | |
| 38 | CH₂CH₂ | H | | 403 |
| 39 | CH₂CH₂ | 2,4-diCl | 153-156 | |
| 47 | CH═CH | H | 172-173 | |
| 48 | O | 4-CO₂H | 204-207 | |
| 49 | O | 4-C(O)NMe₂ | 79-82 | |
| 51 | O | 4-C(O)NHMe | 199-201 | |
| 52 | CH₂ | 4-CF₃ | | 457 |
| 53 | NHC(O) | H | 245-247 | |
| 54 | O | 2-C(O)NHMe | 85-88 | |
| 55 | NHC(O) | 2,4-diCl | 204-207 | |
| 56 | NHC(O) | 2-Cl | 214-217 | |
| 57 | O | 2-C(O)NMe₂ | 73-76 | |
| 58 | NHC(O) | 4-Cl | 216-218 | |
| 59 | CH₂ | 4-F | 124-125 | |
| 60 | CH₂ | 2,4-diF | 128-129 | |
| 61 | NHC(O) | 4-OMe | 235-237 | |
| 62 | O | 3-C(O)NHMe | 97-100 | |
| 63 | O | 3-C(O)NMe₂ | 66-69 | |
| 65 | CH₂ | 3-CF₃ | 93-94 | |
| 66 | NHSO₂ | 4-Cl | 154-157 | |
| 67 | NHSO₂ | 4-CH₃ | 112-115 | |
| 68 | NHSO₂ | H | 111-114 | |
| 69 | NHC(O) | 2-OMe | 199-202 | |
| 70 | NH | H | 119-120 | |
| 71 | NH | 4-Cl | 115-116 | |
| 76 | CHF | H | 120-121 | |
| 77 | CHOH | H | 105-107 | |
| 78 | C≡C | 2-F | 154-156 | |
| 79 | C≡C | 4-F | 167-169 | |
| 81 | C≡C | 3-Cl-4-F | 189-191 | |
| 83 | C≡C | 2-Cl-S-F | 182-185 | |
| 84 | C≡C | 3-F | 197-199 | |
| 85 | C≡C | 2,4-diF | 163-166 | |
| 87 | C≡C | 3-Cl-5-F | 168-171 | |
| 89 | C≡C | 2-CF₃ | 201-204 | |
| 90 | NH | 4-F | | 408 |
| 91 | CH₂ | 2-F | | 407 |
| 93 | C≡C | 3-CF₃ | 177-179 | |
| 94 | C≡C | 5-Cl-2-F | 192-195 | |
| 95 | C≡C | 4-Cl-2-F | 208-210 | |
| 96 | C≡C | 2-Cl-5-F | 186-189 | |
| 97 | C≡C | 4-CF₃ | 196-199 | |
| 98 | C≡C | 3-F-5-CF₃ | 182-185 | |
| 102 | NHC(O) | 3-Cl | 200-201 | |
| 103 | NHC(O) | 2-F | 198-200 | |
| 104 | NHC(O) | 2-CF₃ | 194-196 | |
| 105 | NHC(O) | 2-Br | 196-198 | |
| 106 | NHC(O) | 2-OCF₃ | 170-172 | |
| 107 | NHC(O) | 3-OMe | 193-195 | |
| 109 | NHC(O) | 4-CN | 225-228 | |
| 110 | NHC(O) | 2-CN | 150-154 | |

See Index Table C for ¹H NMR data.

INDEX TABLE B

| | | | AP+ |
|---|---|---|---|
| Cmpd | Structure | m.p. (° C.) | (M + 1) |
| 3 | 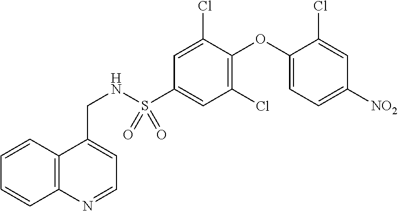 [SMILES: O=[N+]([O-])c1ccc(Oc2c(Cl)cc(S(=O)(=O)NCc3ccnc4ccccc34)cc2Cl)c(Cl)c1] | 198-199 | |
| 30 | 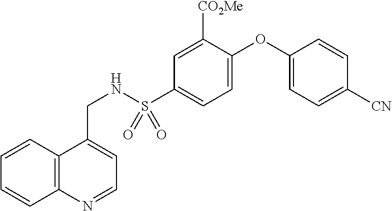 [SMILES: COC(=O)c1cc(S(=O)(=O)NCc2ccnc3ccccc23)ccc1Oc1ccc(C#N)cc1] | 173-175 | |
| 33 | 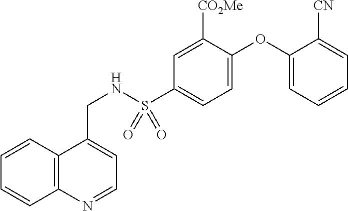 [SMILES: COC(=O)c1cc(S(=O)(=O)NCc2ccnc3ccccc23)ccc1Oc1ccccc1C#N] | 178-180 | |
| 36 | 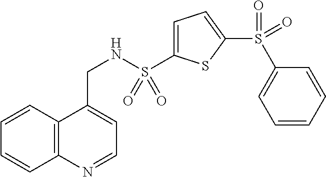 [SMILES: O=S(=O)(NCc1ccnc2ccccc12)c1ccc(S(=O)(=O)c2ccccc2)s1] | 111-112 | |
| 37 | 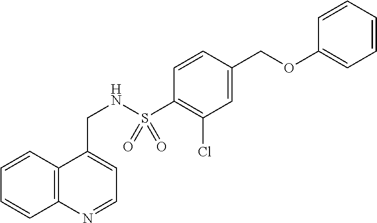 [SMILES: O=S(=O)(NCc1ccnc2ccccc12)c1ccc(COc2ccccc2)cc1Cl] | 126-128 | |
| 40 | 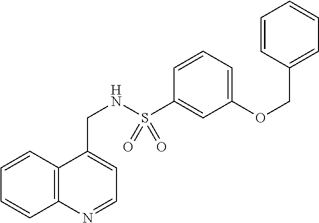 [SMILES: O=S(=O)(NCc1ccnc2ccccc12)c1cccc(OCc2ccccc2)c1] | 103-104 | |
| 41 | 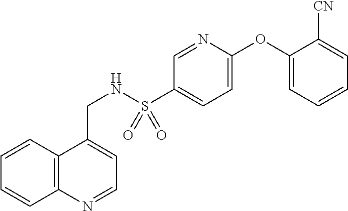 [SMILES: N#Cc1ccccc1Oc1ccc(S(=O)(=O)NCc2ccnc3ccccc23)cn1] | 118-121 | |
| 42 | 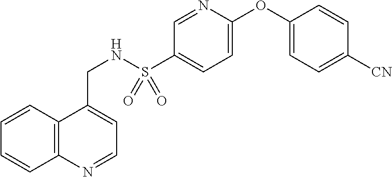 [SMILES: N#Cc1ccc(Oc2ccc(S(=O)(=O)NCc3ccnc4ccccc34)cn2)cc1] | 165-166 | |
| 43 | 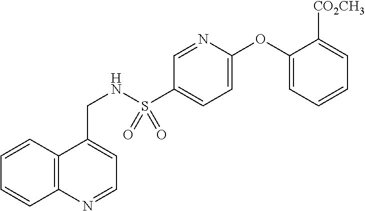 [SMILES: COC(=O)c1ccccc1Oc1ccc(S(=O)(=O)NCc2ccnc3ccccc23)cn1] | 167-169 | |
| 44 | 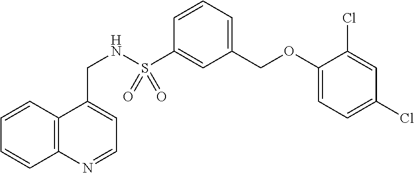 [SMILES: O=S(=O)(NCc1ccnc2ccccc12)c1cccc(COc2ccc(Cl)cc2Cl)c1] | 155-156 | |
| 45 | 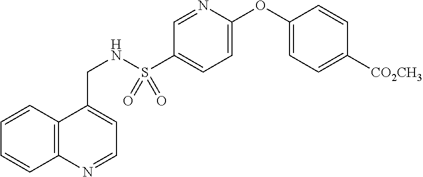 [SMILES: COC(=O)c1ccc(Oc2ccc(S(=O)(=O)NCc3ccnc4ccccc34)cn2)cc1] | 115-117 | |
| 46 | 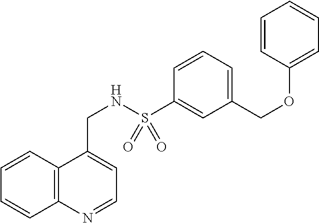 [SMILES: O=S(=O)(NCc1ccnc2ccccc12)c1cccc(COc2ccccc2)c1] | 108-109 | |
| 50 | 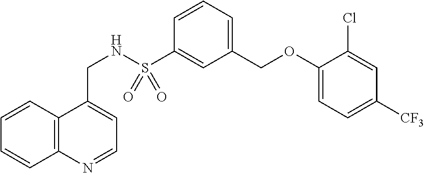 [SMILES: O=S(=O)(NCc1ccnc2ccccc12)c1cccc(COc2ccc(C(F)(F)F)cc2Cl)c1] | 150-151 | |
| 64 | 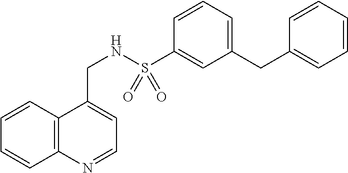 [SMILES: O=S(=O)(NCc1ccnc2ccccc12)c1cccc(Cc2ccccc2)c1] | | 389 |
| 72 | 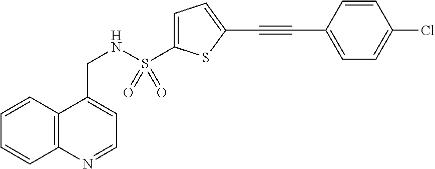 [SMILES: O=S(=O)(NCc1ccnc2ccccc12)c1ccc(C#Cc2ccc(Cl)cc2)s1] | 181-184 | |
| 73 | 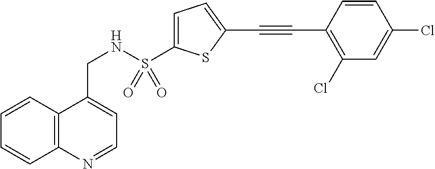 [SMILES: O=S(=O)(NCc1ccnc2ccccc12)c1ccc(C#Cc2ccc(Cl)cc2Cl)s1] | 161-164 | |
| 74 | 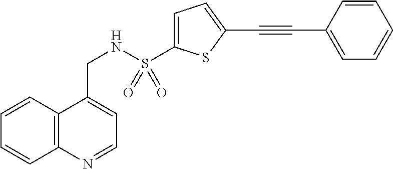 [SMILES: O=S(=O)(NCc1ccnc2ccccc12)c1ccc(C#Cc2ccccc2)s1] | 86-89 | |
| 75 | 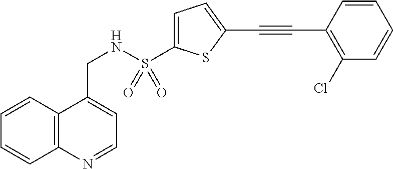 [SMILES: O=S(=O)(NCc1ccnc2ccccc12)c1ccc(C#Cc2ccccc2Cl)s1] | 143-146 | |
| 80 | 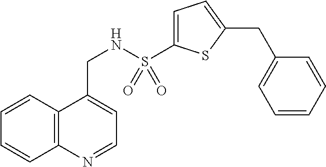 [SMILES: O=S(=O)(NCc1ccnc2ccccc12)c1ccc(Cc2ccccc2)s1] | 134-136 | |
| 82 | 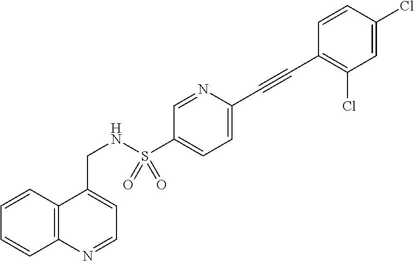 [SMILES: O=S(=O)(NCc1ccnc2ccccc12)c1ccc(C#Cc2ccc(Cl)cc2Cl)nc1] | 188-190 | |
| 86 | 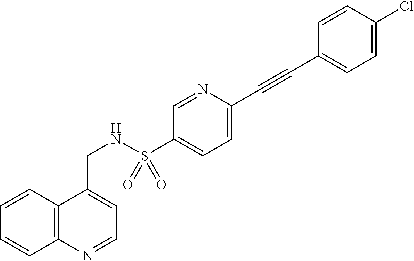 [SMILES: O=S(=O)(NCc1ccnc2ccccc12)c1ccc(C#Cc2ccc(Cl)cc2)nc1] | 179-181 | |
| 88 | 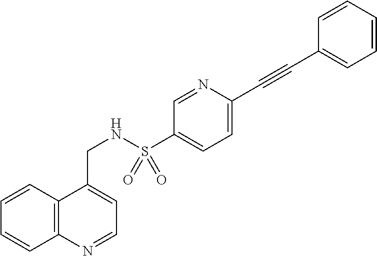 [SMILES: O=S(=O)(NCc1ccnc2ccccc12)c1ccc(C#Cc2ccccc2)nc1] | 163-165 | |
| 92 | 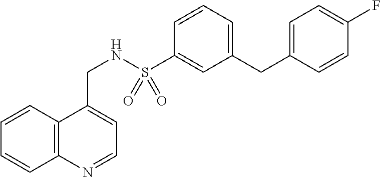 [SMILES: O=S(=O)(NCc1ccnc2ccccc12)c1cccc(Cc2ccc(F)cc2)c1] | | 407 |
| 99 | 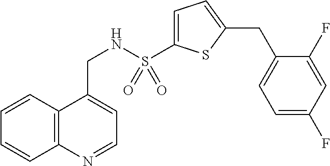 [SMILES: O=S(=O)(NCc1ccnc2ccccc12)c1ccc(Cc2ccc(F)cc2F)s1] | | 431 |
| 100 | 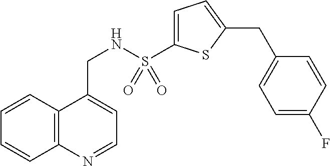 [SMILES: O=S(=O)(NCc1ccnc2ccccc12)c1ccc(Cc2ccc(F)cc2)s1] | | 413 |
| 101 | 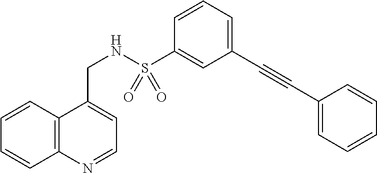 [SMILES: O=S(=O)(NCc1ccnc2ccccc12)c1cccc(C#Cc2ccccc2)c1] | 146-148 | |
| 108 | 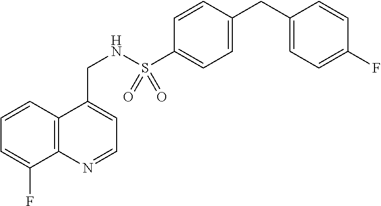 [SMILES: O=S(=O)(NCc1ccnc2c(F)cccc12)c1ccc(Cc2ccc(F)cc2)cc1] | | 425 |
| 111 | 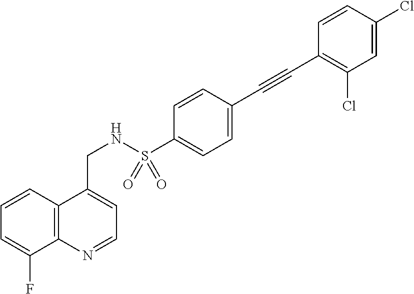 [SMILES: O=S(=O)(NCc1ccnc2c(F)cccc12)c1ccc(C#Cc2ccc(Cl)cc2Cl)cc1] | 204-206 | |
| 112 | 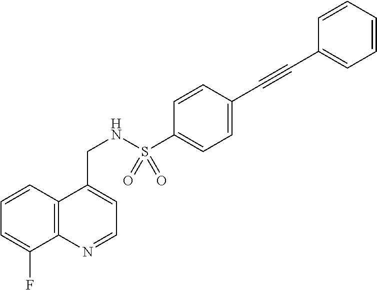 [SMILES: O=S(=O)(NCc1ccnc2c(F)cccc12)c1ccc(C#Cc2ccccc2)cc1] | 190-192 | |

INDEX TABLE C

| Cmpd No. | ¹H NMR Data^{a} |
|---|---|
| 24 | δ (DMSO-d₆) 2.92 (m, 4H), 4.53 (d, 2H), 7.25 (d, 2H), |
| | 7.31 (d, 2H), 7.42 (t, 3H), 7.60 (t, 1H), 7.75 (m, 3H), |
| | 8.01 (d, 1H), 8.12 (d, 1H), 8.31 (m, 1H), 8.82 (d, 1H). |
| 34 | δ (DMSO-d₆) 4.51 (d, 2H), 5.22 (s, 2H), 7.05 (d, 2H), |
| | 7.37 (d, 2H), 7.44 (d, 1H), 7.61 (m, 3H), 7.74 (t, 1H), |
| | 7.86 (d, 2H), 8.01 (d, 2H), 8.39 (t, 1H), 8.82 (d, 1H). |

^{a}¹H NMR data are in ppm downfield from tetramethylsilane. CDCl₃ solution unless indicated otherwise. DMSO-d₆ is CD₃S(O)CD₃. Couplings are designated by (s)—singlet, (d)—doublet, (t)—triplet, (m)—multiplet, (dd)—doublet of doublets, (br s)—broad singlet.

The following Tests demonstrate the control efficacy of compounds of this invention on specific parasitic pests. The pest control protection afforded by the compounds is not limited, however, to these species. Compound numbers refer to compounds in Index Tables A-B.

### BIOLOGICAL EXAMPLES OF THE INVENTION

### Test A

For evaluating control of the barber pole worm (Haemonchus contortus), a test compound was solubilized in culture media (Earle's Balanced Salt Solution) containing Haemonchus contortus eggs to obtain a final test compound concentration of 2.0 ppm. The test unit was evaluated for mortality 120 hours later after which the eggs had hatched and had advanced to the L3 stage.

Of the compounds tested, the following caused 100% mortality: 1, 2, 4, 5, 6, 7, 8, 9, 10, 11, 12, 13, 14, 15, 16, 17, 18, 19, 20, 21, 22, 23, 24, 25, 26, 27, 28, 31, 32, 34, 35, 36, 37, 38, 39, 40, 41, 44, 46, 47, 51, 52, 53, 55, 56, 58, 59, 60, 64, 65, 69, 70, 71, 73, 74, 75, 76, 77, 78, 79, 80, 83, 84, 85, 87, 89, 90, 91, 92, 94, 95, 96, 97, 98 and 99.

### Test B

For evaluating control of the barber pole worm (Haemonchus contortus), mice were each infected orally with 600 L3 Hlaemonchus contortus larvae on Day −3. On day 0, the infected mice were gavaged with a test compound (n=1) in a propylene glycol/glycerol formal solution at the rate of 10.0 mg/kg body weight. On day 5, the mice were euthanized and evaluated for Haemonchus contortus burdens relative to the vehicle-dosed controls. The range of means for the number of Haemonchus contortus in the various tests in which these compounds were studied was 55-184.

| Compound Number | % Efficacy |
|---|---|
| 2 | 76 |
| 5 | 69 |
| 7 | 61 |
| 9 | 72 |
| 15 | 79 |
| 23 | 54 |
| 27 | 78 |
| 28 | 62 |
| 31 | 66 |
| 34 | 71 |
| 38 | 75 |
| 51 | 74 |
| 53 | 61 |
| 56 | 78 |
| 59 | 86 |
| 60 | 76 |
| 64 | 70 |
| 69 | 90 |
| 70 | 89 |
| 74 | 60 |
| 76 | 87 |
| 80 | 80 |
| 91 | 93 |
| 92 | 73 |

### Test C

For evaluating control of the barber pole worm (Haemonchus contortus), lambs weighing approximately 35 Kg were each orally infected with 10,000 Haemonchus contortus L3 larvae on day −36. Fecal egg counts were done on day −1 to determine worm burdens. On day 0, the infected lambs were gavaged with a test compound (n=1) in a propylene glycoliglycerol formal solution at the rate of 5.0 mg/kg body weight. On day 8, the lambs were euthanized and evaluated for Haemonchus contortus burdens relative to the vehicle-dosed controls. Of the compounds tested, the following caused ≧75% reduction of adult worms: 5 and 59.

What is claimed is:

1. A compound of Formula 1, an N-oxide, or salt thereof, 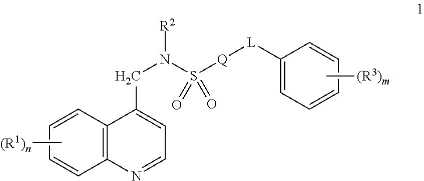
wherein
Q is phenyl or naphthalenyl each optionally substituted with up to 5 substituents independently selected from R^{4a}; or
Q is a 5- to 6-membered heteroaromatic ring or an 8- to 11-membered heteroaromatic bicyclic ring system, each ring or ring system containing ring members selected from carbon atoms and up to 4 heteroatoms independently selected from up to 2 O, up to 2 S and up to 4 N atoms, and optionally substituted with up to 5 substituents independently selected from R^{4a} on carbon atom ring members and R^{4b} on nitrogen atom ring members;
L is (CR^{13a}R^{13b})ₜ, CR¹⁴═CR¹⁴, C≡C, CR^{15a}R^{15b}X, XCR^{15a}R^{15b}, CO, O, S(O)ₚ, NR⁵, CONR⁵, NR⁵CO, NR⁵SO₂ or SO₂NR⁵;
X is O, S, SO, SO₂, or NR⁵;
each R¹ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
R² is hydrogen, cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₁-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
each R³ is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl or C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
each R^{4a} is independently halogen, cyano, nitro, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl, or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
R^{4b} is cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
R⁵ is hydrogen, cyano, OR⁶, NR^{7a}R^{7b}, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, OR⁶, NR^{7a}R^{7b}, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, OR⁶ and S(O)ₚR¹²;
each R⁶ is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, C₁-C₆ alkoxy and S(O)ₚR¹²;
each R^{7a} is independently hydrogen, C(O)R⁸, C(O)OR⁹, C(O)NR¹⁰R¹¹, S(O)ₚR¹² or S(O)₂NR¹⁰R¹¹; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹; or C₃-C₇ cycloalkyl, C₄-C₈ cycloalkylalkyl or C₅-C₇ cycloalkenyl, each optionally substituted with substituents independently selected from the group consisting of halogen, cyano, nitro, C₁-C₄ alkyl, C₁-C₄ haloalkyl, C₁-C₆ alkoxy and S(O)ₚR¹²;
each R^{7b} is independently hydrogen; or C₁-C₆ alkyl, C₂-C₆ alkenyl, C₂-C₆ alkynyl or benzyl each optionally substituted with substituents independently selected from the group consisting of halogen, cyano, nitro, C₁-C₆ alkoxy, C₁-C₆ alkylamino, C₂-C₈ dialkylamino, C(O)R⁸, C(O)OR⁹, C(O)NR¹⁰R¹¹, S(O)ₚR¹² and S(O)₂NR¹⁰R¹¹;
R⁸, R⁹, R¹⁰ and R¹² are each independently hydrogen; or C₁-C₆ alkyl, C₂-C₆ alkenyl, C₂-C₆ alkynyl, phenyl, benzyl, C₃-C₇ cycloalkyl, C₄-C₈ cycloalkylalkyl or C₅-C₇ cycloalkenyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₂-C₆ alkoxycarbonyl, C₂-C₆ alkylaminocarbonyl, C₂-C₈ dialkylaminocarbonyl, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl and C₁-C₄ haloalkylsulfonyl;
each R¹¹ is independently hydrogen; or C₁-C₆ alkyl, C₁-C₆ alkenyl, C₂-C₆ alkynyl or benzyl each optionally substituted with substituents independently selected from the group consisting of halogen, cyano, nitro, C₁-C₄ alkyl, C₁-C₄ haloalkyl, C₁-C₄ alkoxy, C₁-C₄ haloalkoxy, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl and C₁-C₄ haloalkylsulfonyl;
each R^{13a} is independently hydrogen, halogen, cyano, hydroxyl or amino; or C₂-C₆ alkoxycarbonyl, C₂-C₆ alkylaminocarbonyl or C₂-C₈ dialkylaminocarbonyl; or C₁-C₆ alkyl, C₁-C₆ alkenyl, C₂-C₆ alkynyl, C₃-C₇ cycloalkyl, C₄-C₈ cycloalkylalkyl, phenyl or benzyl each optionally substituted with substituents independently selected from the group consisting of halogen, cyano, nitro, C₁-C₄ alkyl, C₁-C₄ alkoxy, C₁-C₄ haloalkyl, C₁-C₄ haloalkoxy, C₂-C₆ alkoxycarbonyl, C₁-C₆ alkylaminocarbonyl and C₂-C₈ dialkylaminocarbonyl;
each R^{13b} is independently hydrogen, halogen, or C₁-C₆ alkyl;
each R¹⁴ is independently hydrogen, halogen, cyano, C₁-C₆ alkyl or C₁-C₆ haloalkyl;
R^{15a} is hydrogen, cyano, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₇ cycloalkyl, C₄-C₈ cycloalkylalkyl, C₅-C₇ cycloalkenyl, phenyl, benzyl, C₂-C₆ alkoxycarbonyl, C₂-C₆ alkylaminocarbonyl or C₂-C₈ dialkylaminocarbonyl;
R^{15b} is hydrogen or C₁-C₆ alkyl;
n is 0, 1, 2, 3, 4 or 5;
m is 0, 1, 2, 3, 4 or 5;
p is 0, 1 or 2; and
t is 1, 2 or 3;
provided that when
L is an oxygen atom, then one R³ group is other than hydrogen, halogen, C₁-C₄ alkyl, C₁-C₄ alkoxy, C₁-haloalkyl or C₁-haloalkoxy.

2. A compound of claim 1 wherein
Q is a ring selected from the group consisting of 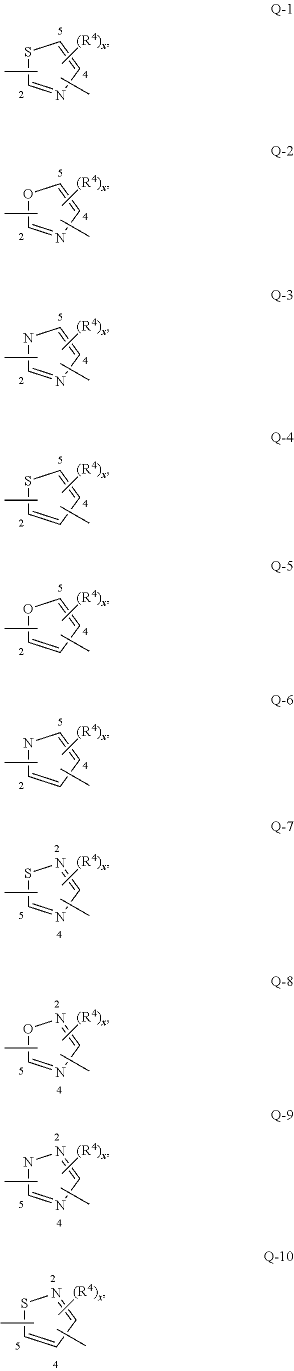 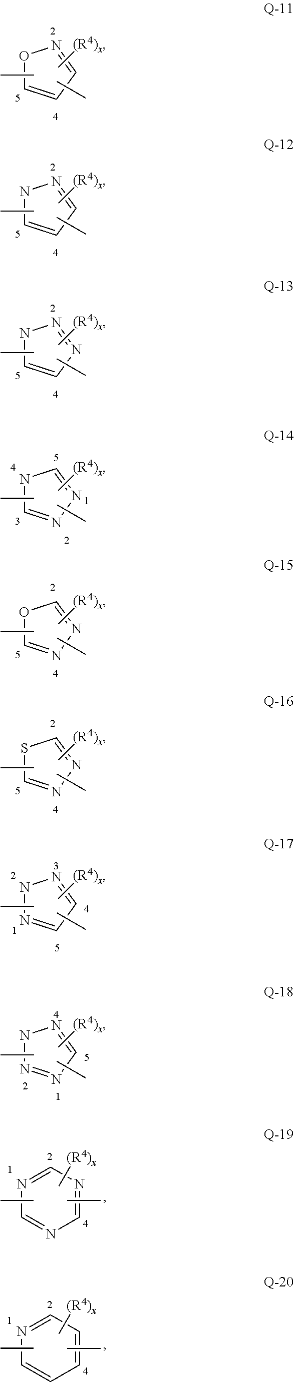 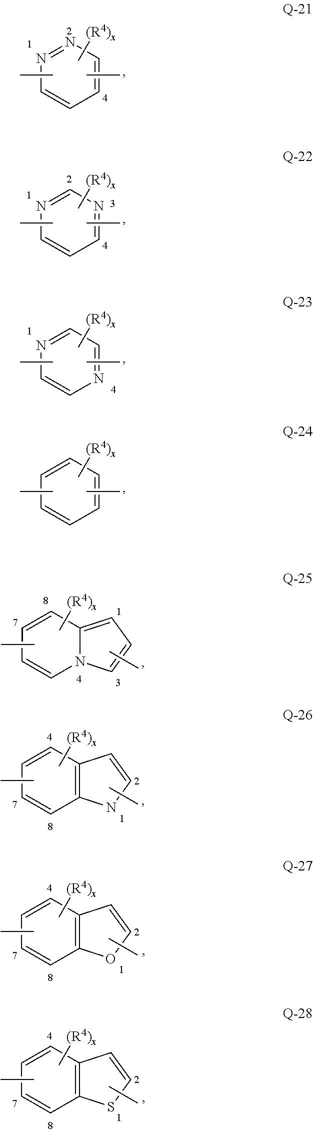 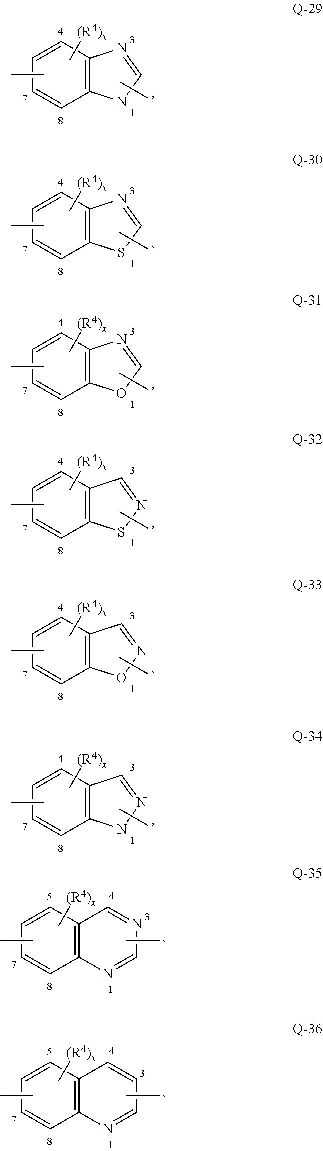 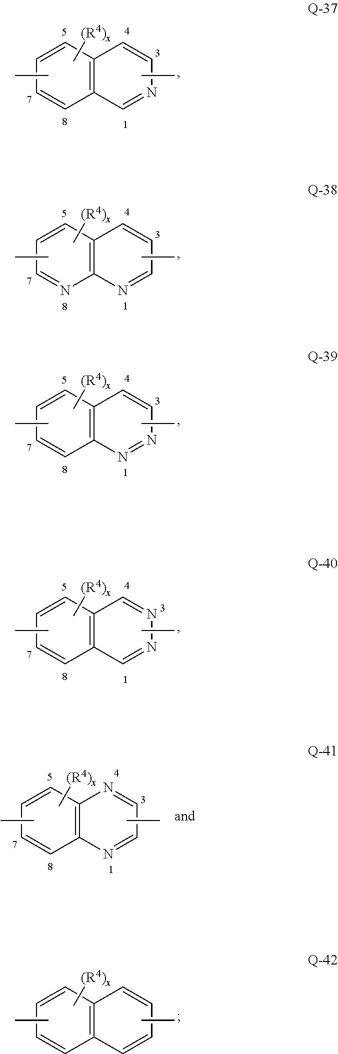
wherein one of the floating bonds is connected to SO₂ in Formula 1 through any available carbon or nitrogen atom of the depicted ring or ring system and the other floating bond is connected to L in Formula 1 through any available carbon or nitrogen atom of the depicted ring or ring system; when R⁴ is attached to a carbon ring member, said R⁴ is selected from R^{4a}, and when R⁴ is attached to a nitrogen ring member, said R⁴ is selected from R^{4b}; and x is an integer from 0 to 5;
L is (CR^{13a}R^{13b})ₜ, C≡C or CR^{15a}R^{15b}X;
X is O;
each R¹ is independently halogen, cyano, nitro, OR⁶, C₁-C₃ alkyl or C₁-C₃ haloalkyl;
each R^{4a} is independently halogen, cyano, nitro, OR⁶, C₁-C₆ alkyl or C₁-C₆ haloalkyl;
R^{4b} is methyl; and
n is 0, 1 or 2.

3. A compound of claim 2 wherein
Q is Q-24;
x is 0, 1, 2 or 3;
L is (CR^{13a}R^{13b})ₜ;
t is 1;
each R^{13a} is independently hydrogen, halogen or C₁-C₂ alkyl;
R² is hydrogen or methyl;
each R³ is independently halogen, cyano, nitro, OR⁶, NR^{7a}R^{7b}, C(O)R⁸, C(O)OR⁹, C(O)NR¹⁰R¹¹, S(O)ₚR¹², S(O)₂NR¹⁰R¹¹, C₁-C₆ alkyl or C₁-C₆ haloalkyl;
each R⁶ is independently hydrogen, C₁-C₆ alkyl or C₁-C₆ haloalkyl; and
m is 0, 1 or 2.

4. A compound of claim 3 wherein
each R¹ is independently fluorine, chlorine, CH₃, CF₃, OCF₃ or OCHF₂;
R² is hydrogen; and
each R³ is independently halogen, cyano, OR⁶, S(O)ₚR¹², C₁-C₄ alkyl or C₁-C₄ haloalkyl.

5. A compound of claim 1 wherein
Q is Q-24;
x is 0, 1, 2 or 3;
L is O;
each R¹ is independently halogen, cyano, nitro, OR⁶, C₁-C₃ alkyl or C₁-C₃ haloalkyl;
R² is hydrogen or methyl;
each R³ is independently cyano, nitro, OR⁶, NR^{7a}R^{7b}, C(O)R^{S}, C(O)OR⁹, C(O)NR¹⁰R¹¹, S(O)ₚR¹², S(O)₂NR¹⁰R¹¹, C₅-C₆ alkyl or C₂-C₆ haloalkyl;
each R^{4a} is independently halogen, cyano, nitro, OR⁶, C₁-C₆ alkyl or C₁-C₆ haloalkyl;
each R⁶ is independently hydrogen, C₅-C₆ alkyl and C₁-C₆ haloalkyl;
n is 0, 1 or 2; and
m is 0, 1 or 2.

6. A compound of claim 1 that is selected from the group consisting of:
4-(3-chloro-2-cyanophenoxy)-N-(4-quinolinylmethyl)benzenesulfonamide;
4-(phenylmethyl)-N-(4-quinolinylmethyl)benzenesulfonamide;
4-(4-cyanophenoxy)-N-(4-quinolinylmethyl)benzenesulfonamide;
4-(2-chloro-4-cyanophenoxy)-N-(4-quinolinylmethyl)benzenesulfonamide;
4-(2-cyanophenoxy)-N-(4-quinolinylmethyl)benzenesulfonamide;
4-(phenoxymethyl)-N-(4-quinolinylmethyl)benzenesulfonamide;
4-[2-(4-chlorophenyl)ethynyl]-N-(4-quinolinylmethyl)benzenesulfonamide;
4-(3-cyanophenoxy)-N-(4-quinolinylmethyl)benzenesulfonamide;
4-[2-(2,4-dichlorophenyl)ethynyl]-N-(4-quinolinylmethyl-benzenesulfonamide; and
4-[(4-chlorophenoxy)methyl]-N-(4-quinolinylmethyl)benzenesulfonamide.

7. A compound of claim 1 that is:
4-[(4-fluorophenyl)methyl]-N-(4-quinolinylmethyl)benzenesulfonamide.

8. A composition comprising a parasiticidally effective amount of a compound of claim 1, and at least one pharmaceutically or veterinarily acceptable carrier or diluent

9. A composition comprising (a) a parasiticidally effective amount of a compound of claim 1; and (b) a at least one additional biologically active compound or agent.

10. A method for treating, controlling, preventing or protecting animals against infection by helminths which comprises orally, topically, parenterally or subcutaneously administering to the animals a parasiticdally effective amount of a compound of claim 1, or a pharmaceutically or veterinarily acceptable salt or a composition comprising it.

11. The method of claim 10 wherein the administration is enteral.

12. The method of claim 11 wherein the administration is oral.

13. The method of claim 10 wherein the administration is parenteral.

14. The method of claim 10 wherein the application is topical.

15. The method of claim 10 wherein the helminth is Haemonchus contortus.

16. The method of claim 15 wherein the administration is oral.
